(19) United States
(12) Patent Application Publication
(10) Pub. No.: US 2025/0059154 A1
(43) Pub. Date: Feb. 20, 2025

(54) CYSTEINE-DEPENDENT INVERSE AGONISTS OF NUCLEAR RECEPTORS ROR-GAMMA/ROR-GAMMA-T AND METHODS OF TREATING DISEASES OR DISORDERS THEREWITH

(71) Applicant: REATA PHARMACEUTICALS, INC., Irving, TX (US)

(72) Inventors: Melean VISNICK, Irving, TX (US); Xin JIANG, Coppell, TX (US)

(73) Assignee: REATA PHARMACEUTICALS, INC., Irving, TX (US)

(21) Appl. No.: 18/770,647

(22) Filed: Jul. 11, 2024

Related U.S. Application Data
(62) Division of application No. 17/252,841, filed on Dec. 16, 2020, now Pat. No. 12,060,340.
(60) Provisional application No. 62/687,782, filed on Jun. 20, 2018.

(51) Int. Cl. C07D 401/04; C07D 403/04

(52) U.S. Cl. CPC C07D 401/04; C07D 403/04

(57) ABSTRACT

Provided herein are compounds, compositions and methods for treating and preventing diseases and disorders, comprising administering to patients therapeutically effective amounts of cysteine-dependent inverse agonists of the nuclear receptor RORγ/RORγt. In some such embodiments, the inverse agonists bind to cysteine 476 of a nuclear receptor RORγ in the patient. Also provided are methods, as well as compounds and compositions, for modulating the activity of RORγ and RORγt by binding to the allosteric binding site through a covalent bond. In some aspects, the present disclosure relates to using compounds containing Michael acceptor groups which bind to a cysteine residue in the allosteric binding site such as cysteine 476 in RORγ.

This application is a divisional of U.S. application Ser. No. 17/252,841, filed Dec. 16, 2020, as a national phase application under 35 U.S.C. § 371 of International Application No. PCT/US2019/038337, filed Jun. 20, 2019, which claims the benefit of U.S. Provisional Application No. 62/687,782, filed on Jun. 20, 2018, the entire contents of each of which are hereby incorporated by reference.

### REFERENCE TO A SEQUENCE LISTING

This application contains a Sequence Listing XML, which has been submitted electronically and is hereby incorporated by reference in its entirety. Said XML Sequence Listing, created on Jul. 11, 2024, is named REATP0106USD1.xml and is 3,829 bytes in size.

### BACKGROUND

### I. Field

The present invention relates generally to the fields of biology and medicine. More particularly, it concerns compounds, compositions, and methods for the treatment and prevention of diseases such as those associated with RAR-related orphan receptor γ (RORγ) or RORγt and excess production of IL-17.

### II. Description of Related Art

Since the discovery of synthetic ligands for the progesterone receptor by Djerassi and colleagues at Syntex in 1950, the field of nuclear receptor (NR) ligand dependent transcription factors has proven fertile territory for drug discovery and development. Not surprisingly, when the lymphocyte specific NR RORγt was shown by Littman to be both necessary and sufficient for IL-17 expression and T helper 17 (Th17) cell differentiation in human CD4+ T cells, a flurry of drug discovery activity ensued. The Th17 cell subset produces several pro-inflammatory cytokines (e.g., IL-17A, IL-17F, GM-CSF and IL-22), and has been shown to be the major pathogenic population in animal models of autoimmune inflammation, such as the experimental autoimmune encephalomyelitis (EAE) model for multiple sclerosis and the collagen-induce arthritis (CIA) model for rheumatoid arthritis. With the FDA approval of several biological agents targeting the IL-17/IL-23 pathway clinical proof of concept for the roles of these cytokines in human autoimmune diseases has been unambiguously established. Vitae Pharmaceuticals was first to enter the clinic with an orally active RORγt inverse agonist, VTP43742, that showed significant efficacy in a Phase 2a psoriasis clinical study. VTP43742 and the majority of orally active agents that modulate RORγt target the classical orthosteric binding pocket in the ligand binding domain (LBD) of RORγt. Recently, Merck Research Laboratories and Eindhoven University have described a series of molecules that antagonize RORγt function through binding to an allosteric site on the LBD through non-covalent interactions. Therefore, methods of modulating the activity of RORγ, especially RORγt, by covalent modulation of the allosteric site in the ligand binding region are of particular interest.

### SUMMARY

The present disclosure provides compounds, methods and compositions that may be used, for example, to modulating the activity of RORγ and RORγt by binding to a cysteine residue at an allosteric site.

In one aspect, there are provided methods for treating diseases and disorders in patients in need thereof, comprising administering to the patient a therapeutically effective amount of a cysteine-dependent inverse agonist of the nuclear receptor RORγ/RORγt.

In another aspect, the present disclosure provides pharmaceutical compositions comprising a cysteine-dependent inverse agonist of nuclear receptor RORγ/RORγt in an amount sufficient to modulate the activity of nuclear receptor RORγ/RORγt when administered to a patient.

Either or both of the above methods and pharmaceutical compositions may be combined with any of the following embodiments. In some embodiments, the therapeutically effective amount is sufficient to modulate the activity of the patient's nuclear receptor RORγ/RORγt. In some embodiments, the inverse agonist binds to cysteine 476 of a nuclear receptor RORγ in the patient. In some embodiments, the binding occurs via the formation of a covalent bond between the inverse agonist and the cysteine 476. In some embodiments, the inverse agonist binds selectively to cysteine 476 of a nuclear receptor RORγ in the patient. In some embodiments, the inverse agonist binds preferentially to cysteine 476 of a nuclear receptor RORγ relative to the orthosteric binding pocket (i.e., the cholesterol binding site) in the ligand binding domain (LBD) of RORγ in the patient. In some embodiments, the inverse agonist does not bind to any significant extent to the orthosteric binding pocket in the ligand binding domain (LBD) of RORγ in the patient. In some embodiments, the inverse agonist inhibits the patient's RORγ/RORγt activity without significantly affecting the patient's RORα or RORβ activity.

In some embodiments, the method or pharmaceutical composition results in modulating the function of the patient's RORγ/RORγt. In some embodiments, the method or pharmaceutical composition results in suppressing the patient's interleukin-17A production. In some embodiments, the method or pharmaceutical composition results in selectively inhibiting the patient's T helper 17 (Th17) cell differentiation. In some embodiments, the inverse agonist's RORγt-LBD-GAL4 reporter assay IC₅₀ activity is less than 1 M. In further embodiments, the inverse agonist's RORγt-LBD-GAL4 reporter assay IC₅₀ activity is less than 500 nM. In still further embodiments, the inverse agonist's RORγt-LBD-GAL4 reporter assay IC₅₀ activity is less than 100 nM. In some embodiments, the inverse agonist's RORγt-LBD-GAL4 reporter assay IC₅₀ activity is less than 1,000 nM, 950 nM, 900 nM, 850 nM, 800 nM, 750 nM, 700 nM, 650 nM, 600 nM, 550 nM, 500 nM, 450 nM, 400 nM, 350 nM, 300 nM, 250 nM, 200 nM, 150 nM, 100 nM, 75 nM, 50 nM, 25 nM, 20 nM, 15 nM or 10 nM. In some embodiments, the inverse agonist's suppression of IL-17A secretion from human CD4+ T-cells assay IC₅₀ activity is less than 500 nM. In further embodiments, the inverse agonist's suppression of IL-17A secretion from human CD4+ T-cells assay IC₅₀ activity is less than 100 nM. In still further embodiments, the inverse agonist's suppression of IL-17A secretion from human CD4+ T-cells assay IC₅₀ activity is less than 50 nM. In some embodiments, the inverse agonist's suppression of IL-17A secretion from human CD4+ T-cells assay IC₅₀ activity is less than 500 nM, 450 nM, 400 nM, 350 nM, 300 nM, 250 nM, 200 nM, 150 nM, 100 nM, 75 nM, 50 nM, 25 nM, 20 nM, 15 nM or 10 nM. In some embodiments, the disease or disorder is an autoimmune disease. In further embodiments, the autoimmune disease is Crohn's disease, rheumatoid arthritis, lupus, or psoriasis. In still further embodiments, the autoimmune disease is rheumatoid arthritis or psoriasis.

In some embodiments, the chemical formula of the inverse agonist comprises a chemical group of the formula:

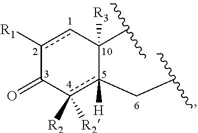

wherein:
the bond between carbon atoms 1 and 2 is an epoxidized double bond or a double bond;
the bond between carbon atoms 4 and 5 is a single bond or a double bond;
R₁ is cyano, heteroaryl_{(C≤8)}, substituted heteroaryl_{(C≤8)}, —CF₃, or —C(O)Rₐ; wherein:
   Rₐ is hydroxy, amino, or alkoxy_{(C≤8)}, alkylamino_{(C≤8)}, dialkylamino_{(C≤8)}, alkylsulfonylamino_{(C≤8)}, or a substituted version of any of these groups;
R₂ is hydrogen or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of any of these groups, or -alkanediyl_{(C≤8)}-cycloalkyl_{(C≤12)} or a substituted version of this group;
R₂′ is absent, hydrogen, or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of these groups; provided that when the bond between carbon atoms 4 and 5 is a double bond then R₂′ is absent; and
R₃ is alkyl_{(C≤12)}, alkenyl_{(C≤12)}, aryl_{(C≤12)}, aralkyl_{(C≤12)}, or a substituted version of any of these groups.

In some embodiments, the formula is further defined as:

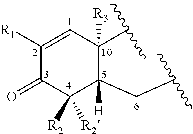

wherein:
R¹ is cyano, heteroaryl_{(C≤8)}, substituted heteroaryl_{(C≤8)}, —CF₃, or —C(O)Rₐ; wherein:
   Rₐ is hydroxy, amino, or alkoxy_{(C≤8)}, alkylamino_{(C≤8)}, dialkylamino_{(C≤8)}, alkylsulfonylamino_{(C≤8)}, or a substituted version of any of these groups;
R₂ is hydrogen or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of any of these groups, or -alkanediyl_{(C≤8)}-cycloalkyl_{(C≤12)} or a substituted version of this group;
R₂′ is absent, hydrogen, or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of these groups; provided that when the bond between carbon atoms 4 and 5 is a double bond then R₂′ is absent; and
R₃ is alkyl_{(C≤12)}, alkenyl_{(C≤12)}, aryl_{(C≤12)}, aralkyl_{(C≤12)}, or a substituted version of any of these groups.

In some embodiments, the formula is further defined as:

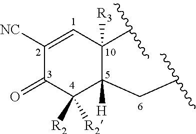

wherein:
R₂ is hydrogen or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of any of these groups, or -alkanediyl_{(C≤8)}-cycloalkyl_{(C≤12)} or a substituted version of this group;
R₂′ is absent, hydrogen, or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of these groups; provided that when the bond between carbon atoms 4 and 5 is a double bond then R₂′ is absent; and
R₃ is alkyl_{(C≤12)}, alkenyl_{(C≤12)}, aryl_{(C≤12)}, aralkyl_{(C≤12)}, or a substituted version of any of these groups.

In some embodiments, the formula is further defined as:

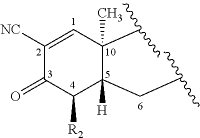

wherein:
R₂ is hydrogen or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of any of these groups, or -alkanediyl_{(C≤8)}-cycloalkyl_{(C≤12)} or a substituted version of this group.

In some embodiments, the formula is further defined as:

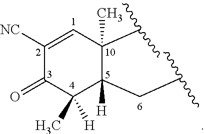 [SMILES: *C[C@@H]1[C@@H](C)C(=O)C(C#N)=C[C@]1(*)C]

In some embodiments, the inverse agonist is a compound of the formula:

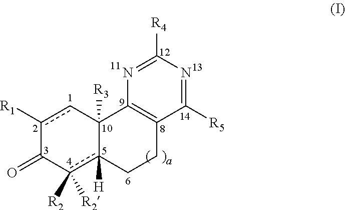

wherein:
the bond between carbon atoms 1 and 2 is an epoxidized double bond or a double bond;
the bond between carbon atoms 4 and 5 is a single bond or a double bond;
a is 0, 1, or 2;
R₁ is cyano, heteroaryl_{(C≤8)}, substituted heteroaryl_{(C≤8)}, —CF₃, or —C(O)Rₐ; wherein:
   Rₐ is hydroxy, amino, or alkoxy_{(C≤8)}, alkylamino_{(C≤8)}, dialkylamino_{(C≤8)}, alkylsulfonylamino_{(C≤8)}, or a substituted version of any of these groups;
R₂ is hydrogen or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of any of these groups, or -alkanediyl_{(C≤8)}-cycloalkyl_{(C≤12)} or a substituted version of this group;
R₂′ is absent, hydrogen, or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of these groups; provided that when the bond between carbon atoms 4 and 5 is a double bond then R₂′ is absent;
R₃ is alkyl_{(C≤12)}, alkenyl_{(C≤12)}, aryl_{(C≤12)}, aralkyl_{(C≤12)}, or a substituted version of any of these groups;
R₄ is hydrogen, amino, alkyl_{(C≤8)}, substituted alkyl_{(C≤8)}, cycloalkyl_{(C≤8)}, substituted cycloalkyl_{(C≤18)}, aryl_{(C≤18)}, substituted aryl_{(C≤18)}, aralkyl_{(C≤18)}, substituted aralkyl_{(C≤18)}, heteroaryl_{(C≤18)}, substituted heteroaryl_{(C≤18)}, heteroaralkyl_{(C≤18)}, substituted heteroaralkyl_{(C≤18)}, heterocycloalkyl_{(C≤8)}, substituted heterocycloalkyl_{(C≤18)}, amido_{(C≤18)}, substituted amido_{(C≤18)}, or

—X₁—(CH₂)ₘ—R₄′;
wherein:
   X₁ is NR_{b}, O, or S; wherein:
      R_{b} is hydrogen, alkyl_{(C≤6)}, or substituted alkyl_{(C≤6)};
   m is 0, 1, 2, 3, or 4; and
   R₄′ is alkyl_{(C≤12)}, cycloalkyl_{(C≤12)}, aryl_{(C≤18)}, aralkyl_{(C≤18)}, heteroaryl_{(C≤18)}, heteroaralkyl_{(C≤8)}, heterocycloalkyl_{(C≤18)}, or a substituted version of any of these groups; or

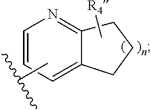

wherein:
   n is 0, 1, 2, 3, or 4; and
   R⁴″ is —H, —OH, —F, —Cl, —Br, —I, —NH₂, —NO₂, —CN, —SH, —S(O)₂OH, or —S(O)₂NH₂, or alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; or

—X₂—(CH₂)ₚ—R₄′″
wherein:
   X₂ is arenediyl_{(C≤12)}, substituted arenediyl_{(C≤12)}, heterocycloalkanediyl_{(C≤12)}, substituted heterocycloalkanediyl_{(C≤12)}, heteroarenediyl_{(C≤12)}, or substituted heteroarenediyl_{(C≤12)};
   p is 0, 1, 2, 3, or 4; and
   R₄′″ is alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; and
R₅ is amino, hydroxy, —OS(O)₂C₆H₄CH₃, alkyl_{(C≤12)}, alkoxy_{(C≤12)}, cycloalkyl_{(C≤12)}, cycloalkoxy_{(C≤12)}, aryl_{(C≤12)}, aralkyl_{(C≤12)}, heteroaryl_{(C≤12)}, heteroaralkyl_{(C≤12)}, heterocycloalkyl_{(C≤12)}, acyl_{(C≤12)}, acyloxy_{(C≤12)}, alkylamino_{(C≤12)}, dialkylamino_{(C≤12)}, alkylsulfonylamino_{(C≤12)}, or a substituted version of any of the last fourteen groups, or

—OY₁-A₁;
wherein:
   Y₁ is alkanediyl_{(C≤8)} or substituted alkanediyl_{(C≤8)}; and
   A₁ is cycloalkyl_{(C≤8)} or substituted cycloalkyl_{(C≤8)}; or

—Y₂—C(O)NR_{c}-A₂;
wherein:
   Y₂ is arenediyl_{(C≤8)} or substituted arenediyl_{(C≤8)};
   R_{c} is hydrogen, alkyl_{(C≤6)}, or substituted alkyl_{(C≤6)}; and
   A₂ is aralkyl_{(C≤12)} or substituted aralkyl_{(C≤12)}; or

-A₃R_{d};
wherein:
   A₃ is —O— or —NRₑ—, wherein
      Rₑ is hydrogen, alkyl_{(C≤6)}, or substituted alkyl_{(C≤6)}; and
R_{d} is acyl_{(C≤12)}, or substituted acyl_{(C≤12)}; provided that when carbon atoms 4 and 5 are joined by a double bond, then R₂′ and the
hydrogen atom at carbon atom 5 are absent;
   or a pharmaceutically acceptable salt thereof.

In some embodiments, the compound is further defined as:

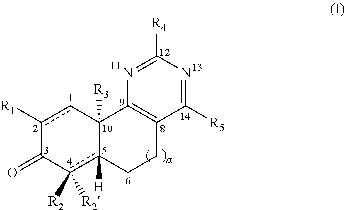

wherein:
the bond between carbon atoms 1 and 2 is an epoxidized double bond or a double bond;
the bond between carbon atoms 4 and 5 is a single bond or a double bond;
a is 0, 1, or 2;
R¹ is cyano, heteroaryl_{(C≤8)}, substituted heteroaryl_{(C≤8)}, —CF₃, or —C(O)Rₐ; wherein:
   Rₐ is hydroxy, amino, or alkoxy_{(C≤8)}, alkylamino_{(C≤8)}, dialkylamino_{(C≤8)}, alkylsulfonylamino_{(C≤8)}, or a substituted version of any of these groups;
R₂ is hydrogen or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of any of these groups, or -alkanediyl_{(C≤8)}-cycloalkyl_{(C≤12)} or a substituted version of this group;
R₂′ is absent, hydrogen, or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of these groups; provided that when the bond between carbon atoms 4 and 5 is a double bond then R₂′ is absent;
R₃ is alkyl_{(C≤12)}, alkenyl_{(C≤12)}, aryl_{(C≤12)}, aralkyl_{(C≤12)}, or a substituted version of any of these groups;
R₄ is hydrogen, amino, alkyl_{(C2-8)}, substituted alkyl_{(C≤18)}, cycloalkyl_{(C≤18)}, substituted cycloalkyl_{(C≤18)}, aryl_{(C≤18)}, substituted aryl_{(C≤18)}, aralkyl_{(C≤18)}, substituted aralkyl_{(C≤18)}, heteroaryl_{(C≤18)}, substituted heteroaryl_{(C≤18)}, heteroaralkyl_{(C≤18)}, substituted heteroaralkyl_{(C≤18)}, heterocycloalkyl_{(C≤18)}, substituted heterocycloalkyl_{(C≤18)}, amido_{(C≤18)}, substituted amido_{(C≤18)}, or

—X₁—(CH₂)ₘ—R₄′;
wherein:
   X₁ is NR_{b}, O, or S; wherein:
      R_{b} is hydrogen, alkyl_{(C≤6)}, or substituted alkyl_{(C≤6)}; m is 0, 1, 2, 3, or 4; and
   R₄′ is alkyl_{(C≤12)}, cycloalkyl_{(C≤8)}, aryl_{(C≤18)}, aralkyl_{(C≤18)}, heteroaryl_{(C≤18)}, heteroaralkyl_{(C≤8)}, heterocycloalkyl_{(C≤8)}, or a substituted version of any of these groups, provided that when X₁ is 0, then R₄′ is not methyl; or

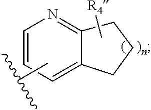

wherein:
   n is 0, 1, 2, 3, or 4; and
   R⁴″ is —H, —OH, —F, —Cl, —Br, —I, —NH₂, —NO₂, —CN, —SH, —S(O)₂OH, or —S(O)₂NH₂, or alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; or

—X₂—(CH₂)ₚ—R₄′″;
wherein:
   X₂ is arenediyl_{(C≤12)}, substituted arenediyl_{(C≤12)}, heterocycloalkanediyl_{(C≤12)}, substituted heterocycloalkanediyl_{(C≤12)}, heteroarenediyl_{(C≤12)}, or substituted heteroarenediyl_{(C≤12)};
   p is 0, 1, 2, 3, or 4; and
   R₄′ is alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; and
R₅ is amino, hydroxy, —OS(O)₂C₆H₄CH₃, alkyl_{(C≤12)}, alkoxy_{(C≤12)}, cycloalkyl_{(C≤12)}, cycloalkoxy_{(C≤12)}, aryl_{(C≤12)}, aralkyl_{(C≤12)}, heteroaryl_{(C≤12)}, heteroaralkyl_{(C≤12)}, heterocycloalkyl_{(C≤12)}, acyl_{(C≤12)}, acyloxy_{(C≤12)}, alkylamino_{(C≤12)}, dialkylamino_{(C≤12)}, alkylsulfonylamino_{(C≤12)}, or a substituted version of any of the last fourteen groups, or

—OY₁-A₁;
wherein:
   Y₁ is alkanediyl_{(C≤8)} or substituted alkanediyl_{(C≤8)}; and
   A₁ is cycloalkyl_{(C≤8)} or substituted cycloalkyl_{(C≤8)}; or

—Y₂—C(O)NRₑ-A₂;
wherein:
   Y₂ is arenediyl_{(C≤8)} or substituted arenediyl_{(C≤8)};
   R_{c} is hydrogen, alkyl_{(C≤6)}, or substituted alkyl_{(C≤6)}; and
   A₂ is aralkyl_{(C≤12)} or substituted aralkyl_{(C≤12)}; or

-A₃R_{d};
wherein:
   A₃ is —O— or —NRₑ—, wherein
      Rₑ is hydrogen, alkyl_{(C≤6)}, or substituted alkyl_{(C≤6)}; and
   R_{d} is acyl_{(C≤12)}, or substituted acyl_{(C≤12)};
provided that when carbon atoms 4 and 5 are joined by a double bond, then R₂′ and the hydrogen atom at carbon atom 5 are absent;
   or a pharmaceutically acceptable salt thereof.

In some embodiments, the compound is further defined as:

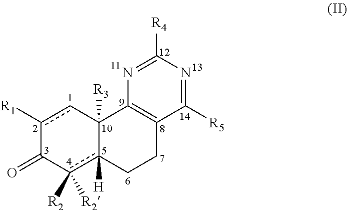

wherein:
the bond between carbon atoms 1 and 2 is an epoxidized double bond or a double bond;
the bond between carbon atoms 4 and 5 is a single bond or a double bond;
R₁ is cyano, heteroaryl_{(C≤8)}, substituted heteroaryl_{(C≤8)}, —CF₃, or —C(O)Rₐ; wherein:
   Rₐ is hydroxy, amino, or alkoxy_{(C≤8)}, alkylamino_{(C≤8)}, dialkylamino_{(C≤8)}, alkylsulfonylamino_{(C≤8)}, or a substituted version of any of these groups;
R₂ is hydrogen or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of any of these groups, or -alkanediyl_{(C≤8)}-cycloalkyl_{(C≤12)} or a substituted version of this group;
R₂′ is absent, hydrogen, alkyl_{(C≤12)}, cycloalkyl_{(C≤12)}, alkenyl_{(C≤12)}, alkynyl_{(C≤12)}, or a substituted version of the last four groups; provided that when the bond between carbon atoms 4 and 5 is a double bond then R₂′ is absent;
R₃ is alkyl_{(C≤12)}, alkenyl_{(C≤12)}, aryl_{(C≤12)}, aralkyl_{(C≤12)}, or a substituted version of any of these groups;
R₄ is hydrogen, amino, alkyl_{(C2-8)}, substituted alkyl_{(C≤18)}, cycloalkyl_{(C≤18)}, substituted cycloalkyl_{(C≤8)}, aryl_{(C≤18)}, substituted aryl_{(C≤18)}, aralkyl_{(C≤18)}, substituted aralkyl_{(C≤18)}, heteroaryl_{(C≤18)}, substituted heteroaryl_{(C≤18)}, heteroaralkyl_{(C≤18)}, substituted heteroaralkyl_{(C≤18)}, heterocycloalkyl_{(C≤8)}, substituted heterocycloalkyl_{(C≤18)}, alkylamino_{(C≤18)}, substituted alkylamino_{(C≤18)}, dialkylamino_{(C≤18)}, substituted dialkylamino_{(C≤18)}, alkylthio_{(C≤18)}, substituted alkylthio_{(C≤18)}, amido_{(C≤18)}, substituted amido_{(C≤8)}, or

—X₁—(CH₂)ₘ—R₄′;
wherein:
   X₁ is NR_{b}, O, or S; wherein:
      R_{b} is hydrogen, alkyl_{(C≤6)}, or substituted alkyl_{(C≤6)};
   m is 0, 1, 2, 3, or 4; and
   R₄′ is alkyl_{(C≤12)}, cycloalkyl_{(C≤18)}, aryl_{(C≤18)}, aralkyl_{(C≤18)}, heteroaryl_{(C≤18)}, heteroaralkyl_{(C≤8)}, heterocycloalkyl_{(C≤18)}, or a substituted version of any of these groups, provided that when X₁ is O, then R₄′ is not methyl; or

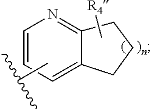

wherein:
   n is 0, 1, 2, 3, or 4; and
   R₄″ is —H, —OH, —F, —Cl, —Br, —I, —NH₂, —NO₂, —CN, —SH, —S(O)₂OH, or —S(O)₂NH₂, or alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; or

—X₂—(CH₂)ₚ—R₄′″;
wherein:
   X₂ is arenediyl_{(C≤12)}, substituted arenediyl_{(C≤12)}, heterocycloalkanediyl_{(C≤12)}, substituted heterocycloalkanediyl_{(C≤12)}, heteroarenediyl_{(C≤12)}, or substituted heteroarenediyl_{(C≤12)};
   p is 0, 1, 2, 3, or 4; and
   R₄′″ is alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; and
R₅ is amino, hydroxy, —OS(O)₂C₆H₄CH₃, alkyl_{(C≤12)}, alkoxy_{(C≤12)}, cycloalkyl_{(C≤12)}, cycloalkoxy_{(C≤12)}, aryl_{(C≤12)}, aralkyl_{(C≤12)}, heteroaryl_{(C≤12)}, heteroaralkyl_{(C≤12)}, heterocycloalkyl_{(C≤12)}, acyl_{(C≤12)}, acyloxy_{(C≤12)}, alkylamino_{(C≤12)}, dialkylamino_{(C≤12)}, alkylsulfonylamino_{(C≤12)}, or a substituted version of any of the last fourteen groups, or

—OY₁-A₁;
wherein:
   Y₁ is alkanediyl_{(C≤8)} or substituted alkanediyl_{(C≤8)}; and
   A₁ is cycloalkyl_{(C≤8)} or substituted cycloalkyl_{(C≤8)}; or

—Y₂—C(O)NRₑ-A₂;
wherein:
   Y₂ is arenediyl_{(C≤8)} or substituted arenediyl_{(C≤8)};
   R_{c} is hydrogen, alkyl_{(C≤6)}, or substituted alkyl_{(C≤6)}; and
   A₂ is aralkyl_{(C≤12)} or substituted aralkyl_{(C≤12)}; or

-A₃R_{d};
wherein:
   A₃ is —O— or —NRₑ—, wherein
      Rₑ is hydrogen, alkyl_{(C≤6)}, or substituted alkyl_{(C≤6)}; and
   R_{d} is acyl_{(C≤12)}, or substituted acyl_{(C≤12)};
provided that when carbon atoms 4 and 5 are joined by a double bond, then R₂′ and the hydrogen atom at carbon atom 5 are absent;
   or a pharmaceutically acceptable salt thereof.

In some embodiments, the compound is further defined as:

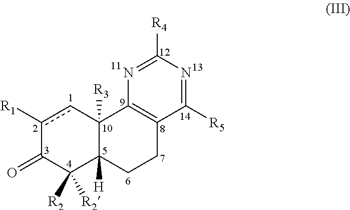

wherein:
the bond between carbon atoms 1 and 2 is an epoxidized double bond or a double bond;
R¹ is cyano, heteroaryl_{(C≤8)}, substituted heteroaryl_{(C≤8)}, —CF₃, or —C(O)Rₐ; wherein:
   Rₐ is hydroxy, amino, or alkoxy_{(C≤8)}, alkylamino_{(C≤8)}, dialkylamino_{(C≤8)}, alkylsulfonylamino_{(C≤8)}, or a substituted version of any of these groups;
R₂ is hydrogen or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of any of these groups, or -alkanediyl_{(C≤8)}-cycloalkyl_{(C≤12)} or a substituted version of this group;
R₂′ is hydrogen, alkyl_{(C≤12)}, cycloalkyl_{(C≤12)}, alkenyl_{(C≤12)}, alkynyl_{(C≤12)}, or a substituted version of the last four groups;
R₃ is alkyl_{(C≤12)}, aryl_{(C≤12)}, aralkyl_{(C≤12)}, or a substituted version of any of these groups;
R₄ is hydrogen, amino, alkyl_{(C2-8)}, substituted alkyl_{(C≤8)}, cycloalkyl_{(C≤8)}, substituted cycloalkyl_{(C≤8)}, aryl_{(C≤8)}, substituted aryl_{(C≤8)}, aralkyl_{(C≤8)}, substituted aralkyl_{(C≤8)}, heteroaryl_{(C≤8)}, substituted heteroaryl_{(C≤8)}, heteroaralkyl_{(C≤18)}, substituted heteroaralkyl_{(C≤8)}, heterocycloalkyl_{(C≤8)}, substituted heterocycloalkyl_{(C≤18)}, alkylamino_{(C≤18)}, substituted alkylamino_{(C≤18)}, dialkylamino_{(C≤8)}, substituted dialkylamino_{(C≤18)}, alkylthio_{(C≤18)}, substituted alkylthio_{(C≤18)}, amido_{(C≤18)}, substituted amido_{(C≤18)}, or

—X₁—(CH₂)ₘ—R₄′;
wherein:
   X₁ is NR_{b}, O, or S; wherein:
      R_{b} is hydrogen, alkyl_{(C≤6)}, or substituted alkyl_{(C≤6)};
   m is 0, 1, 2, 3, or 4; and
   R₄′ is alkyl_{(C≤12)}, cycloalkyl_{(C≤18)}, aryl_{(C≤18)}, aralkyl_{(C≤18)}, heteroaryl_{(C≤18)}, heteroaralkyl_{(C≤18)}, heterocycloalkyl_{(C≤8)}, or a substituted version of any of these groups, provided that when X₁ is O, then R₄′ is not methyl; or

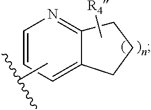

wherein:
   n is 0, 1, 2, 3, or 4; and
   R₄″ is —H, —OH, —F, —Cl, —Br, —I, —NH₂, —NO₂, —CN, —SH, —S(O)₂OH, or —S(O)₂NH₂, or alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; or

—X₂—(CH₂)ₚ—R₄′″;
wherein:
   X₂ is arenediyl_{(C≤12)}, substituted arenediyl_{(C≤12)}, heterocycloalkanediyl_{(C≤12)}, substituted heterocycloalkanediyl_{(C≤12)}, heteroarenediyl_{(C≤12)}, or substituted heteroarenediyl_{(C≤12)};
   p is 0, 1, 2, 3, or 4; and
   R₄′″ is alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; and
R₅ is amino, hydroxy, —OS(O)₂C₆H₄CH₃, alkyl_{(C≤12)}, alkoxy_{(C≤12)}, cycloalkyl_{(C≤12)}, cycloalkoxy_{(C≤12)}, aryl_{(C≤12)}, aralkyl_{(C≤12)}, heteroaryl_{(C≤12)}, heterocycloalkyl_{(C≤12)}, acyl_{(C≤12)}, acyloxy_{(C≤12)}, alkylamino_{(C≤12)}, dialkylamino_{(C≤12)}, alkylsulfonylamino_{(C≤12)}, or a substituted version of any of the last fourteen groups, or

—OY₁-A₁;
wherein:
   Y₁ is alkanediyl_{(C≤8)} or substituted alkanediyl_{(C≤8)}; and
   A₁ is cycloalkyl_{(C≤8)} or substituted cycloalkyl_{(C≤8)}; or

—Y₂—C(O)NR_{b}-A₂;
wherein:
   Y₂ is arenediyl_{(C≤8)} or substituted arenediyl_{(C≤8)};
   R_{b} is hydrogen, alkyl_{(C≤6)}, or substituted alkyl_{(C≤6)}; and
   A₂ is aralkyl_{(C≤12)} or substituted aralkyl_{(C≤12)}; or

-A₃R_{d};
wherein:
   A₃ is —O— or —NRₑ—, wherein
      Rₑ is hydrogen, alkyl_{(C≤6)}, or substituted alkyl_{(C≤6)}; and
   R_{d} is acyl_{(C≤12)}, or substituted acyl_{(C≤12)};
      or a pharmaceutically acceptable salt thereof.

In some embodiments, the compound is further defined as:

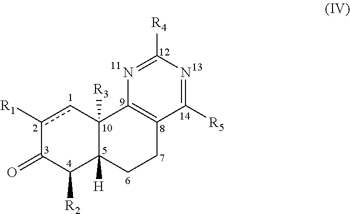

wherein:
the bond between carbon atoms 1 and 2 is an epoxidized double bond or a double bond;
R₁ is cyano, heteroaryl_{(C≤8)}, substituted heteroaryl_{(C≤8)}, —CF₃, or —C(O)Rₐ; wherein:
   Rₐ is hydroxy, amino, or alkoxy_{(C≤8)}, alkylamino_{(C≤8)}, dialkylamino_{(C≤8)}, alkylsulfonylamino_{(C≤8)}, or a substituted version of any of these groups;
R₂ is hydrogen or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of any of these groups, or -alkanediyl_{(C≤8)}-cycloalkyl_{(C≤12)} or a substituted version of this group;
R₃ is alkyl_{(C≤12)}, aryl_{(C≤12)}, aralkyl_{(C≤12)}, or a substituted version of any of these groups;
R₄ is hydrogen, amino, alkyl_{(C2-18)}, substituted alkyl_{(C≤18)}, cycloalkyl_{(C≤18)}, substituted cycloalkyl_{(C≤18)}, aryl_{(C≤18)}, substituted aryl_{(C≤18)}, aralkyl_{(C≤18)}, substituted aralkyl_{(C≤18)}, heteroaryl_{(C≤18)}, substituted heteroaryl_{(C≤18)}, heteroaralkyl_{(C≤18)}, substituted heteroaralkyl_{(C≤18)}, heterocycloalkyl_{(C≤18)}, substituted heterocycloalkyl_{(C≤18)}, alkylamino_{(C≤18)}, substituted alkylamino_{(C≤18)}, dialkylamino_{(C≤18)}, substituted dialkylamino_{(C≤18)}, alkylthio_{(C≤18)}, substituted alkylthio_{(C≤18)}, amido_{(C≤18)}, substituted amido_{(C≤18)}, or

—X₁—(CH₂)ₘ—R₄′;
wherein:
   X₁ is NR_{b}, O, or S; wherein:
      R_{b} is hydrogen, alkyl_{(C≤6)}, or substituted alkyl_{(C≤6)};
   m is 0, 1, 2, 3, or 4; and
   R₄′ is alkyl_{(C≤12)}, cycloalkyl_{(C≤18)}, aryl_{(C≤18)}, aralkyl_{(C≤18)}, heteroaryl_{(C≤18)}, heteroaralkyl_{(C≤18)}, heterocycloalkyl_{(C≤18)}, or a substituted version of any of these groups, provided that when X₁ is O, then R₄′ is not methyl; or

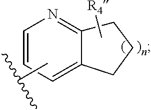

wherein:
   n is 0, 1, 2, 3, or 4; and
   R⁴″ is —H, —OH, —F, —Cl, —Br, —I, —NH₂, —NO₂, —CN, —SH, —S(O)₂OH, or —S(O)₂NH₂, or alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; or

—X₂—(CH₂)ₚ—R₄′″;
wherein:
   X₂ is arenediyl_{(C≤12)}, substituted arenediyl_{(C≤12)}, heterocycloalkanediyl_{(C≤12)}, substituted heterocycloalkanediyl_{(C≤12)}, heteroarenediyl_{(C≤12)}, or substituted heteroarenediyl_{(C≤12)};
   p is 0, 1, 2, 3, or 4; and
   R₄′″ is alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; and
R₅ is amino, hydroxy, —OS(O)₂C₆H₄CH₃, alkyl_{(C≤12)}, alkoxy_{(C≤12)}, cycloalkyl_{(C≤12)}, cycloalkoxy_{(C≤12)}, aryl_{(C≤12)}, aralkyl_{(C≤12)}, heteroaryl_{(C≤12)}, heterocycloalkyl_{(C≤12)}, acyl_{(C≤12)}, acyloxy_{(C≤12)}, alkylamino_{(C≤12)}, dialkylamino_{(C≤12)}, alkylsulfonylamino_{(C≤12)}, or a substituted version of any of the last fourteen groups, or

—OY₁-A₁;
wherein:
   Y₁ is alkanediyl_{(C≤8)} or substituted alkanediyl_{(C≤8)}; and
   A₁ is cycloalkyl_{(C≤8)} or substituted cycloalkyl_{(C≤8)}; or

—Y₂—C(O)NR_{b}-A₂;
wherein:
   Y₂ is arenediyl_{(C≤8)} or substituted arenediyl_{(C≤8)};
   R_{b} is hydrogen, alkyl_{(C≤6)}, or substituted alkyl_{(C≤6)}; and
   A₂ is aralkyl_{(C≤12)} or substituted aralkyl_{(C≤12)}; or

-A₃R_{d};
wherein:
   A₃ is —O— or —NRₑ—, wherein
      Rₑ is hydrogen, alkyl_{(C≤6)}, or substituted alkyl_{(C≤6)}; and
   R_{d} is acyl_{(C≤12)}, or substituted acyl_{(C≤12)};
      or a pharmaceutically acceptable salt thereof.

In some embodiments, the compound is further defined as:

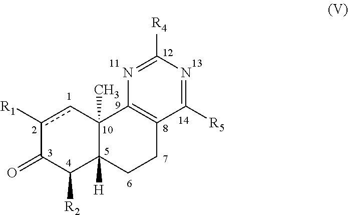

wherein:
the bond between carbon atoms 1 and 2 is an epoxidized double bond or a double bond;
R¹ is cyano, heteroaryl_{(C≤8)}, substituted heteroaryl_{(C≤8)}, —CF₃, or —C(O)Rₐ; wherein:
   Rₐ is hydroxy, amino, or alkoxy_{(C≤8)}, alkylamino_{(C≤8)}, dialkylamino_{(C≤8)}, alkylsulfonylamino_{(C≤8)}, or a substituted version of any of these groups;
R₂ is hydrogen or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of any of these groups, or -alkanediyl_{(C≤8)}-cycloalkyl_{(C≤12)} or a substituted version of this group;
R₄ is hydrogen, amino, alkyl_{(C2-8)}, substituted alkyl_{(C≤18)}, cycloalkyl_{(C≤18)}, substituted cycloalkyl_{(C≤18)}, aryl_{(C≤18)}, substituted aryl_{(C≤18)}, aralkyl_{(C≤18)}, substituted aralkyl_{(C≤18)}, heteroaryl_{(C≤18)}, substituted heteroaryl_{(C≤18)}, heteroaralkyl_{(C≤18)}, substituted heteroaralkyl_{(C≤18)}, heterocycloalkyl_{(C≤18)}, substituted heterocycloalkyl_{(C≤18)}, alkylamino_{(C≤18)}, substituted alkylamino_{(C≤18)}, dialkylamino_{(C≤18)}, substituted dialkylamino_{(C≤18)}, alkylthio_{(C≤18)}, substituted alkylthio_{(C≤18)}, amido_{(C≤18)}, substituted amido_{(C≤18)}, or

—X₁—(CH₂)ₘ—R₄′;
wherein:
   X₁ is NR_{b}, O, or S; wherein:
      R_{b} is hydrogen, alkyl_{(C≤6)}, or substituted alkyl_{(C≤6)};
   m is 0, 1, 2, 3, or 4; and
   R₄′ is alkyl_{(C≤12)}, cycloalkyl_{(C≤18)}, aryl_{(C≤18)}, aralkyl_{(C≤18)}, heteroaryl_{(C≤18)}, heteroaralkyl_{(C≤18)}, heterocycloalkyl_{(C≤18)}, or a substituted version of any of these groups, provided that when X₁ is O, then R₄′ is not methyl; or

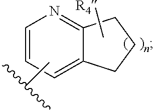

wherein:
   n is 0, 1, 2, 3, or 4; and
   R₄″ is —H, —OH, —F, —Cl, —Br, —I, —NH₂, —NO₂, —CN, —SH, —S(O)₂OH, or —S(O)₂NH₂, or alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; or

—X₂—(CH₂)ₚ—R₄′″;
wherein:
   X₂ is arenediyl_{(C≤12)}, substituted arenediyl_{(C≤12)}, heterocycloalkanediyl_{(C≤12)}, substituted heterocycloalkanediyl_{(C≤12)}, heteroarenediyl_{(C≤12)}, or substituted heteroarenediyl_{(C≤12)};
   p is 0, 1, 2, 3, or 4; and
   R₄′″ is alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; and
R₅ is amino, hydroxy, —OS(O)₂C₆H₄CH₃, alkyl_{(C≤12)}, alkoxy_{(C≤12)}, cycloalkyl_{(C≤12)}, cycloalkoxy_{(C≤12)}, aryl_{(C≤12)}, aralkyl_{(C≤12)}, heteroaryl_{(C≤12)}, heterocycloalkyl_{(C≤12)}, acyl_{(C≤12)}, acyloxy_{(C≤12)}, alkylamino_{(C≤12)}, dialkylamino_{(C≤12)}, alkylsulfonylamino_{(C≤12)}, or a substituted version of any of the last fourteen groups, or

—OY₁-A₁;
wherein:
   Y₁ is alkanediyl_{(C≤8)} or substituted alkanediyl_{(C≤8)}; and
   A₁ is cycloalkyl_{(C≤8)} or substituted cycloalkyl_{(C≤8)}; or

—Y₂—C(O)NR_{b}-A₂;
wherein:
   Y₂ is arenediyl_{(C≤8)} or substituted arenediyl_{(C≤8)};
   R_{b} is hydrogen, alkyl_{(C≤6)}, or substituted alkyl_{(C≤6)}; and
   A₂ is aralkyl_{(C≤12)} or substituted aralkyl_{(C≤12)}; or

-A₃R_{d};
wherein:
   A₃ is —O— or —NRₑ—, wherein
      Rₑ is hydrogen, alkyl_{(C≤6)}, or substituted alkyl_{(C≤6)}; and
   R_{d} is acyl_{(C≤12)}, or substituted acyl_{(C≤12)};
      or a pharmaceutically acceptable salt thereof.

In some embodiments, the compound is further defined as:

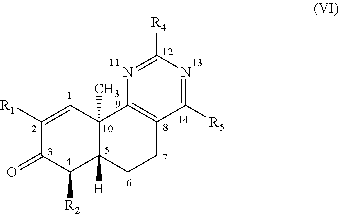

wherein:
R¹ is cyano, heteroaryl_{(C≤8)}, substituted heteroaryl_{(C≤8)}, —CF₃, or —C(O)Rₐ; wherein:
   Rₐ is hydroxy, amino, or alkoxy_{(C≤8)}, alkylamino_{(C≤8)}, dialkylamino_{(C≤8)}, alkylsulfonylamino_{(C≤8)}, or a substituted version of any of these groups;
R₂ is hydrogen or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of any of these groups, or -alkanediyl_{(C≤8)}-cycloalkyl_{(C≤12)} or a substituted version of this group;
R₄ is hydrogen, amino, alkyl_{(C2-8)}, substituted alkyl_{(C≤8)}, cycloalkyl_{(C≤8)}, substituted cycloalkyl_{(C≤8)}, aryl_{(C≤8)}, substituted aryl_{(C≤8)}, aralkyl_{(C≤8)}, substituted aralkyl_{(C≤8)}, heteroaryl_{(C≤8)}, substituted heteroaryl_{(C≤8)}, heteroaralkyl_{(C≤18)}, substituted heteroaralkyl_{(C≤18)}, heterocycloalkyl_{(C≤8)}, substituted heterocycloalkyl_{(C≤18)}, alkylamino_{(C≤18)}, substituted alkylamino_{(C≤18)}, dialkylamino_{(C≤18)}, substituted dialkylamino_{(C≤18)}, alkylthio_{(C≤18)}, substituted alkylthio_{(C≤8)}, amido_{(C≤8)}, substituted amido_{(C≤18)}, or

—X₁—(CH₂)ₘ—R₄′;
wherein:
   X₁ is NR_{b}, O, or S; wherein:
      R_{b} is hydrogen, alkyl_{(C≤6)}, or substituted alkyl_{(C≤6)}; m is 0, 1, 2, 3, or 4; and
   R₄′ is alkyl_{(C≤12)}, cycloalkyl_{(C≤8)}, aryl_{(C≤18)}, aralkyl_{(C≤18)}, heteroaryl_{(C≤18)}, heteroaralkyl_{(C≤18)}, heterocycloalkyl_{(C≤8)}, or a substituted version of any of these groups, provided that when X₁ is O, then R₄′ is not methyl; or

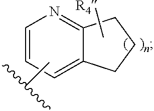

wherein:
   n is 0, 1, 2, 3, or 4; and
   R₄″ is —H, —OH, —F, —Cl, —Br, —I, —NH₂, —NO₂, —CN, —SH, —S(O)₂OH, or —S(O)₂NH₂, or alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; or

—X₂—(CH₂)ₚ—R₄′″;
wherein:
   X₂ is arenediyl_{(C≤12)}, substituted arenediyl_{(C≤12)}, heterocycloalkanediyl_{(C≤12)}, substituted heterocycloalkanediyl_{(C≤12)}, heteroarenediyl_{(C≤12)}, or substituted heteroarenediyl_{(C≤12)};
   p is 0, 1, 2, 3, or 4; and
   R₄′″ is alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; and
R₅ is amino, hydroxy, —OS(O)₂C₆H₄CH₃, alkyl_{(C≤12)}, alkoxy_{(C≤12)}, cycloalkyl_{(C≤12)}, cycloalkoxy_{(C≤12)}, aryl_{(C≤12)}, aralkyl_{(C≤12)}, heteroaryl_{(C≤12)}, heterocycloalkyl_{(C≤12)}, acyl_{(C≤12)}, acyloxy_{(C≤12)}, alkylamino_{(C≤12)}, dialkylamino_{(C≤12)}, alkylsulfonylamino_{(C≤12)}, or a substituted version of any of the last fourteen groups, or

—OY₁-A₁;
wherein:
   Y₁ is alkanediyl_{(C≤8)} or substituted alkanediyl_{(C≤8)}; and
   A₁ is cycloalkyl_{(C≤8)} or substituted cycloalkyl_{(C≤8)}; or

—Y₂—C(O)NR_{b}-A₂;
wherein:
   Y₂ is arenediyl_{(C≤8)} or substituted arenediyl_{(C≤8)};
   R_{b} is hydrogen, alkyl_{(C≤6)}, or substituted alkyl_{(C≤6)}; and
   A₂ is aralkyl_{(C≤12)} or substituted aralkyl_{(C≤12)}; or

-A₃R_{d};
wherein:
   A₃ is —O— or —NRₑ—, wherein
      Rₑ is hydrogen, alkyl_{(C≤6)}, or substituted alkyl_{(C≤6)}; and
   R_{d} is acyl_{(C≤12)}, or substituted acyl_{(C≤12)};
      or a pharmaceutically acceptable salt thereof.

In some embodiments, the compound is further defined as:

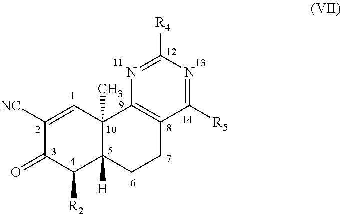

wherein:
R₂ is hydrogen or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, acyl_{(C≤12)}, or a substituted version of any of these groups, or -alkanediyl_{(C≤8)}-cycloalkyl_{(C≤12)} or a substituted version of this group;
R₄ is hydrogen, amino, alkyl_{(C2-8)}, substituted alkyl_{(C≤18)}, cycloalkyl_{(C≤18)}, substituted cycloalkyl_{(C≤8)}, aryl_{(C≤18)}, substituted aryl_{(C≤18)}, aralkyl_{(C≤18)}, substituted aralkyl_{(C≤18)}, heteroaryl_{(C≤18)}, substituted heteroaryl_{(C≤18)}, heteroaralkyl_{(C≤18)}, substituted heteroaralkyl_{(C≤18)}, heterocycloalkyl_{(C≤18)}, substituted heterocycloalkyl_{(C≤18)}, alkylamino_{(C≤18)}, substituted alkylamino_{(C≤18)}, dialkylamino_{(C≤18)}, substituted dialkylamino_{(C≤18)}, alkylthio_{(C≤18)}, substituted alkylthio_{(C≤18)}, amido_{(C≤8)}, substituted amido_{(C≤8)}, or

—X₁—(CH₂)ₘ—R₄′;
wherein:
   X₁ is NR_{b}, O, or S; wherein:
      R_{b} is hydrogen, alkyl_{(C≤6)}, or substituted alkyl_{(C≤6)};
   m is 0, 1, 2, 3, or 4; and
   R₄′ is alkyl_{(C≤12)}, cycloalkyl_{(C≤8)}, aryl_{(C≤18)}, aralkyl_{(C≤18)}, heteroaryl_{(C≤18)}, heteroaralkyl_{(C≤18)}, heterocycloalkyl_{(C≤8)}, or a substituted version of any of these groups, provided that when X₁ is O, then R₄′ is not methyl; or

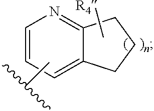

wherein:
   n is 0, 1, 2, 3, or 4; and
   R⁴″ is —H, —OH, —F, —Cl, —Br, —I, —NH₂, —NO₂, —CN, —SH, —S(O)₂OH, or —S(O)₂NH₂, or alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; or

—X₂—(CH₂)ₚ—R₄′″
wherein:
   X₂ is arenediyl_{(C≤12)}, substituted arenediyl_{(C≤12)}, heterocycloalkanediyl_{(C≤12)}, substituted heterocycloalkanediyl_{(C≤12)}, heteroarenediyl_{(C≤12)}, or substituted heteroarenediyl_{(C≤12)};
   p is 0, 1, 2, 3, or 4; and
   R₄′″ is alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; and
R₅ is amino, hydroxy, —OS(O)₂C₆H₄CH₃, alkyl_{(C≤12)}, alkoxy_{(C≤12)}, cycloalkyl_{(C≤12)}, cycloalkoxy_{(C≤12)}, aryl_{(C≤12)}, aralkyl_{(C≤12)}, heteroaryl_{(C≤12)}, heterocycloalkyl_{(C≤12)}, acyl_{(C≤12)}, acyloxy_{(C≤12)}, alkylamino_{(C≤12)}, dialkylamino_{(C≤12)}, alkylsulfonylamino_{(C≤12)}, or a substituted version of any of the last fourteen groups, or

—OY₁-A₁;
wherein:
   Y₁ is alkanediyl_{(C≤8)} or substituted alkanediyl_{(C≤8)}; and
   A₁ is cycloalkyl_{(C≤8)} or substituted cycloalkyl_{(C≤8)}; or

—Y₂—C(O)NR_{b}-A₂;
wherein:
   Y₂ is arenediyl_{(C≤8)} or substituted arenediyl_{(C≤8)};
   R_{b} is hydrogen, alkyl_{(C≤6)}, or substituted alkyl_{(C≤6)}; and
   A₂ is aralkyl_{(C≤12)} or substituted aralkyl_{(C≤12)}; or

-A₃R_{d};
wherein:
   A₃ is —O— or —NRₑ—, wherein
      Rₑ is hydrogen, alkyl_{(C≤6)}, or substituted alkyl_{(C≤6)}; and
   R_{d} is acyl_{(C≤12)}, or substituted acyl_{(C≤12)};
      or a pharmaceutically acceptable salt thereof.

In some embodiments, the compound is further defined as:

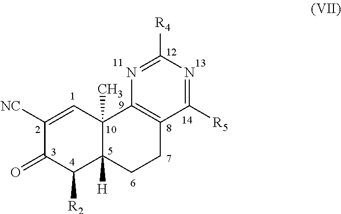

wherein:
R₂ is hydrogen, alkyl_{(C≤12)}, or substituted alkyl_{(C≤12)};
R₄ is heteroaryl_{(C≤18)} or substituted heteroaryl_{(C≤18)}; and
R₅ is aryl_{(C≤12)} or substituted aryl_{(C≤12)};
   or a pharmaceutically acceptable salt thereof.

In some embodiments, the compound is further defined as:

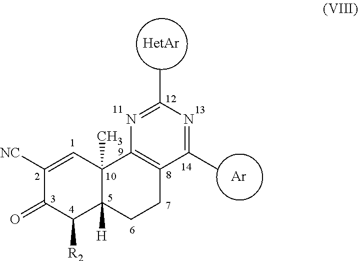

wherein:
R₂ is alkyl_{(C≤12)} or substituted alkyl_{(C≤12)};
HetAr is heteroaryl_{(C≤18)} or substituted heteroaryl_{(C≤18)}; and
Ar is aryl_{(C≤12)} or substituted aryl_{(C≤12)};
   or a pharmaceutically acceptable salt thereof.

In some embodiments, the compound is further defined as:

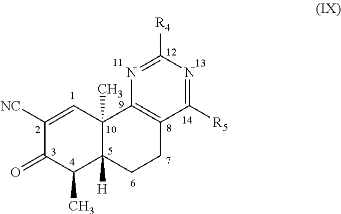

wherein:
R₄ is heteroaryl_{(C≤18)} or substituted heteroaryl_{(C≤18)}; and
R₅ is aryl_{(C≤12)} or substituted aryl_{(C≤12)};
   or a pharmaceutically acceptable salt thereof.

In some embodiments, the inverse agonist is a compound of the formula:

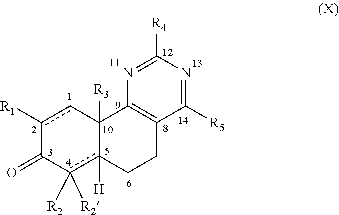

wherein:
the bond between carbon atoms 1 and 2 is an epoxidized double bond or a double bond;
the bond between carbon atoms 4 and 5 is a single bond or a double bond;
a is 0, 1, or 2;
R¹ is cyano, heteroaryl_{(C≤8)}, substituted heteroaryl_{(C≤8)}, —CF₃, or —C(O)Rₐ; wherein:
   Rₐ is hydroxy, amino, or alkoxy_{(C≤8)}, alkylamino_{(C≤8)}, dialkylamino_{(C≤8)}, alkylsulfonylamino_{(C≤8)}, or a substituted version of any of these groups;
R₂ is hydrogen, alkyl_{(C≤12)}, cycloalkyl_{(C≤12)}, alkenyl_{(C≤12)}, alkynyl_{(C≤12)}, or a substituted version of the last four groups, or -alkanediyl_{(C≤8)}-cycloalkyl_{(C≤12)} or a substituted version of this group;
R₂′ is absent, hydrogen, alkyl_{(C≤12)}, cycloalkyl_{(C≤12)}, alkenyl_{(C≤12)}, alkynyl_{(C≤12)}, or a substituted version of the last four groups, provided that when the bond between carbon atoms 4 and 5 is a double bond then R₂′ is absent;
R₃ is alkyl_{(C≤12)}, aryl_{(C≤12)}, aralkyl_{(C≤12)}, or a substituted version of any of these groups;
R₄ is cycloalkyl_{(C≤18)}, substituted cycloalkyl_{(C≤8)}, heteroaryl_{(C≤18)}, substituted heteroaryl_{(C≤18)}, heterocycloalkyl_{(C≤8)}, substituted heterocycloalkyl_{(C≤8)}, or

—X₁—(CH₂)ₘ—R₄′;
wherein:
   X₁ is NR_{b}, O, or S; wherein:
      R_{b} is hydrogen, alkyl_{(C≤6)}, or substituted alkyl_{(C≤6)}; m is 0, 1, 2, 3, or 4; and
   R₄′ is cycloalkyl_{(C≤18)}, aryl_{(C≤18)}, aralkyl_{(C≤18)}, heteroaryl_{(C≤18)}, heteroaralkyl_{(C≤18)}, heterocycloalkyl_{(C≤8)}, or a substituted version of any of these groups; or

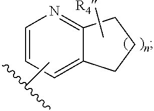

wherein:
   n is 0, 1, 2, 3, or 4; and
   R₄″ is —H, —OH, —F, —Cl, —Br, —I, —NH₂, —NO₂, —CN, —SH, —S(O)₂OH, or —S(O)₂NH₂, or alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; or

—X₂—(CH₂)ₚ—R₄′″;
wherein:
   X₂ is arenediyl_{(C≤12)}, substituted arenediyl_{(C≤12)}, heterocycloalkyldiyl_{(C≤12)}, substituted heterocycloalkyldiyl_{(C≤12)}, heteroarenediyl_{(C≤12)}, or substituted heteroarenediyl_{(C≤12)};
   p is 0, 1, 2, 3, or 4; and
   R₄′″ is alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, alkoxy_{(C≤8)}, acyloxy_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups; and
R₅ is cycloalkoxy_{(C≤12)}, aryl_{(C≤12)}, heteroaryl_{(C≤12)}, or a substituted version of any of the last three groups, or

—OY₁-A₁;
wherein:
   Y₁ is alkanediyl_{(C≤8)} or substituted alkanediyl_{(C≤8)}; and
   A₁ is cycloalkyl_{(C≤8)} or substituted cycloalkyl_{(C≤8)}; or provided that when carbon atoms 4 and 5 are joined by a double bond, then R₂′ and the
   hydrogen atom at carbon atom 5 are absent;
      or a pharmaceutically acceptable salt thereof.

In some embodiments, the bond between carbon atom 1 and carbon atom 2 is an epoxidized double bond. In other embodiments, the bond between carbon atom 1 and carbon atom 2 is a double bond. In some embodiments, the bond between carbon atom 4 and carbon atom 5 is a single bond. In other embodiments, the bond between carbon atom 4 and carbon atom 5 is a double bond. In some embodiments, a is 1. In other embodiments, a is 0. In still other embodiments, a is 2. In some embodiments, R¹ is cyano. In some embodiments, R₂ is alkyl_{(C≤12)}, alkenyl_{(C≤12)}, or a substituted version of either of these groups. In further embodiments, R₂ is alkyl_{(C≤12)} or substituted alkyl_{(C≤12)}. In still further embodiments, R₂ is alkyl_{(C≤12)}, such as methyl, ethyl, or propyl. In some embodiments, R₂ is methyl. In other embodiments, R₂ is substituted alkyl_{(C≤12)}, such as 3-hydroxypropyl. In still other embodiments, R₂ is alkenyl_{(C≤12)} or substituted alkenyl_{(C≤12)}. In further embodiments, R₂ is alkenyl_{(C≤12)}, such as 2-propenyl or 2-methyl-prop-2-enyl. In some embodiments, R₂′ is hydrogen. In other embodiments, R₂′ is alkyl_{(C≤12)} or substituted alkyl_{(C≤12)}. In further embodiments, R₂′ is alkyl_{(C≤12)}, such as methyl. In some embodiments, R₃ is alkyl_{(C≤12)}, aryl_{(C≤12)}, or a substituted version of either of these groups. In further embodiments, R₃ is alkyl_{(C≤12)} or substituted alkyl_{(C≤12)}. In still further embodiments, R₃ is alkyl_{(C≤12)}, such as methyl, propyl, or isopentyl. In some embodiments, R₃ is methyl. In other embodiments, R₃ is aryl_{(C≤12)} or substituted aryl_{(C≤12)}. In further embodiments, R₃ is aryl_{(C≤12)}, such as phenyl.

In some embodiments, R₄ is alkyl_{(C2-8)} or substituted alkyl_{(C2-8)}. In other embodiments, R₄ is aryl_{(C≤18)}, heteroaryl_{(C≤8)}, or a substituted version of either of these groups. In further embodiments, R₄ is heteroaryl_{(C≤18)} or substituted heteroaryl_{(C≤8)}. In still further embodiments, R₄ is a heteroaryl_{(C≤12)} or a substituted heteroaryl_{(C≤12)} group wherein at least one of the heteroatoms in the aromatic ring is a nitrogen atom. In yet further embodiments, R₄ is heteroaryl_{(C≤18)}, such as 3-pyridinyl, 4-pyridinyl, 4-(2-cycylopropyl)-pyridinyl, 5-(2-cycylopropyl)-pyridinyl, 4-(2-morpholino)-pyridinyl, 4-(2-phenyl)-pyridinyl, 3-(5-methyl)-pyridinyl, 3-(6-methyl)-pyridinyl, 4-(2-methyl)-pyridinyl, 4-(3-methyl)-pyridinyl, 3-pyrazolo[1,5-a]pyridinyl, 3-(N-methyl)-pyrrolo[2,3-b]pyridinyl, 5-isoquinlinyl, 2-isoquinlinyl, 1-isoquinolinyl, 4-(2-phenyl)-pyridinyl, 5-(2-phenyl)-pyridinyl, 3-(5-methyl)-pyridinyl, 4-(3-methyl)-pyridinyl, 4-(3,5-dimethyl)-isoxazolyl, 4-(2-methyl)-pyridinyl, 4-(3-methyl)-pyridinyl, 3-(4-methyl)-pyridinyl, 4-(6-methyl)-pyrimidinyl, 6-(4-methyl)-pyrimidinyl, 4-pyridazinyl, 2-quinazolinyl, 4-quinazolinyl, 2-quinolinyl, 3-quinolinyl, 4-quinolinyl, 5-quinolinyl, 6-quinolinyl, 8-quinolinyl, 4-isoquinolinyl, 3-(8-methyl)-quinolinyl, 3-(1-methyl)-quinolinyl, 4-(2-methyl)-quinolinyl, 4-(2-isopropyl)-quinolinyl, 4-(6-methyl)-quinolinyl, 4-(7-methyl)-quinolinyl, 4-(8-methyl)-quinolinyl, 2-(N-methyl)-indolyl, 5-(2,4-dimethyl)-thiazolyl, or 5-(3-methyl)-oxadizolyl. In other embodiments, substituted heteroaryl_{(C≤8)}, such as 4-(2-trifluoromethyl)-pyridinyl, 4-(3-fluoro)-pyridinyl, 4-(2-methoxy)-pyridinyl, 4-(2-hydroxymethyl)-pyridinyl, 4-(2-acetylamino)-pyridinyl, 4-(2-fluoromethyl)-pyridinyl, 4-(2-acetamidylethyl)-pyridinyl, 4-(2-fluoromethyl)-quinolinyl, 4-(2-acetoxymethyl)-quinolinyl, 4-(2-formyl)-quinolinyl, 4-(6-fluoro)-quinolinyl, 4-(7-fluoro)-quinolinyl, 4-(8-fluoro)-quinolinyl, 4-(6,8-difluoro)-quinolinyl, 4-(6-fluoro-2-methyl)-quinolinyl, or 4-(8-fluoro-2-methyl)-quinolinyl. In still other embodiments, R₄ is aryl_{(C≤12)} or substituted aryl_{(C≤12)}. In further embodiments, R₄ is aryl_{(C≤12)}, such as phenyl. In yet other embodiments, R₄ is substituted aryl_{(C≤12)}, such as 2-fluorophenyl or 4-trifluoromethylphenyl. In other embodiments, R₄ is cycloalkyl_{(C≤12)} or substituted cycloalkyl_{(C≤12)}. In further embodiments, R₄ is cycloalkyl_{(C≤12)}, such as cyclohexyl. In still other embodiments, R₄ is:

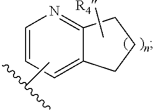

wherein:
n is 0, 1, 2, 3, or 4; and
R₄″ is —H, —OH, —F, —Cl, —Br, —I, —NH₂, —NO₂, —CN, —SH, —S(O)₂OH, or —S(O)₂NH₂, or alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, alkylamino_{(C≤8)}, dialkylamino_{(C≤8)}, —C(O)-alkoxy_{(C≤8)}, —C(O)-alkylamino_{(C≤8)}, —C(O)-dialkyl-amino_{(C≤8)}, alkylsulfonyl_{(C≤8)}, arylsulfonyl_{(C≤8)}, alkoxysulfonyl_{(C≤8)}, or a substituted version of any of these groups.
   In further embodiments, R₄ is:

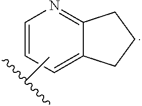

In still further embodiments, R₄ is:

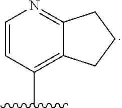 [SMILES: *c1ccnc2c1CCC2]

In yet other embodiments, R₄ is heterocycloalkyl_{(C≤18)} or substituted heterocycloalkyl_{(C≤18)}. In further embodiments, R₄ is heterocycloalkyl_{(C≤12)}, such as morpholinyl, 4-piperidinyl, 3-(5-methyl-)1,2,3,6-tetrahydropyridinyl, or 4-N-methylpiperazinyl. In other embodiments, R₄ is substituted heterocycloalkyl_{(C≤12)}, such as N-t-butyloxycarbonyl-4-piperidinyl, N-acetyl-4-piperidinyl, N-t-butyloxycarbonyl-5-methyl-1,2,3,6-tetrahydropyridinyl, N-acetyl-5-methyl-1,2,3,6-tetrahydropyridinyl, or 4-N-acetylpiperazinyl. In still other embodiments, R₄ is hydrogen. In yet other embodiments, R₄ is —X₁—(CH₂)ₘ—R₄′; wherein:
X₁ is NR_{b}, O, or S; wherein:
   R_{b} is hydrogen, alkyl_{(C≤6)}, or substituted alkyl_{(C≤6)};
m is 0, 1, 2, 3, or 4; and
R₄′ is alkyl_{(C≤12)}, cycloalkyl_{(C≤18)}, aryl_{(C≤18)}, aralkyl_{(C≤18)}, heteroaryl_{(C≤18)}, heteroaralkyl_{(C≤18)}, heterocycloalkyl_{(C≤18)}, or a substituted version of any of these groups, provided that when X₁ is 0, then R₄′ is not methyl.
   In some embodiments, X₁ is NR_{b}, wherein: R_{b} is hydrogen, alkyl_{(C≤6)}, or substituted alkyl_{(C≤6)}. In further embodiments, R_{b} is hydrogen. In some embodiments, m is 0 or 1. In some embodiments, m is 0. In some embodiments, R₄′ is cycloalkyl_{(C≤18)}, aryl_{(C≤18)}, aralkyl_{(C≤18)}, heteroaryl_{(C≤18)}, heteroaralkyl_{(C≤18)}, heterocycloalkyl_{(C≤18)}, or a substituted version of any of these groups. In further embodiments, R₄′ is heteroaryl_{(C≤12)} or substituted heteroaryl_{(C≤12)}. In still further embodiments, R₄′ is heteroaryl_{(C≤12)}, such as 4-pyridinyl. In other embodiments, R₄ is amino. In still other embodiments, R₄ is amido_{(C≤12)} or substituted amido_{(C≤12)}. In further embodiments, R₄ is amido_{(C≤12)}, such as:

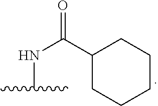 [SMILES: *NC(=O)C1CCCCC1]

In other embodiments, R₄ is —X₂—(CH₂)ₚ—R₄′; wherein:
X₂ is arenediyl_{(C≤12)}, substituted arenediyl_{(C≤12)}, heterocycloalkanediyl_{(C≤12)}, substituted heterocycloalkanediyl_{(C≤12)}, heteroarenediyl_{(C≤12)}, or substituted heteroarenediyl_{(C≤12)};
p is 0, 1, 2, 3, or 4; and
R₄′ is alkyl_{(C≤8)}, cycloalkyl_{(C≤8)}, aryl_{(C≤8)}, heteroaryl_{(C≤8)}, heterocycloalkyl_{(C≤8)}, acyl_{(C≤8)}, amido_{(C≤8)}, alkoxy_{(C≤8)}, acyloxy_{(C≤8)}, —C(O)-alkoxy_{(C≤8)}, —C(O)-alkylamino_{(C≤8)}, —C(O)-dialkylamino_{(C≤8)}, alkylsulfonyl_{(C≤8)}, arylsulfonyl_{(C≤8)}, alkoxysulfonyl_{(C≤8)}, or a substituted version of any of these groups.

In some embodiments, X₂ is heteroarenediyl_{(C≤12)} or substituted heteroarenediyl_{(C≤12)}. In further embodiments, X₂ is heteroarenediyl_{(C≤12)}, such as pyridin-2,4-diyl or pyridine-2,5-diyl. In still other embodiments, X₂ is heterocycloalkanediyl_{(C≤12)} or substituted heterocycloalkanediyl_{(C≤12)}. In further embodiments, X₂ is heterocycloalkanediyl_{(C≤12)}, such as piperidin-1,4-diyl, piperazin-1,4-diyl, or 1,2,3,6-tetrahydropiperidin-1,5-diyl. In some embodiments, p is 0, 1, or 2. In some embodiments, p is 0. In other embodiments, p is 1. In still other embodiments, p is 2. In some embodiments, R₄′″ is acyl_{(C≤8)} or substituted acyl_{(C≤8)}. In further embodiments, R₄′″ is acyl_{(C≤8)}, such as acetyl. In yet other embodiments, R₄′″ is substituted acyl_{(C≤8)}. In some embodiments, R₄′″ is carboxy. In other embodiments, R₄′″ is amido_{(C≤8)} or substituted amido_{(C≤8)}. In further embodiments, R₄′″ is amido_{(C≤8)}, such as acetamidyl. In still other embodiments, R₄′″ is cycloalkyl_{(C≤8)} or substituted cycloalkyl_{(C≤8)}. In further embodiments, R₄′″ is cycloalkyl_{(C≤8)}, such as cyclopropyl. In yet other embodiments, R₄′″ is alkylsulfonyl_{(C≤8)} or substituted alkylsulfonyl_{(C≤8)}. In further embodiments, R₄′″ is alkylsulfonyl_{(C≤8)}, such as —S(O)₂CH₃ or —S(O)₂CH₂CH₃. In other embodiments, R₄′″ is —C(O)-alkoxy_{(C≤8)}, such as —C(O)OEt. In still other embodiments, R₄′″ is —C(O)-dialkylamino_{(C≤8)}, such as —C(O)NMe₂.

In some embodiments, R₅ is aryl_{(C≤12)}, heteroaryl_{(C≤12)}, or a substituted version of either of these groups. In further embodiments, R₅ is aryl_{(C≤12)} or substituted aryl_{(C≤12)}. In still further embodiments, R₅ is aryl_{(C≤12)}, such as phenyl, 4-methylphenyl, 3-isopropylphenyl, 4-isopropylphenyl, 1,3-biphenyl, or 1,4-biphenyl. In some embodiments, R₅ further comprises one or more fluorine atoms. In some embodiments, R₅ is substituted aryl_{(C≤12)}, such as R₅ is 2-fluorophenyl, 3-fluorophenyl, 4-fluorophenyl, 2,4-difluorophenyl, 4-hydroxymethylphenyl, 3-trifluoromethylphenyl, 4-trifluoromethylphenyl, 4-methoxyphenyl, 4-chlorophenyl, or 3,4-dichlorophenyl. In other embodiments, R₅ is cycloalkyl_{(C≤12)}, such as cyclopropyl, or substituted cycloalkyl_{(C≤12)}. In still other embodiments, R₅ is cycloalkoxy_{(C≤12)}, such as cyclobutyloxy, cyclopentyloxy, or cyclohexyloxy, or substituted cycloalkoxy_{(C≤12)}. In yet other embodiments, R₅ is alkylamino_{(C≤12)}, dialkylamino_{(C≤12)}, or a substituted version of either of these groups. In further embodiments, R₅ is dialkylamino_{(C≤12)} or substituted dialkylamino_{(C≤12)}. In still further embodiments, R₅ is dialkylamino(c s), such as dimethylamino, or substituted dialkylamino_{(C≤8)}. In other embodiments, R₅ is alkylsulfonylamino_{(C≤12)} or substituted alkylsulfonylamino_{(C≤12)}. In further embodiments, R₆ is methylsulfonylamino. In still other embodiments, R₅ is —OY₁-A;

wherein:
Y₁ is alkanediyl(c s) or substituted alkanediyl(c s); and
A₁ is cycloalkyl(c s) or substituted cycloalkyl(c s).
   In some embodiments, Y₁ is methylene. In some embodiments, A₁ is cyclobutyl. In some embodiments, R₅ is:

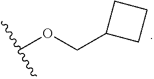 [SMILES: *OCC1CCC1]

In other embodiments, R₅ is —Y₂—C(O)NRₑ-A₂; wherein:
Y₂ is arenediyl(c s) or substituted arenediyl(c s);
R_{c} is hydrogen, alkyl_{(C≤6)}, or substituted alkyl_{(C≤6)}; and
A₂ is aralkyl_{(C≤12)} or substituted aralkyl_{(C≤12)}.

In some embodiments, Y₂ is arenediyl(c s), such as benzenediyl. In some embodiments, R_{c} is alkyl_{(C≤6)}, such as methyl. In some embodiments, A₂ is aralkyl_{(C≤12)}, such as benzyl. In still other embodiments, R₅ is heteroaryl_{(C≤12)} or substituted heteroaryl_{(C≤12)}. In further embodiments, R₅ is heteroaryl_{(C≤12)}, such as 5-(3-methyl)-oxadiazolyl, 4-(3,5-dimethyl)-isoxazolyl, furanyl, benzofuranyl, 2-thiazolyl, 5-(2-methyl)-furanyl, 3-pyridinyl, or 4-pyridinyl. In yet other embodiments, R₅ is hydroxy. In other embodiments, R₅ is —OS(O)₂C₆H₄CH₃. In still other embodiments, R₅ is heterocycloalkyl_{(C≤12)} or substituted heterocycloalkyl_{(C≤12)}. In further embodiments, R₅ is heterocycloalkyl_{(C≤12)}, such as pyrrolidinyl. In yet other embodiments, R₅ is alkoxy_{(C≤12)} or substituted alkoxy_{(C≤12)}. In further embodiments, R₅ is alkoxy_{(C≤12)}, such as methoxy or isopropoxy. In other embodiments, R₅ is aralkyl_{(C≤12)} or substituted aralkyl_{(C≤12)}. In further embodiments, R₅ is aralkyl_{(C≤12)}, such as benzyl.

In some embodiments, the compound is further defined as:

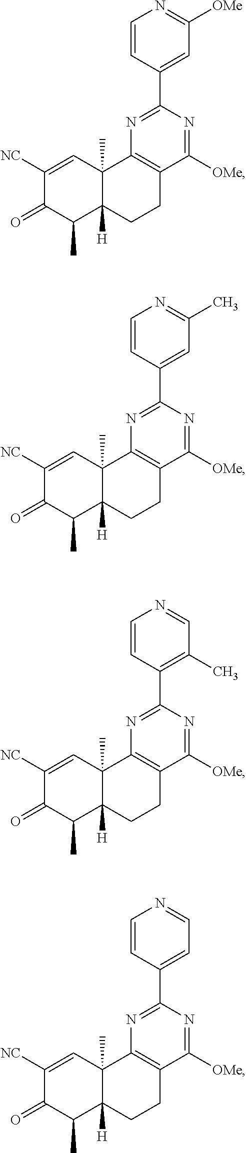 [SMILES: COc1cc(-c2nc(OC)c3c(n2)[C@]2(C)C=C(C#N)C(=O)[C@H](C)[C@H]2CC3)ccn1 ; COc1nc(-c2ccnc(C)c2)nc2c1CC[C@@H]1[C@@H](C)C(=O)C(C#N)=C[C@@]21C ; COc1nc(-c2ccncc2C)nc2c1CC[C@@H]1[C@@H](C)C(=O)C(C#N)=C[C@@]21C ; COc1nc(-c2ccncc2)nc2c1CC[C@@H]1[C@@H](C)C(=O)C(C#N)=C[C@@]21C]

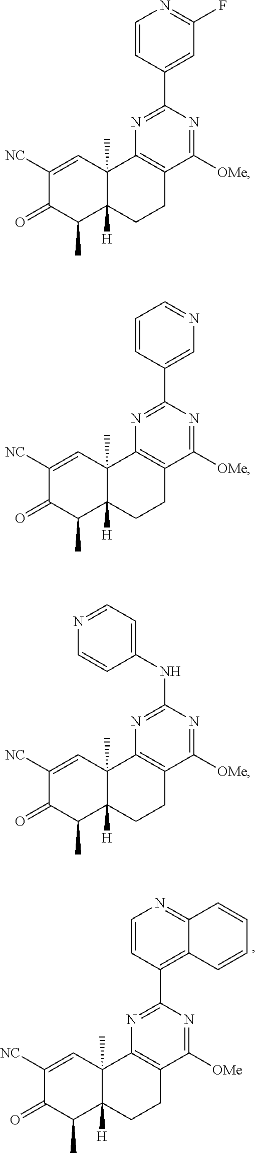 [SMILES: COc1nc(-c2ccnc(F)c2)nc2c1CC[C@@H]1[C@@H](C)C(=O)C(C#N)=C[C@@]21C ; COc1nc(-c2cccnc2)nc2c1CC[C@@H]1[C@@H](C)C(=O)C(C#N)=C[C@@]21C ; COc1nc(Nc2ccncc2)nc2c1CC[C@@H]1[C@@H](C)C(=O)C(C#N)=C[C@@]21C ; COc1nc(-c2ccnc3ccccc23)nc2c1CC[C@@H]1[C@@H](C)C(=O)C(C#N)=C[C@@]21C]

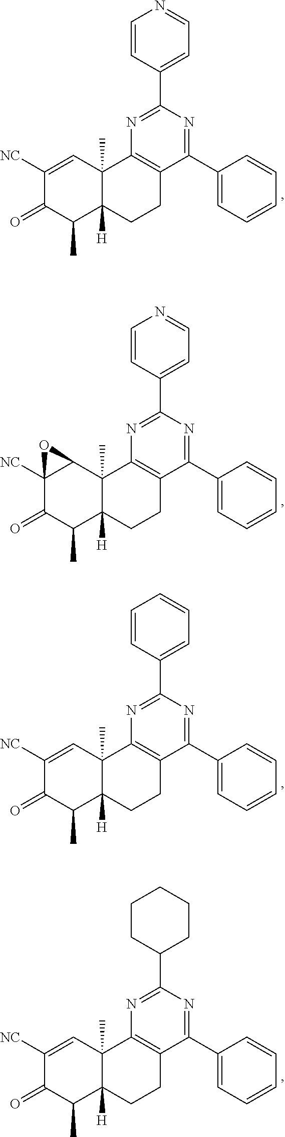 [SMILES: C[C@H]1C(=O)C(C#N)=C[C@@]2(C)c3nc(-c4ccncc4)nc(-c4ccccc4)c3CC[C@H]12 ; C[C@H]1C(=O)[C@@]2(C#N)O[C@H]2[C@@]2(C)c3nc(-c4ccncc4)nc(-c4ccccc4)c3CC[C@H]12 ; C[C@H]1C(=O)C(C#N)=C[C@@]2(C)c3nc(-c4ccccc4)nc(-c4ccccc4)c3CC[C@H]12 ; C[C@H]1C(=O)C(C#N)=C[C@@]2(C)c3nc(C4CCCCC4)nc(-c4ccccc4)c3CC[C@H]12]

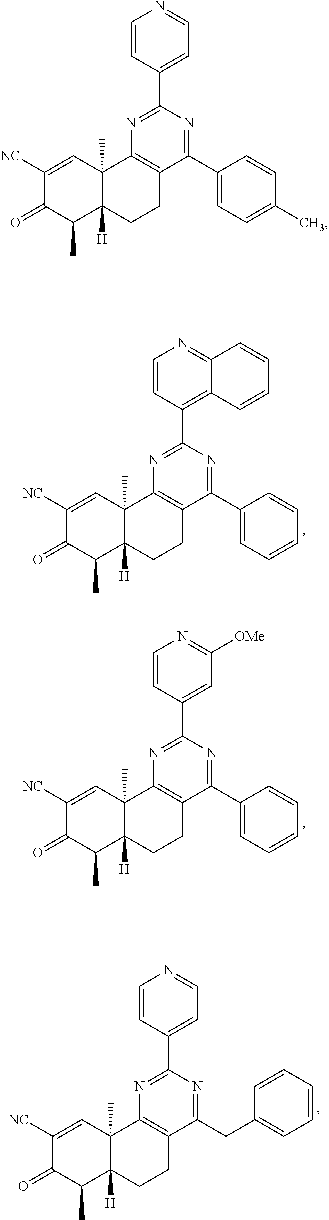 [SMILES: Cc1ccc(-c2nc(-c3ccncc3)nc3c2CC[C@@H]2[C@@H](C)C(=O)C(C#N)=C[C@@]32C)cc1 ; C[C@H]1C(=O)C(C#N)=C[C@@]2(C)c3nc(-c4ccnc5ccccc45)nc(-c4ccccc4)c3CC[C@H]12 ; COc1cc(-c2nc(-c3ccccc3)c3c(n2)[C@]2(C)C=C(C#N)C(=O)[C@H](C)[C@H]2CC3)ccn1 ; C[C@H]1C(=O)C(C#N)=C[C@@]2(C)c3nc(-c4ccncc4)nc(Cc4ccccc4)c3CC[C@H]12]

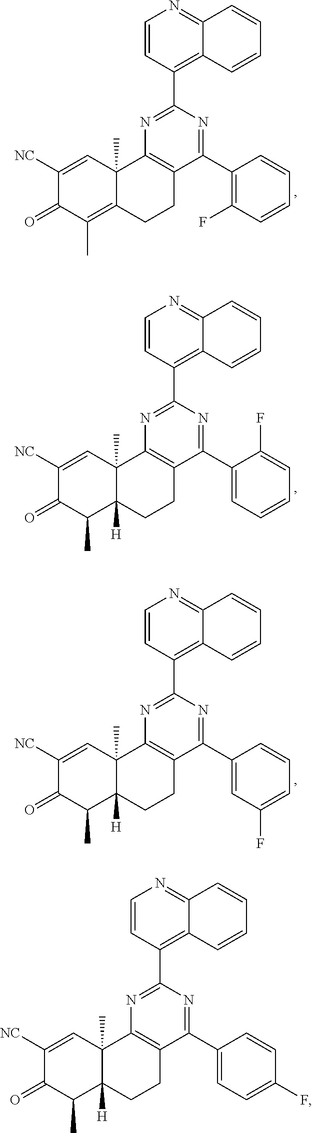 [SMILES: CC1=C2CCc3c(-c4ccccc4F)nc(-c4ccnc5ccccc45)nc3[C@]2(C)C=C(C#N)C1=O ; C[C@H]1C(=O)C(C#N)=C[C@@]2(C)c3nc(-c4ccnc5ccccc45)nc(-c4ccccc4F)c3CC[C@H]12 ; C[C@H]1C(=O)C(C#N)=C[C@@]2(C)c3nc(-c4ccnc5ccccc45)nc(-c4cccc(F)c4)c3CC[C@H]12 ; C[C@H]1C(=O)C(C#N)=C[C@@]2(C)c3nc(-c4ccnc5ccccc45)nc(-c4ccc(F)cc4)c3CC[C@H]12]

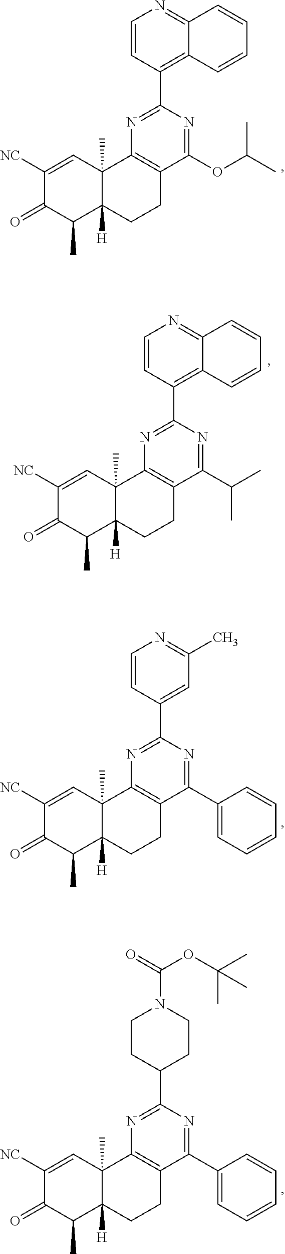 [SMILES: CC(C)Oc1nc(-c2ccnc3ccccc23)nc2c1CC[C@@H]1[C@@H](C)C(=O)C(C#N)=C[C@@]21C ; CC(C)c1nc(-c2ccnc3ccccc23)nc2c1CC[C@@H]1[C@@H](C)C(=O)C(C#N)=C[C@@]21C ; Cc1cc(-c2nc(-c3ccccc3)c3c(n2)[C@]2(C)C=C(C#N)C(=O)[C@H](C)[C@H]2CC3)ccn1 ; C[C@H]1C(=O)C(C#N)=C[C@@]2(C)c3nc(C4CCN(C(=O)OC(C)(C)C)CC4)nc(-c4ccccc4)c3CC[C@H]12]

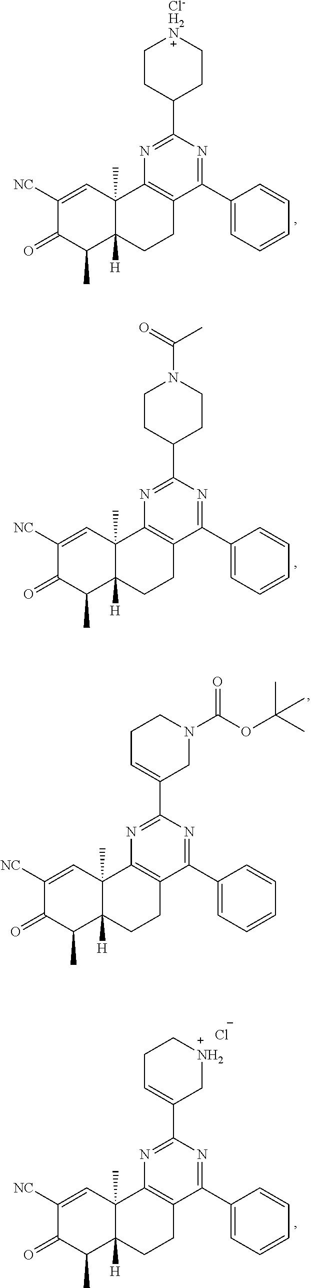

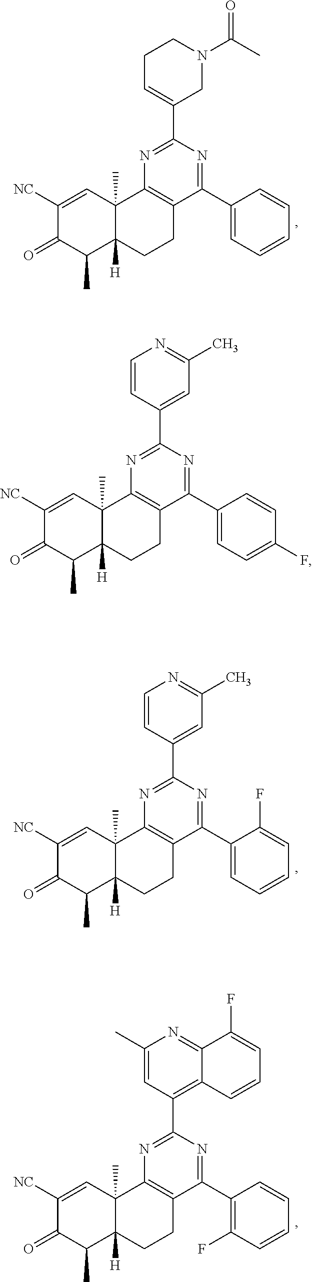 [SMILES: CC(=O)N1CCC=C(c2nc(-c3ccccc3)c3c(n2)[C@]2(C)C=C(C#N)C(=O)[C@H](C)[C@H]2CC3)C1 ; Cc1cc(-c2nc(-c3ccc(F)cc3)c3c(n2)[C@]2(C)C=C(C#N)C(=O)[C@H](C)[C@H]2CC3)ccn1 ; Cc1cc(-c2nc(-c3ccccc3F)c3c(n2)[C@]2(C)C=C(C#N)C(=O)[C@H](C)[C@H]2CC3)ccn1 ; Cc1cc(-c2nc(-c3ccccc3F)c3c(n2)[C@]2(C)C=C(C#N)C(=O)[C@H](C)[C@H]2CC3)c2cccc(F)c2n1]

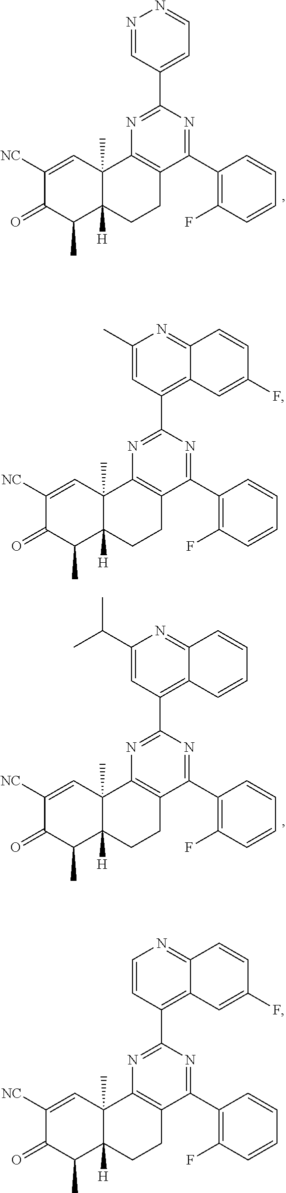

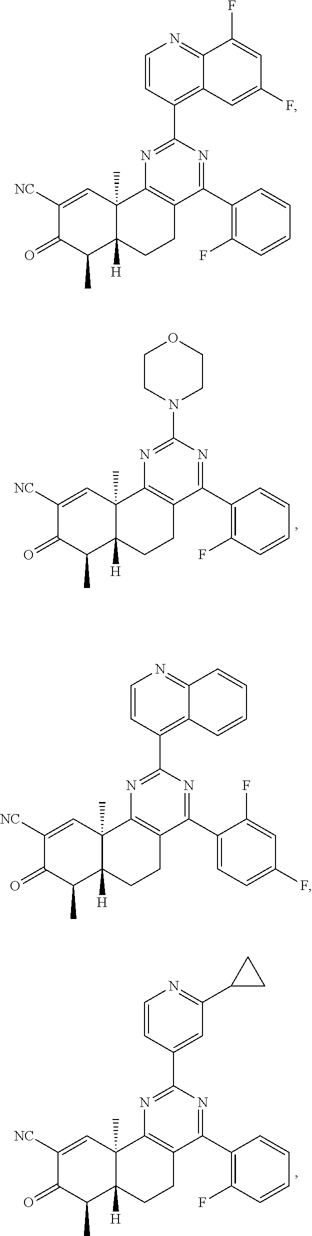 [SMILES: C[C@H]1C(=O)C(C#N)=C[C@@]2(C)c3nc(-c4ccnc5c(F)cc(F)cc45)nc(-c4ccccc4F)c3CC[C@H]12 ; C[C@H]1C(=O)C(C#N)=C[C@@]2(C)c3nc(N4CCOCC4)nc(-c4ccccc4F)c3CC[C@H]12 ; C[C@H]1C(=O)C(C#N)=C[C@@]2(C)c3nc(-c4ccnc5ccccc45)nc(-c4ccc(F)cc4F)c3CC[C@H]12 ; C[C@H]1C(=O)C(C#N)=C[C@@]2(C)c3nc(-c4ccnc(C5CC5)c4)nc(-c4ccccc4F)c3CC[C@H]12]

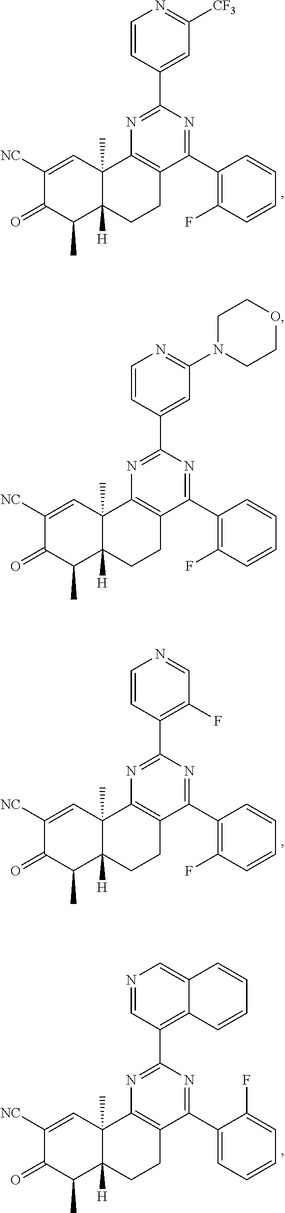 [SMILES: C[C@H]1C(=O)C(C#N)=C[C@@]2(C)c3nc(-c4ccnc(C(F)(F)F)c4)nc(-c4ccccc4F)c3CC[C@H]12 ; C[C@H]1C(=O)C(C#N)=C[C@@]2(C)c3nc(-c4ccnc(N5CCOCC5)c4)nc(-c4ccccc4F)c3CC[C@H]12 ; C[C@H]1C(=O)C(C#N)=C[C@@]2(C)c3nc(-c4ccncc4F)nc(-c4ccccc4F)c3CC[C@H]12 ; C[C@H]1C(=O)C(C#N)=C[C@@]2(C)c3nc(-c4cncc5ccccc45)nc(-c4ccccc4F)c3CC[C@H]12]

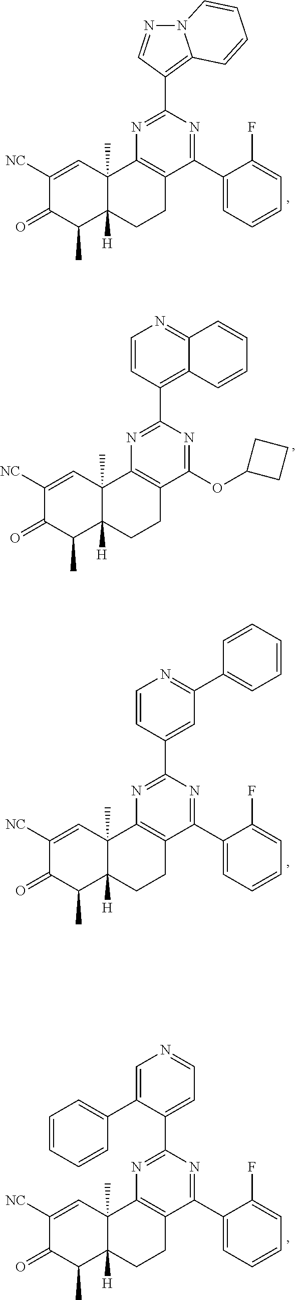 [SMILES: C[C@H]1C(=O)C(C#N)=C[C@@]2(C)c3nc(-c4cnn5ccccc45)nc(-c4ccccc4F)c3CC[C@H]12 ; C[C@H]1C(=O)C(C#N)=C[C@@]2(C)c3nc(-c4ccnc5ccccc45)nc(OC4CCC4)c3CC[C@H]12 ; C[C@H]1C(=O)C(C#N)=C[C@@]2(C)c3nc(-c4ccnc(-c5ccccc5)c4)nc(-c4ccccc4F)c3CC[C@H]12 ; C[C@H]1C(=O)C(C#N)=C[C@@]2(C)c3nc(-c4ccncc4-c4ccccc4)nc(-c4ccccc4F)c3CC[C@H]12]

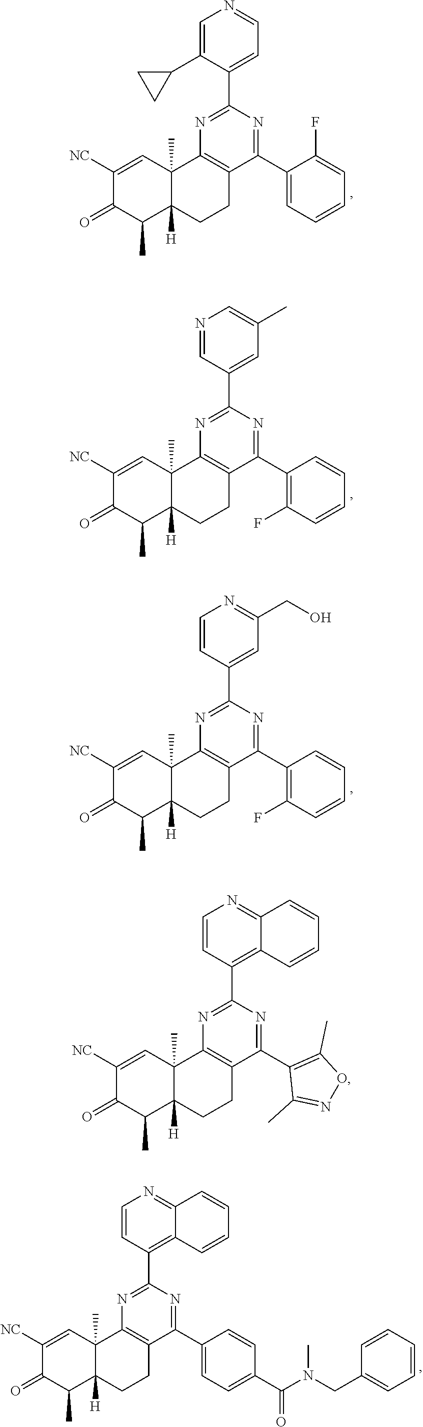

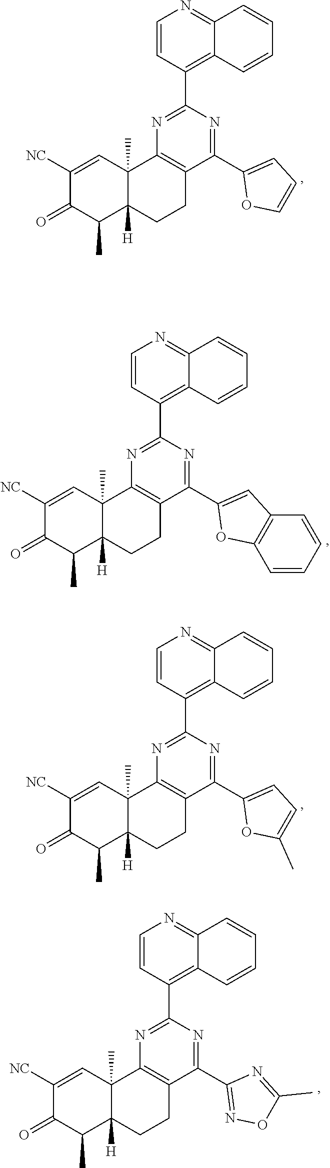 [SMILES: C[C@H]1C(=O)C(C#N)=C[C@@]2(C)c3nc(-c4ccnc5ccccc45)nc(-c4ccco4)c3CC[C@H]12 ; C[C@H]1C(=O)C(C#N)=C[C@@]2(C)c3nc(-c4ccnc5ccccc45)nc(-c4cc5ccccc5o4)c3CC[C@H]12 ; Cc1ccc(-c2nc(-c3ccnc4ccccc34)nc3c2CC[C@@H]2[C@@H](C)C(=O)C(C#N)=C[C@@]32C)o1 ; Cc1nc(-c2nc(-c3ccnc4ccccc34)nc3c2CC[C@@H]2[C@@H](C)C(=O)C(C#N)=C[C@@]32C)no1]

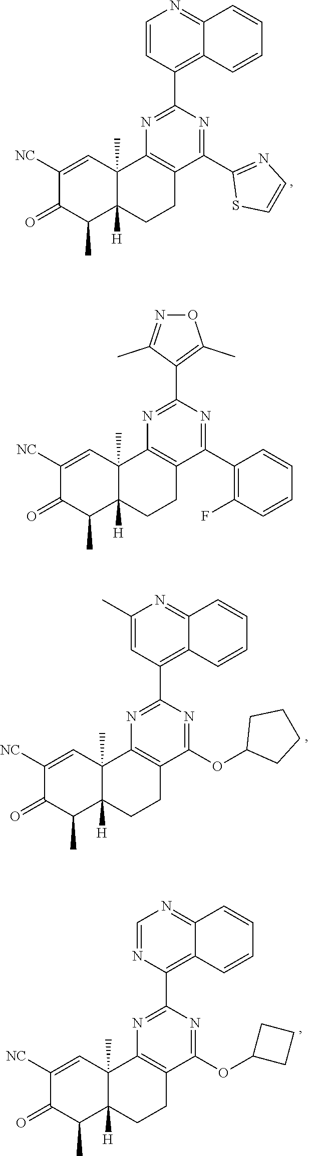 [SMILES: C[C@H]1C(=O)C(C#N)=C[C@@]2(C)c3nc(-c4ccnc5ccccc45)nc(-c4nccs4)c3CC[C@H]12 ; Cc1noc(C)c1-c1nc(-c2ccccc2F)c2c(n1)[C@]1(C)C=C(C#N)C(=O)[C@H](C)[C@H]1CC2 ; Cc1cc(-c2nc(OC3CCCC3)c3c(n2)[C@]2(C)C=C(C#N)C(=O)[C@H](C)[C@H]2CC3)c2ccccc2n1 ; C[C@H]1C(=O)C(C#N)=C[C@@]2(C)c3nc(-c4ncnc5ccccc45)nc(OC4CCC4)c3CC[C@H]12]

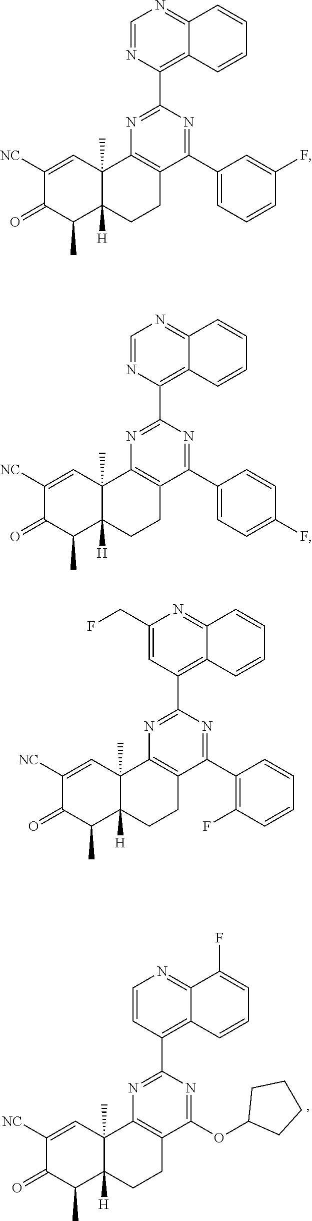 [SMILES: C[C@H]1C(=O)C(C#N)=C[C@@]2(C)c3nc(-c4ncnc5ccccc45)nc(-c4cccc(F)c4)c3CC[C@H]12 ; C[C@H]1C(=O)C(C#N)=C[C@@]2(C)c3nc(-c4ncnc5ccccc45)nc(-c4ccc(F)cc4)c3CC[C@H]12 ; C[C@H]1C(=O)C(C#N)=C[C@@]2(C)c3nc(-c4cc(CF)nc5ccccc45)nc(-c4ccccc4F)c3CC[C@H]12 ; C[C@H]1C(=O)C(C#N)=C[C@@]2(C)c3nc(-c4ccnc5c(F)cccc45)nc(OC4CCCC4)c3CC[C@H]12]

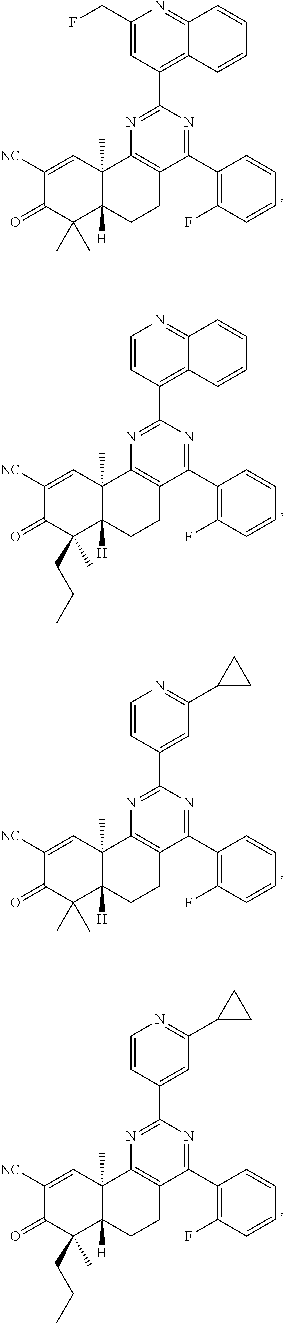 [SMILES: CC1(C)C(=O)C(C#N)=C[C@@]2(C)c3nc(-c4cc(CF)nc5ccccc45)nc(-c4ccccc4F)c3CC[C@H]12 ; CCC[C@@]1(C)C(=O)C(C#N)=C[C@@]2(C)c3nc(-c4ccnc5ccccc45)nc(-c4ccccc4F)c3CC[C@H]12 ; CC1(C)C(=O)C(C#N)=C[C@@]2(C)c3nc(-c4ccnc(C5CC5)c4)nc(-c4ccccc4F)c3CC[C@H]12 ; CCC[C@@]1(C)C(=O)C(C#N)=C[C@@]2(C)c3nc(-c4ccnc(C5CC5)c4)nc(-c4ccccc4F)c3CC[C@H]12]

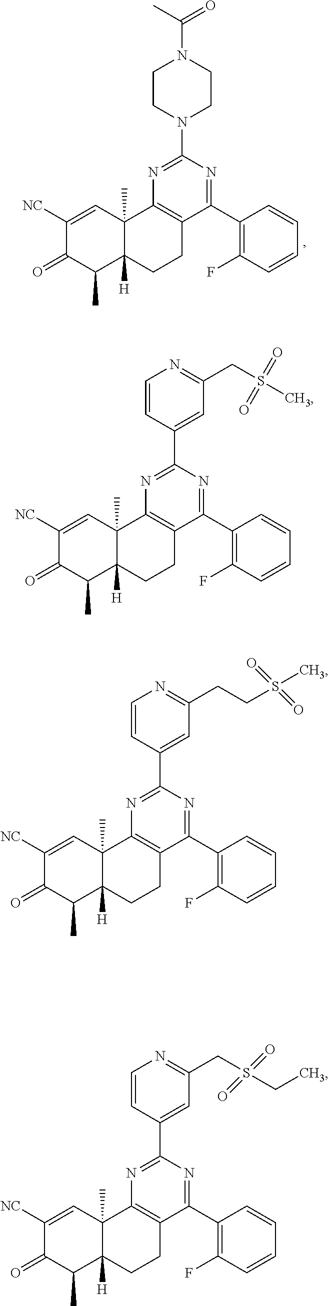 [SMILES: CC(=O)N1CCN(c2nc(-c3ccccc3F)c3c(n2)[C@]2(C)C=C(C#N)C(=O)[C@H](C)[C@H]2CC3)CC1 ; C[C@H]1C(=O)C(C#N)=C[C@@]2(C)c3nc(-c4ccnc(CS(C)(=O)=O)c4)nc(-c4ccccc4F)c3CC[C@H]12 ; C[C@H]1C(=O)C(C#N)=C[C@@]2(C)c3nc(-c4ccnc(CCS(C)(=O)=O)c4)nc(-c4ccccc4F)c3CC[C@H]12 ; CCS(=O)(=O)Cc1cc(-c2nc(-c3ccccc3F)c3c(n2)[C@]2(C)C=C(C#N)C(=O)[C@H](C)[C@H]2CC3)ccn1]

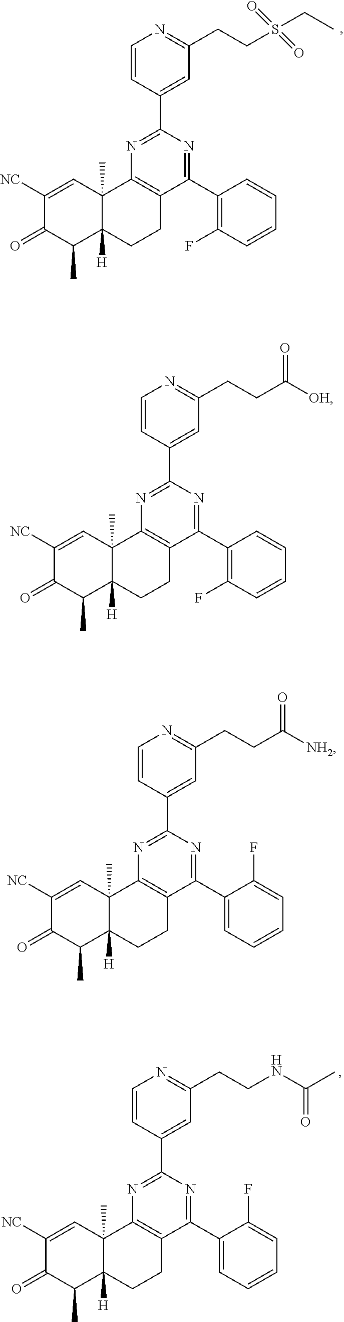 [SMILES: CCS(=O)(=O)CCc1cc(-c2nc(-c3ccccc3F)c3c(n2)[C@]2(C)C=C(C#N)C(=O)[C@H](C)[C@H]2CC3)ccn1 ; C[C@H]1C(=O)C(C#N)=C[C@@]2(C)c3nc(-c4ccnc(CCC(=O)O)c4)nc(-c4ccccc4F)c3CC[C@H]12 ; C[C@H]1C(=O)C(C#N)=C[C@@]2(C)c3nc(-c4ccnc(CCC(N)=O)c4)nc(-c4ccccc4F)c3CC[C@H]12 ; CC(=O)NCCc1cc(-c2nc(-c3ccccc3F)c3c(n2)[C@]2(C)C=C(C#N)C(=O)[C@H](C)[C@H]2CC3)ccn1]

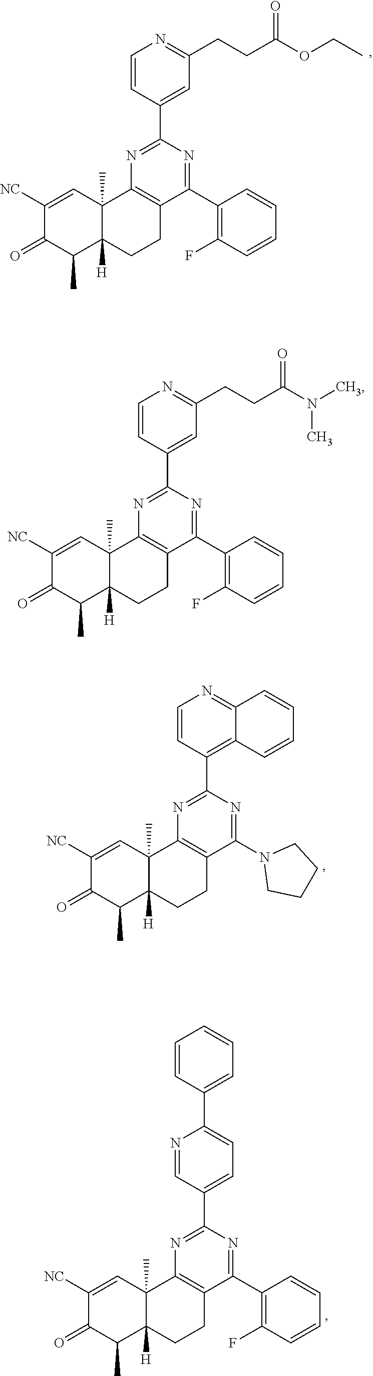 [SMILES: CCOC(=O)CCc1cc(-c2nc(-c3ccccc3F)c3c(n2)[C@]2(C)C=C(C#N)C(=O)[C@H](C)[C@H]2CC3)ccn1 ; C[C@H]1C(=O)C(C#N)=C[C@@]2(C)c3nc(-c4ccnc(CCC(=O)N(C)C)c4)nc(-c4ccccc4F)c3CC[C@H]12 ; C[C@H]1C(=O)C(C#N)=C[C@@]2(C)c3nc(-c4ccnc5ccccc45)nc(N4CCCC4)c3CC[C@H]12 ; C[C@H]1C(=O)C(C#N)=C[C@@]2(C)c3nc(-c4ccc(-c5ccccc5)nc4)nc(-c4ccccc4F)c3CC[C@H]12]

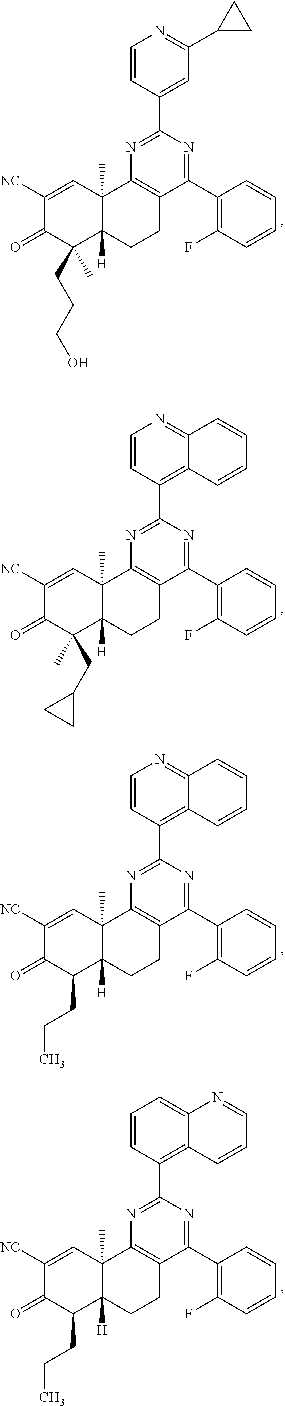 [SMILES: C[C@]1(CCCO)C(=O)C(C#N)=C[C@@]2(C)c3nc(-c4ccnc(C5CC5)c4)nc(-c4ccccc4F)c3CC[C@H]12 ; C[C@]1(CC2CC2)C(=O)C(C#N)=C[C@@]2(C)c3nc(-c4ccnc5ccccc45)nc(-c4ccccc4F)c3CC[C@H]12 ; CCC[C@H]1C(=O)C(C#N)=C[C@@]2(C)c3nc(-c4ccnc5ccccc45)nc(-c4ccccc4F)c3CC[C@H]12 ; CCC[C@H]1C(=O)C(C#N)=C[C@@]2(C)c3nc(-c4cccc5ncccc45)nc(-c4ccccc4F)c3CC[C@H]12]

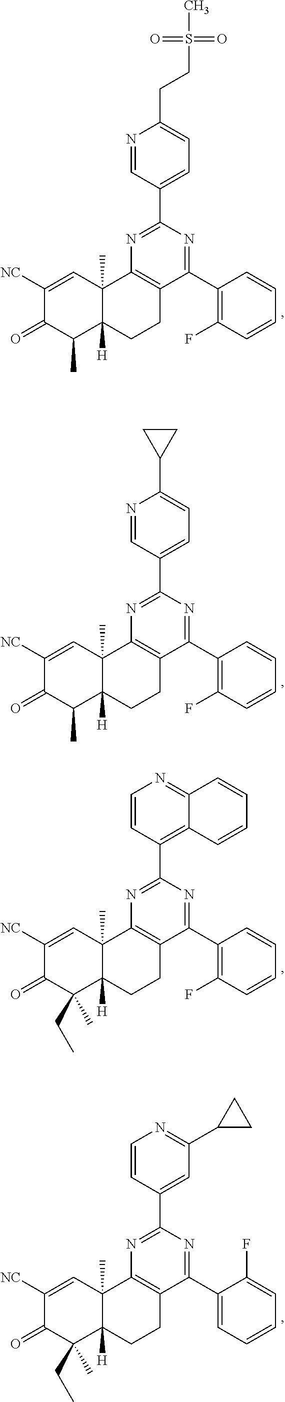 [SMILES: C[C@H]1C(=O)C(C#N)=C[C@@]2(C)c3nc(-c4ccc(CCS(C)(=O)=O)nc4)nc(-c4ccccc4F)c3CC[C@H]12 ; C[C@H]1C(=O)C(C#N)=C[C@@]2(C)c3nc(-c4ccc(C5CC5)nc4)nc(-c4ccccc4F)c3CC[C@H]12 ; CC[C@@]1(C)C(=O)C(C#N)=C[C@@]2(C)c3nc(-c4ccnc5ccccc45)nc(-c4ccccc4F)c3CC[C@H]12 ; CC[C@@]1(C)C(=O)C(C#N)=C[C@@]2(C)c3nc(-c4ccnc(C5CC5)c4)nc(-c4ccccc4F)c3CC[C@H]12]

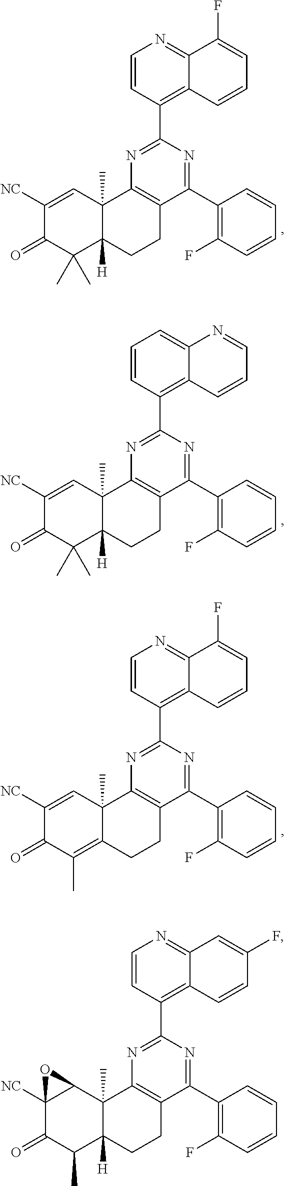 [SMILES: CC1(C)C(=O)C(C#N)=C[C@@]2(C)c3nc(-c4ccnc5c(F)cccc45)nc(-c4ccccc4F)c3CC[C@H]12 ; CC1(C)C(=O)C(C#N)=C[C@@]2(C)c3nc(-c4cccc5ncccc45)nc(-c4ccccc4F)c3CC[C@H]12 ; CC1=C2CCc3c(-c4ccccc4F)nc(-c4ccnc5c(F)cccc45)nc3[C@]2(C)C=C(C#N)C1=O ; C[C@H]1C(=O)[C@@]2(C#N)O[C@H]2[C@@]2(C)c3nc(-c4ccnc5cc(F)ccc45)nc(-c4ccccc4F)c3CC[C@H]12]

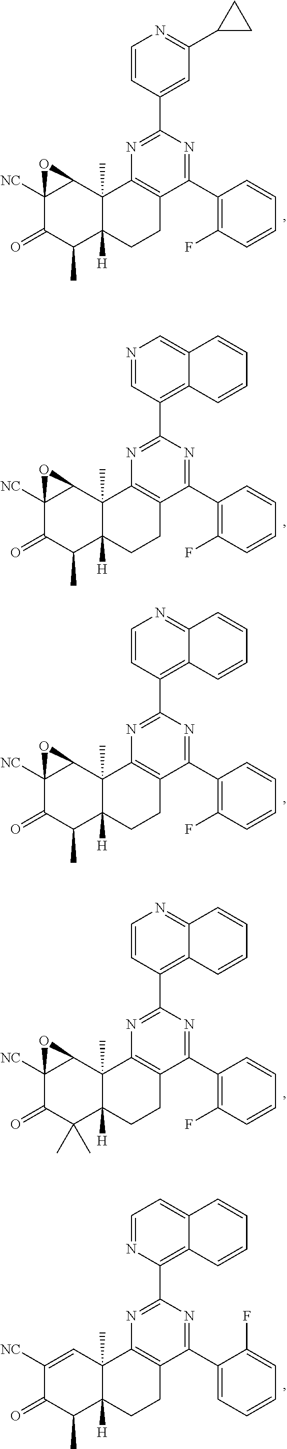 [SMILES: C[C@H]1C(=O)[C@@]2(C#N)O[C@H]2[C@@]2(C)c3nc(-c4ccnc(C5CC5)c4)nc(-c4ccccc4F)c3CC[C@H]12 ; C[C@H]1C(=O)[C@@]2(C#N)O[C@H]2[C@@]2(C)c3nc(-c4cncc5ccccc45)nc(-c4ccccc4F)c3CC[C@H]12 ; C[C@H]1C(=O)[C@@]2(C#N)O[C@H]2[C@@]2(C)c3nc(-c4ccnc5ccccc45)nc(-c4ccccc4F)c3CC[C@H]12 ; CC1(C)C(=O)[C@@]2(C#N)O[C@H]2[C@@]2(C)c3nc(-c4ccnc5ccccc45)nc(-c4ccccc4F)c3CC[C@H]12 ; C[C@H]1C(=O)C(C#N)=C[C@@]2(C)c3nc(-c4nccc5ccccc45)nc(-c4ccccc4F)c3CC[C@H]12]

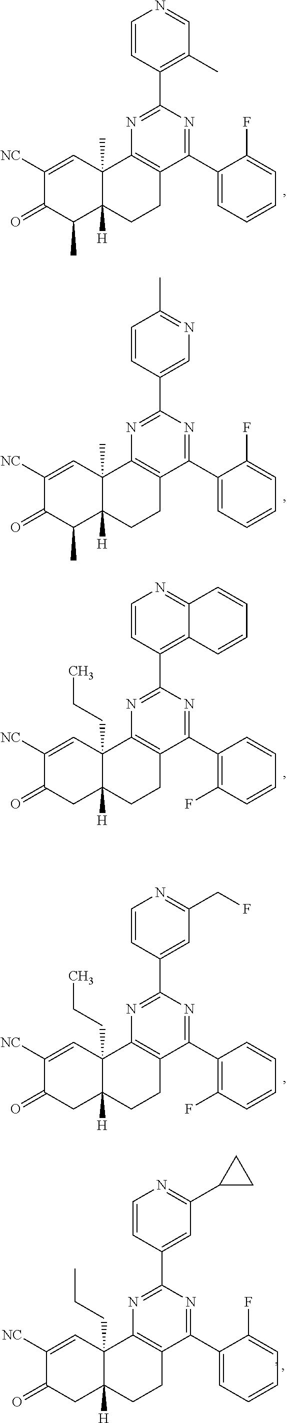 [SMILES: Cc1cnccc1-c1nc(-c2ccccc2F)c2c(n1)[C@]1(C)C=C(C#N)C(=O)[C@H](C)[C@H]1CC2 ; Cc1ccc(-c2nc(-c3ccccc3F)c3c(n2)[C@]2(C)C=C(C#N)C(=O)[C@H](C)[C@H]2CC3)cn1 ; CCC[C@@]12C=C(C#N)C(=O)C[C@H]1CCc1c(-c3ccccc3F)nc(-c3ccnc4ccccc34)nc12 ; CCC[C@@]12C=C(C#N)C(=O)C[C@H]1CCc1c(-c3ccccc3F)nc(-c3ccnc(CF)c3)nc12 ; CCC[C@@]12C=C(C#N)C(=O)C[C@H]1CCc1c(-c3ccccc3F)nc(-c3ccnc(C4CC4)c3)nc12]

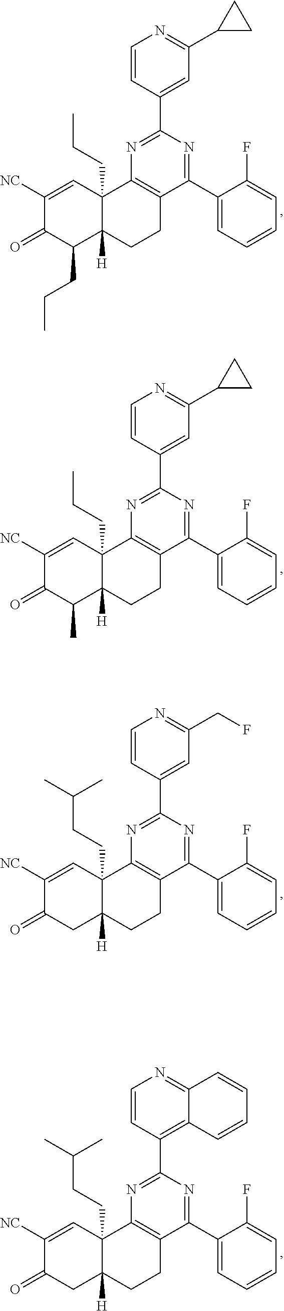 [SMILES: CCC[C@H]1C(=O)C(C#N)=C[C@@]2(CCC)c3nc(-c4ccnc(C5CC5)c4)nc(-c4ccccc4F)c3CC[C@H]12 ; CCC[C@@]12C=C(C#N)C(=O)[C@H](C)[C@H]1CCc1c(-c3ccccc3F)nc(-c3ccnc(C4CC4)c3)nc12 ; CC(C)CC[C@@]12C=C(C#N)C(=O)C[C@H]1CCc1c(-c3ccccc3F)nc(-c3ccnc(CF)c3)nc12 ; CC(C)CC[C@@]12C=C(C#N)C(=O)C[C@H]1CCc1c(-c3ccccc3F)nc(-c3ccnc4ccccc34)nc12]

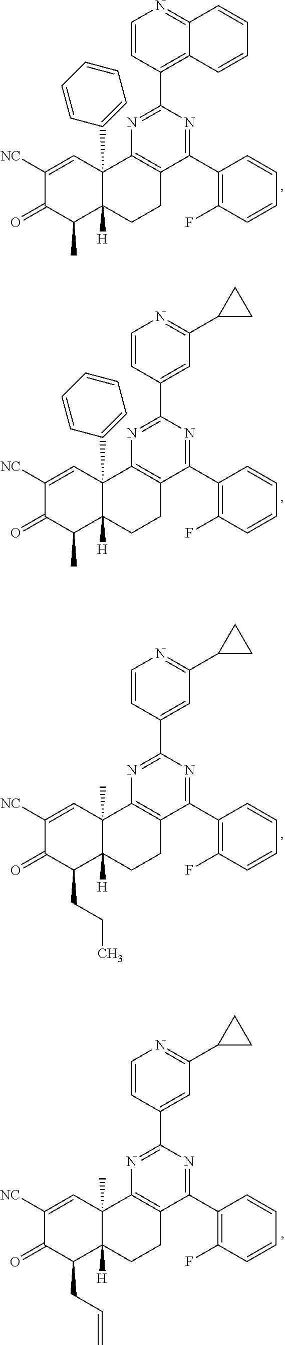 [SMILES: C[C@H]1C(=O)C(C#N)=C[C@@]2(c3ccccc3)c3nc(-c4ccnc5ccccc45)nc(-c4ccccc4F)c3CC[C@H]12 ; C[C@H]1C(=O)C(C#N)=C[C@@]2(c3ccccc3)c3nc(-c4ccnc(C5CC5)c4)nc(-c4ccccc4F)c3CC[C@H]12 ; CCC[C@H]1C(=O)C(C#N)=C[C@@]2(C)c3nc(-c4ccnc(C5CC5)c4)nc(-c4ccccc4F)c3CC[C@H]12 ; C=CC[C@H]1C(=O)C(C#N)=C[C@@]2(C)c3nc(-c4ccnc(C5CC5)c4)nc(-c4ccccc4F)c3CC[C@H]12]

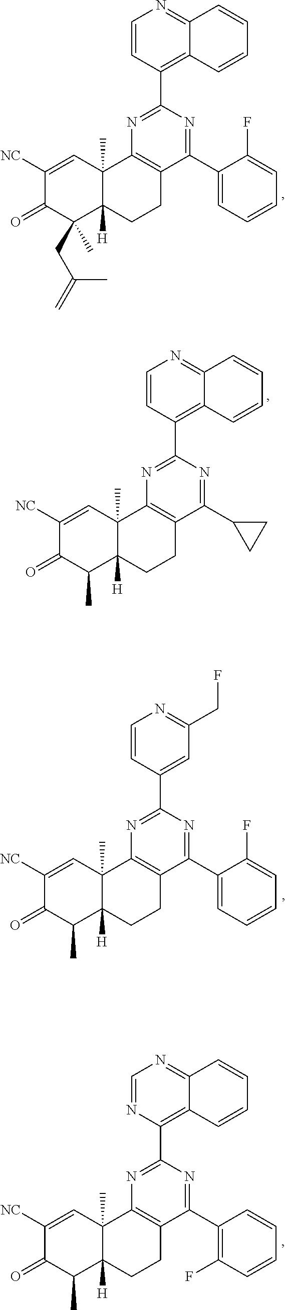 [SMILES: C=C(C)C[C@@]1(C)C(=O)C(C#N)=C[C@@]2(C)c3nc(-c4ccnc5ccccc45)nc(-c4ccccc4F)c3CC[C@H]12 ; C[C@H]1C(=O)C(C#N)=C[C@@]2(C)c3nc(-c4ccnc5ccccc45)nc(C4CC4)c3CC[C@H]12 ; C[C@H]1C(=O)C(C#N)=C[C@@]2(C)c3nc(-c4ccnc(CF)c4)nc(-c4ccccc4F)c3CC[C@H]12 ; C[C@H]1C(=O)C(C#N)=C[C@@]2(C)c3nc(-c4ncnc5ccccc45)nc(-c4ccccc4F)c3CC[C@H]12]

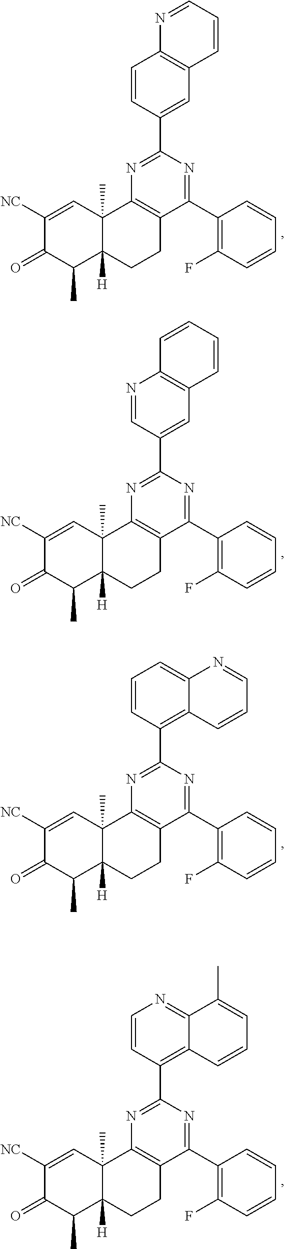 [SMILES: C[C@H]1C(=O)C(C#N)=C[C@@]2(C)c3nc(-c4ccc5ncccc5c4)nc(-c4ccccc4F)c3CC[C@H]12 ; C[C@H]1C(=O)C(C#N)=C[C@@]2(C)c3nc(-c4cnc5ccccc5c4)nc(-c4ccccc4F)c3CC[C@H]12 ; C[C@H]1C(=O)C(C#N)=C[C@@]2(C)c3nc(-c4cccc5ncccc45)nc(-c4ccccc4F)c3CC[C@H]12 ; Cc1cccc2c(-c3nc(-c4ccccc4F)c4c(n3)[C@]3(C)C=C(C#N)C(=O)[C@H](C)[C@H]3CC4)ccnc12]

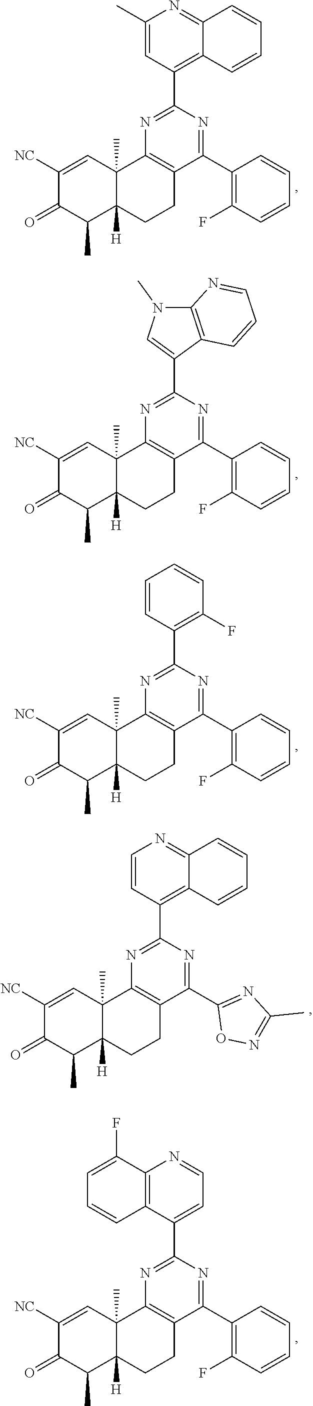 [SMILES: Cc1cc(-c2nc(-c3ccccc3F)c3c(n2)[C@]2(C)C=C(C#N)C(=O)[C@H](C)[C@H]2CC3)c2ccccc2n1 ; C[C@H]1C(=O)C(C#N)=C[C@@]2(C)c3nc(-c4cn(C)c5ncccc45)nc(-c4ccccc4F)c3CC[C@H]12 ; C[C@H]1C(=O)C(C#N)=C[C@@]2(C)c3nc(-c4ccccc4F)nc(-c4ccccc4F)c3CC[C@H]12 ; Cc1noc(-c2nc(-c3ccnc4ccccc34)nc3c2CC[C@@H]2[C@@H](C)C(=O)C(C#N)=C[C@@]32C)n1 ; C[C@H]1C(=O)C(C#N)=C[C@@]2(C)c3nc(-c4ccnc5c(F)cccc45)nc(-c4ccccc4F)c3CC[C@H]12]

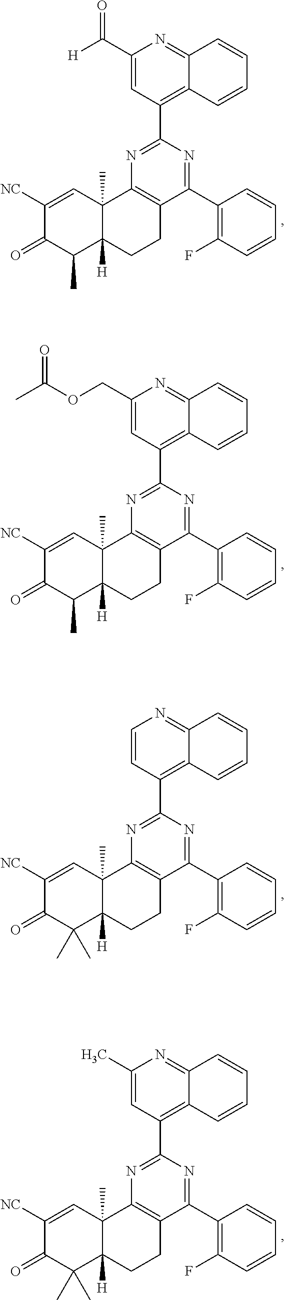 [SMILES: C[C@H]1C(=O)C(C#N)=C[C@@]2(C)c3nc(-c4cc(C=O)nc5ccccc45)nc(-c4ccccc4F)c3CC[C@H]12 ; CC(=O)OCc1cc(-c2nc(-c3ccccc3F)c3c(n2)[C@]2(C)C=C(C#N)C(=O)[C@H](C)[C@H]2CC3)c2ccccc2n1 ; CC1(C)C(=O)C(C#N)=C[C@@]2(C)c3nc(-c4ccnc5ccccc45)nc(-c4ccccc4F)c3CC[C@H]12 ; Cc1cc(-c2nc(-c3ccccc3F)c3c(n2)[C@]2(C)C=C(C#N)C(=O)C(C)(C)[C@H]2CC3)c2ccccc2n1]

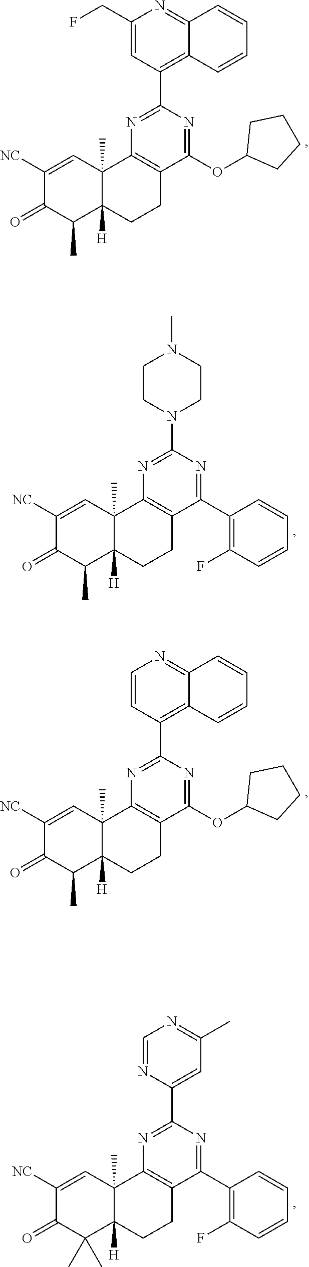 [SMILES: C[C@H]1C(=O)C(C#N)=C[C@@]2(C)c3nc(-c4cc(CF)nc5ccccc45)nc(OC4CCCC4)c3CC[C@H]12 ; C[C@H]1C(=O)C(C#N)=C[C@@]2(C)c3nc(N4CCN(C)CC4)nc(-c4ccccc4F)c3CC[C@H]12 ; C[C@H]1C(=O)C(C#N)=C[C@@]2(C)c3nc(-c4ccnc5ccccc45)nc(OC4CCCC4)c3CC[C@H]12 ; Cc1cc(-c2nc(-c3ccccc3F)c3c(n2)[C@]2(C)C=C(C#N)C(=O)C(C)(C)[C@H]2CC3)ncn1]

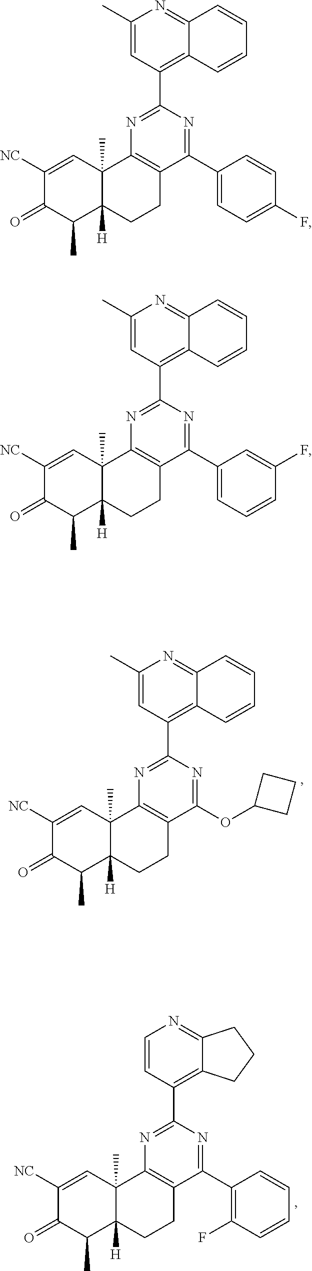 [SMILES: Cc1cc(-c2nc(-c3ccc(F)cc3)c3c(n2)[C@]2(C)C=C(C#N)C(=O)[C@H](C)[C@H]2CC3)c2ccccc2n1 ; Cc1cc(-c2nc(-c3cccc(F)c3)c3c(n2)[C@]2(C)C=C(C#N)C(=O)[C@H](C)[C@H]2CC3)c2ccccc2n1 ; Cc1cc(-c2nc(OC3CCC3)c3c(n2)[C@]2(C)C=C(C#N)C(=O)[C@H](C)[C@H]2CC3)c2ccccc2n1 ; C[C@H]1C(=O)C(C#N)=C[C@@]2(C)c3nc(-c4ccnc5c4CCC5)nc(-c4ccccc4F)c3CC[C@H]12]

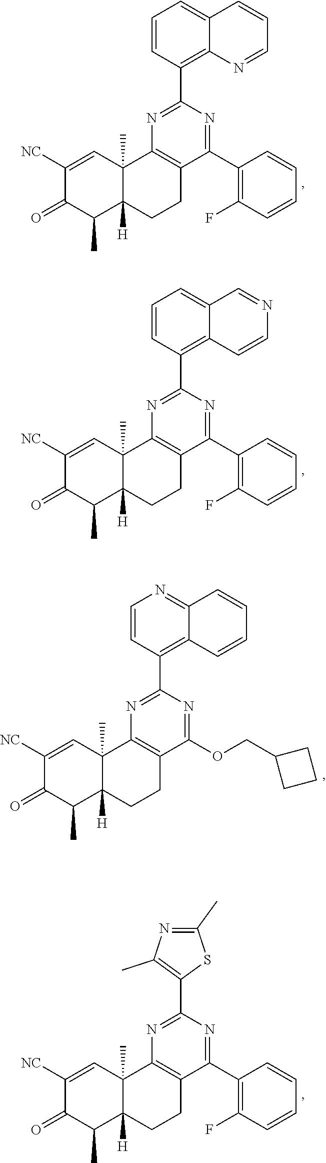 [SMILES: C[C@H]1C(=O)C(C#N)=C[C@@]2(C)c3nc(-c4cccc5cccnc45)nc(-c4ccccc4F)c3CC[C@H]12 ; C[C@H]1C(=O)C(C#N)=C[C@@]2(C)c3nc(-c4cccc5cnccc45)nc(-c4ccccc4F)c3CC[C@H]12 ; C[C@H]1C(=O)C(C#N)=C[C@@]2(C)c3nc(-c4ccnc5ccccc45)nc(OCC4CCC4)c3CC[C@H]12 ; Cc1nc(C)c(-c2nc(-c3ccccc3F)c3c(n2)[C@]2(C)C=C(C#N)C(=O)[C@H](C)[C@H]2CC3)s1]

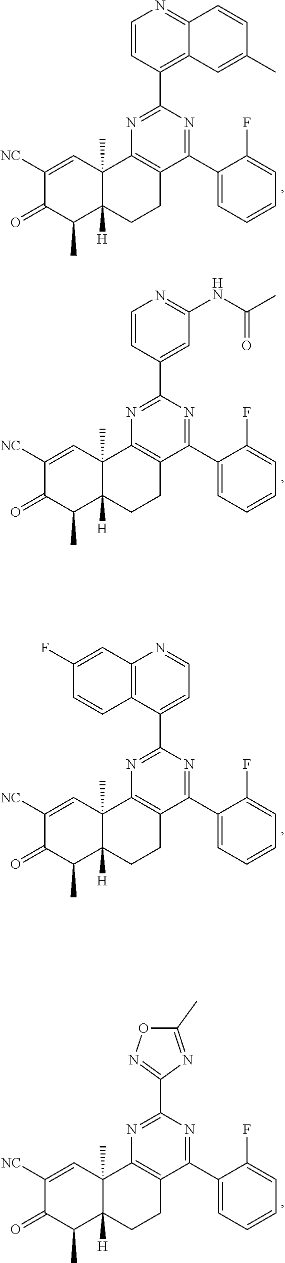 [SMILES: Cc1ccc2nccc(-c3nc(-c4ccccc4F)c4c(n3)[C@]3(C)C=C(C#N)C(=O)[C@H](C)[C@H]3CC4)c2c1 ; CC(=O)Nc1cc(-c2nc(-c3ccccc3F)c3c(n2)[C@]2(C)C=C(C#N)C(=O)[C@H](C)[C@H]2CC3)ccn1 ; C[C@H]1C(=O)C(C#N)=C[C@@]2(C)c3nc(-c4ccnc5cc(F)ccc45)nc(-c4ccccc4F)c3CC[C@H]12 ; Cc1nc(-c2nc(-c3ccccc3F)c3c(n2)[C@]2(C)C=C(C#N)C(=O)[C@H](C)[C@H]2CC3)no1]

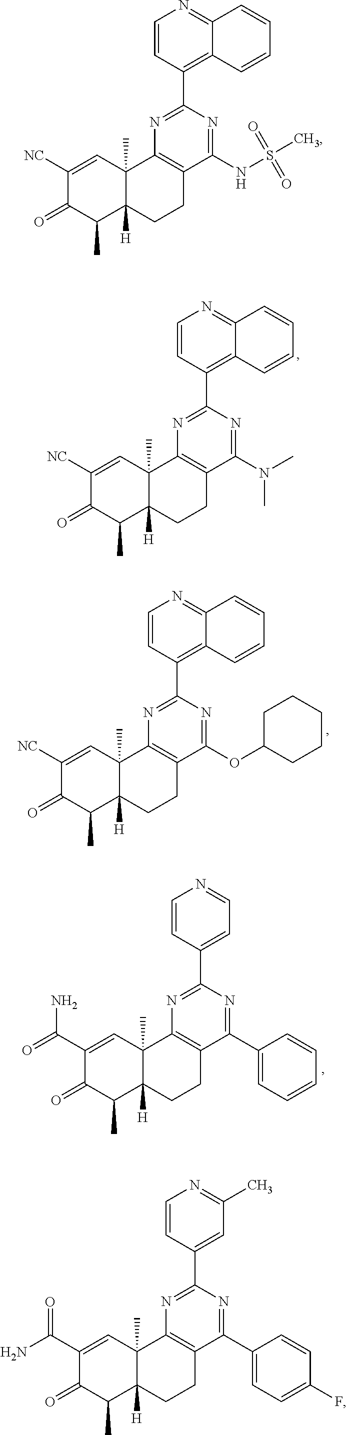 [SMILES: C[C@H]1C(=O)C(C#N)=C[C@@]2(C)c3nc(-c4ccnc5ccccc45)nc(NS(C)(=O)=O)c3CC[C@H]12 ; C[C@H]1C(=O)C(C#N)=C[C@@]2(C)c3nc(-c4ccnc5ccccc45)nc(N(C)C)c3CC[C@H]12 ; C[C@H]1C(=O)C(C#N)=C[C@@]2(C)c3nc(-c4ccnc5ccccc45)nc(OC4CCCCC4)c3CC[C@H]12 ; C[C@H]1C(=O)C(C(N)=O)=C[C@@]2(C)c3nc(-c4ccncc4)nc(-c4ccccc4)c3CC[C@H]12 ; Cc1cc(-c2nc(-c3ccc(F)cc3)c3c(n2)[C@]2(C)C=C(C(N)=O)C(=O)[C@H](C)[C@H]2CC3)ccn1]

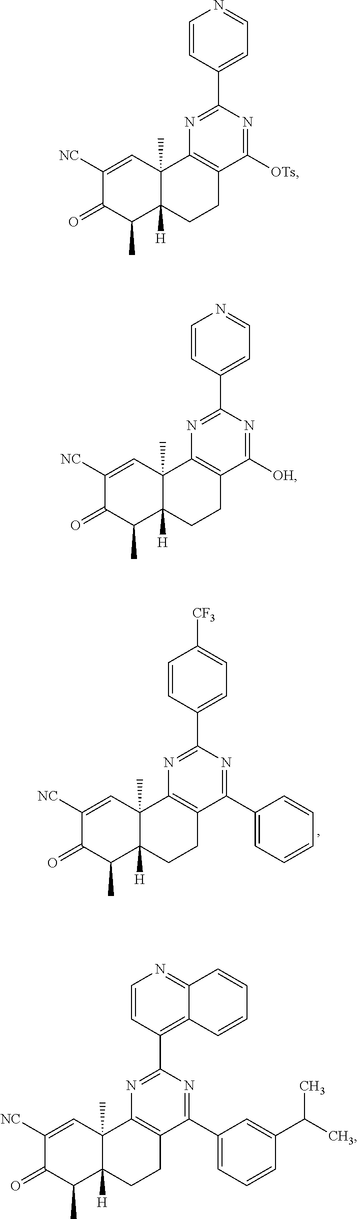 [SMILES: Cc1ccc(S(=O)(=O)Oc2nc(-c3ccncc3)nc3c2CC[C@@H]2[C@@H](C)C(=O)C(C#N)=C[C@@]32C)cc1 ; C[C@H]1C(=O)C(C#N)=C[C@@]2(C)c3nc(-c4ccncc4)nc(O)c3CC[C@H]12 ; C[C@H]1C(=O)C(C#N)=C[C@@]2(C)c3nc(-c4ccc(C(F)(F)F)cc4)nc(-c4ccccc4)c3CC[C@H]12 ; CC(C)c1cccc(-c2nc(-c3ccnc4ccccc34)nc3c2CC[C@@H]2[C@@H](C)C(=O)C(C#N)=C[C@@]32C)c1]

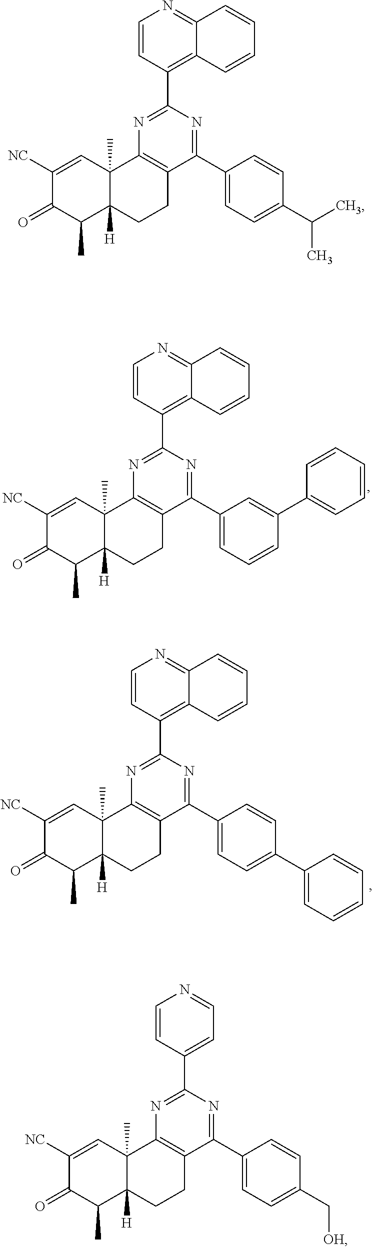 [SMILES: CC(C)c1ccc(-c2nc(-c3ccnc4ccccc34)nc3c2CC[C@@H]2[C@@H](C)C(=O)C(C#N)=C[C@@]32C)cc1 ; C[C@H]1C(=O)C(C#N)=C[C@@]2(C)c3nc(-c4ccnc5ccccc45)nc(-c4cccc(-c5ccccc5)c4)c3CC[C@H]12 ; C[C@H]1C(=O)C(C#N)=C[C@@]2(C)c3nc(-c4ccnc5ccccc45)nc(-c4ccc(-c5ccccc5)cc4)c3CC[C@H]12 ; C[C@H]1C(=O)C(C#N)=C[C@@]2(C)c3nc(-c4ccncc4)nc(-c4ccc(CO)cc4)c3CC[C@H]12]

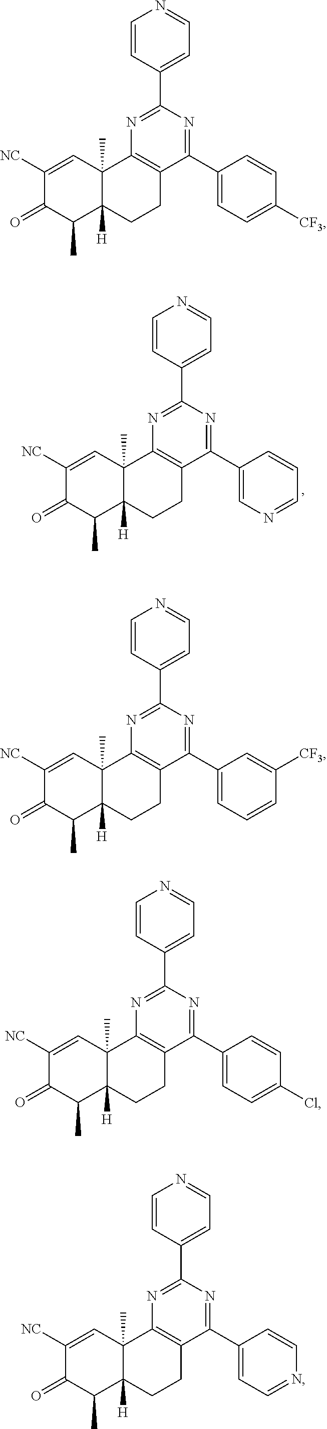 [SMILES: C[C@H]1C(=O)C(C#N)=C[C@@]2(C)c3nc(-c4ccncc4)nc(-c4ccc(C(F)(F)F)cc4)c3CC[C@H]12 ; C[C@H]1C(=O)C(C#N)=C[C@@]2(C)c3nc(-c4ccncc4)nc(-c4cccnc4)c3CC[C@H]12 ; C[C@H]1C(=O)C(C#N)=C[C@@]2(C)c3nc(-c4ccncc4)nc(-c4cccc(C(F)(F)F)c4)c3CC[C@H]12 ; C[C@H]1C(=O)C(C#N)=C[C@@]2(C)c3nc(-c4ccncc4)nc(-c4ccc(Cl)cc4)c3CC[C@H]12 ; C[C@H]1C(=O)C(C#N)=C[C@@]2(C)c3nc(-c4ccncc4)nc(-c4ccncc4)c3CC[C@H]12]

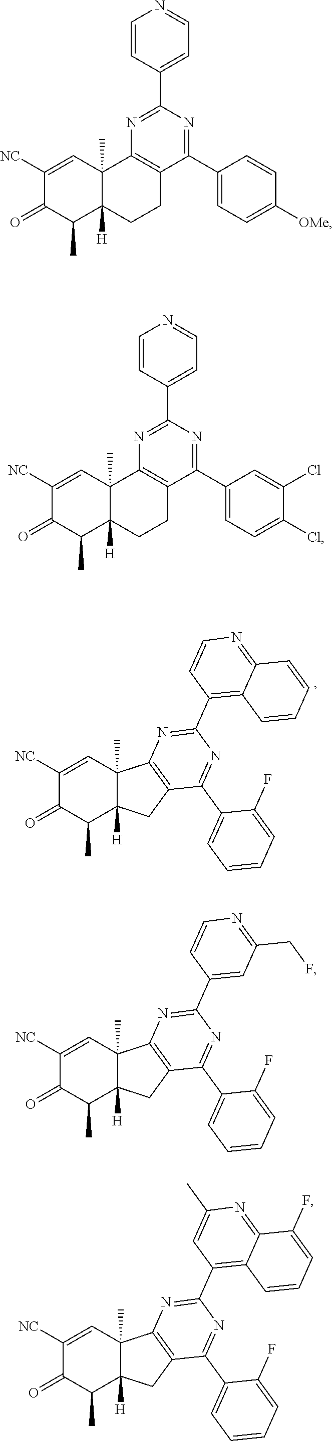 [SMILES: COc1ccc(-c2nc(-c3ccncc3)nc3c2CC[C@@H]2[C@@H](C)C(=O)C(C#N)=C[C@@]32C)cc1 ; C[C@H]1C(=O)C(C#N)=C[C@@]2(C)c3nc(-c4ccncc4)nc(-c4ccc(Cl)c(Cl)c4)c3CC[C@H]12 ; C[C@H]1C(=O)C(C#N)=C[C@@]2(C)c3nc(-c4ccnc5ccccc45)nc(-c4ccccc4F)c3C[C@H]12 ; C[C@H]1C(=O)C(C#N)=C[C@@]2(C)c3nc(-c4ccnc(CF)c4)nc(-c4ccccc4F)c3C[C@H]12 ; Cc1cc(-c2nc(-c3ccccc3F)c3c(n2)[C@]2(C)C=C(C#N)C(=O)[C@H](C)[C@H]2C3)c2cccc(F)c2n1]

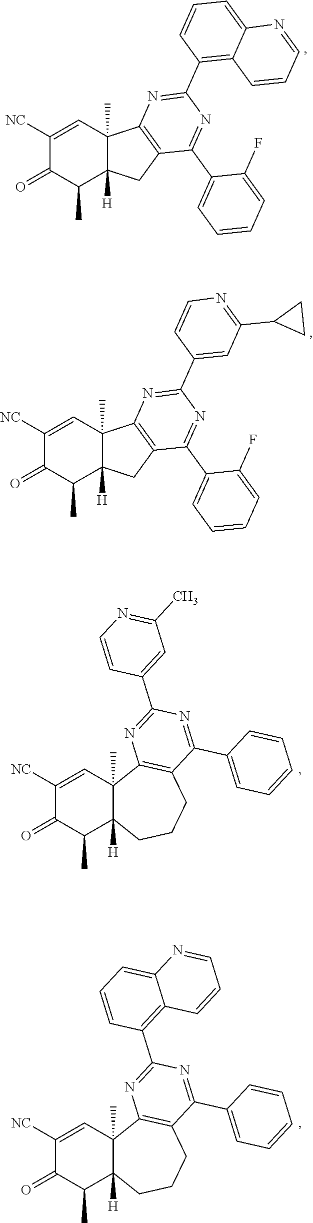 [SMILES: C[C@H]1C(=O)C(C#N)=C[C@@]2(C)c3nc(-c4cccc5ncccc45)nc(-c4ccccc4F)c3C[C@H]12 ; C[C@H]1C(=O)C(C#N)=C[C@@]2(C)c3nc(-c4ccnc(C5CC5)c4)nc(-c4ccccc4F)c3C[C@H]12 ; Cc1cc(-c2nc(-c3ccccc3)c3c(n2)[C@]2(C)C=C(C#N)C(=O)[C@H](C)[C@H]2CCC3)ccn1 ; C[C@H]1C(=O)C(C#N)=C[C@@]2(C)c3nc(-c4cccc5ncccc45)nc(-c4ccccc4)c3CCC[C@H]12]

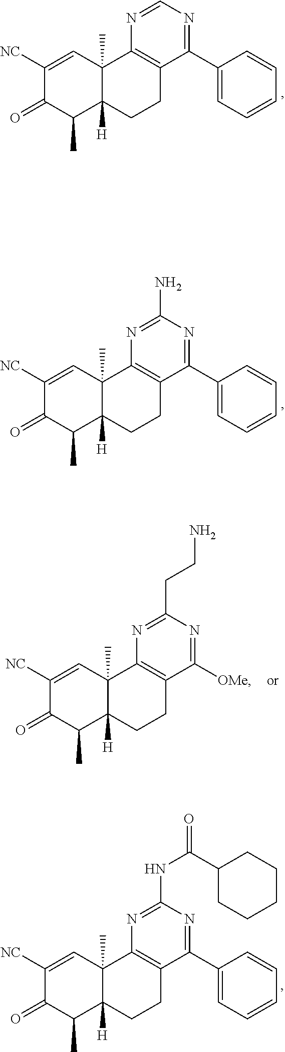 [SMILES: C[C@H]1C(=O)C(C#N)=C[C@@]2(C)c3ncnc(-c4ccccc4)c3CC[C@H]12 ; C[C@H]1C(=O)C(C#N)=C[C@@]2(C)c3nc(N)nc(-c4ccccc4)c3CC[C@H]12 ; COc1nc(CCN)nc2c1CC[C@@H]1[C@@H](C)C(=O)C(C#N)=C[C@@]21C ; C[C@H]1C(=O)C(C#N)=C[C@@]2(C)c3nc(NC(=O)C4CCCCC4)nc(-c4ccccc4)c3CC[C@H]12]

or a pharmaceutically acceptable salt thereof.

In some embodiments, the inverse agonist is a compound of the formula:

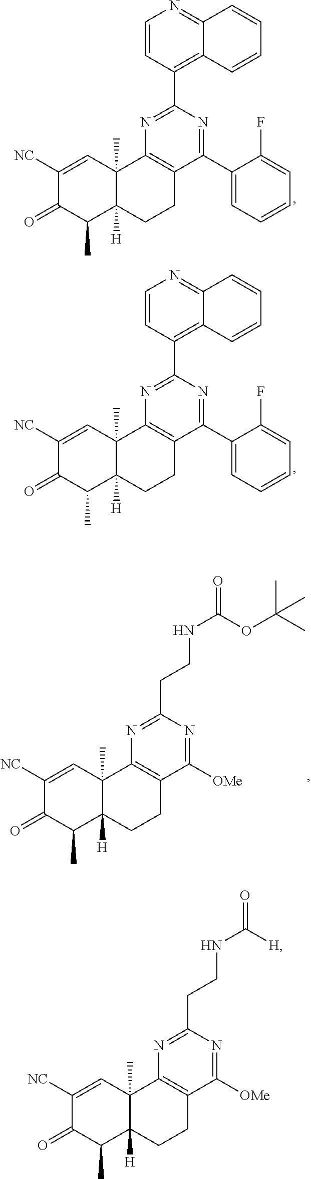

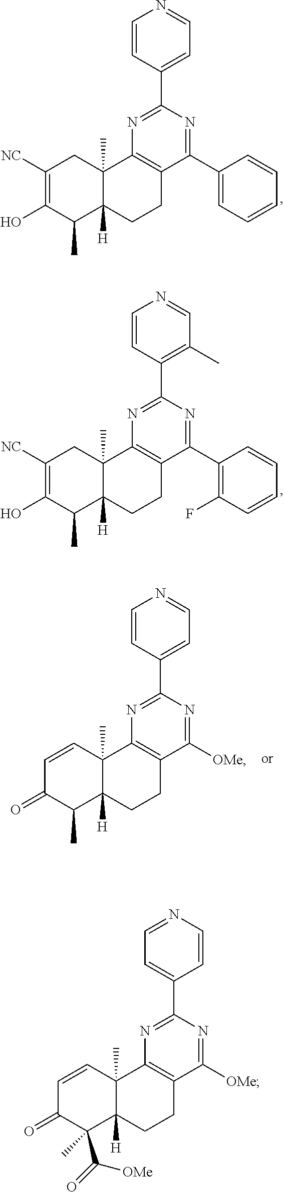 [SMILES: C[C@H]1C(O)=C(C#N)C[C@@]2(C)c3nc(-c4ccncc4)nc(-c4ccccc4)c3CC[C@H]12 ; Cc1cnccc1-c1nc(-c2ccccc2F)c2c(n1)[C@]1(C)CC(C#N)=C(O)[C@H](C)[C@H]1CC2 ; COc1nc(-c2ccncc2)nc2c1CC[C@@H]1[C@@H](C)C(=O)C=C[C@@]21C ; "or" ; COC(=O)[C@@]1(C)C(=O)C=C[C@@]2(C)c3nc(-c4ccncc4)nc(OC)c3CC[C@H]12]

or a pharmaceutically acceptable salt thereof.

In some embodiments, the inverse agonist is a compound of the formula:

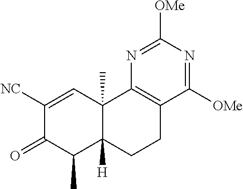 [SMILES: COc1nc(OC)c2c(n1)[C@]1(C)C=C(C#N)C(=O)[C@H](C)[C@H]1CC2]

or a pharmaceutically acceptable salt thereof.

In some embodiments, the inverse agonist is a compound of the formula:

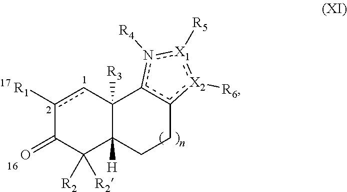

wherein:
wherein the bond between carbon atoms 1 and 2 is an epoxidized double bond or a double bond;
n is 0, 1, or 2;
R¹ is cyano, fluoro, —CF₃, or —C(O)Rₐ, wherein:
   Rₐ is hydroxy or amino; or
      alkoxy_{(C≤6)}, alkylamino_{(C≤6)}, dialkylamino_{(C≤6)}, or a substituted version of any of these groups;
R₂ and R₂′ are each independently hydrogen; or
   alkyl_{(C≤12)}, cycloalkyl_{(C≤12)}, alkenyl_{(C≤12)}, alkynyl_{(C≤12)}, aryl_{(C≤18)}, aralkyl_{(C≤18)}, heteroaryl_{(C≤18)}, heteroaralkyl_{(C≤18)}, or a substituted version of any of these groups; or
   R₂ and R₂′ are taken together and are alkanediyl_{(C≤8)}, alkenediyl_{(C≤8)}, or a substituted version of either of these groups;
R₃ is alkyl_{(C≤12)}, alkenyl_{(C≤12)}, aryl_{(C≤12)}, aralkyl_{(C≤12)}, or a substituted version of any of these groups;
R₄ and R₅ are each independently absent or hydrogen; or
   alkyl_{(C≤12)}, cycloalkyl_{(C≤12)}, heterocycloalkyl_{(C≤12)}, aryl_{(C≤12)}, aralkyl_{(C≤12)}, heteroaryl_{(C≤12)}, heteroaralkyl_{(C≤12)}, -arenediyl_{(C≤12)}-alkyl_{(C≤12)}, -arenediyl_{(C≤12)}-aryl_{(C≤12)}, -arenediyl_{(C≤12)}-heteroaryl_{(C≤12)}, -arenediyl_{(C≤12)}-heterocycloalkyl_{(C≤12)}, -arenediyl_{(C≤12)}-cycloalkyl_{(C≤12)}, -heteroarenediyl_{(C≤12)}-alkyl_{(C≤12)}, -heteroarenediyl_{(C≤12)}-aryl_{(C≤12)}, -hetero-arenediyl_{(C≤12)}-heteroaryl_{(C≤12)}, -heteroarenediyl_{(C≤12)}-heterocycloalkyl_{(C≤12)}, -heteroarenediyl_{(C≤12)}-cycloalkyl_{(C≤12)}, -heterocyclo-alkanediyl_{(C≤12)}-aryl_{(C≤12)}, -heterocycloalkanediyl_{(C≤12)}-heteroaryl_{(C≤12)}, or a substituted version of any of these groups; or
   a group of the formula:

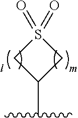

wherein 1 and m are each 0, 1, 2, or 3;
R₆ is absent, hydrogen, or amino; or
   alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, diarylamino_{(C≤12)}, alkyl_{(C≤12)}, cycloalkyl_{(C≤12)}, -alkanediyl_{(C≤12)}-cycloalkyl_{(C≤12)}, -alkanediyl_{(C≤18)}-aralkoxy_{(C≤18)}, heterocycloalkyl_{(C≤12)}, aryl_{(C≤18)}, -arenediyl_{(C≤12)}-alkyl_{(C≤12)}, aralkyl_{(C≤18)}, -arenediyl_{(C≤8)}-heterocycloalkyl_{(C≤12)}, heteroaryl_{(C≤18)}, -heteroarenediyl_{(C≤12)}-alkyl_{(C≤12)}, heteroaralkyl_{(C≤18)}, acyl_{(C≤12)}, alkoxy_{(C≤12)}, or a substituted version of any of these groups; and
   X₁ and X₂ are each independently C or N, provided that X₂ is C when R₆ is amino, alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, or diarylamino_{(C≤12)};
      or a pharmaceutically acceptable salt thereof.

In some embodiments, the compound is further defined as:

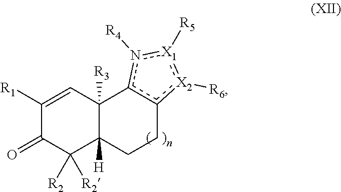

wherein:
n is 0, 1, or 2;
R¹ is cyano, fluoro, —CF₃, or —C(O)Rₐ, wherein
   Rₐ is hydroxy or amino; or
      alkoxy_{(C≤6)}, alkylamino_{(C≤6)}, dialkylamino_{(C≤6)}, or a substituted version of any of these groups;
R₂ and R₂′ are each independently hydrogen; or
   alkyl_{(C≤12)}, cycloalkyl_{(C≤12)}, alkenyl_{(C≤12)}, alkynyl_{(C≤12)}, aryl_{(C≤18)}, aralkyl_{(C≤18)}, heteroaryl_{(C≤12)}, heteroaralkyl_{(C≤8)}, or a substituted version of any of these groups; or
   R₂ and R₂′ are taken together and are alkanediyl_{(C≤8)}, alkenediyl_{(C≤8)}, or a substituted version of either of these groups;
R₃ is alkyl_{(C≤12)}, alkenyl_{(C≤12)}, aryl_{(C≤12)}, aralkyl_{(C≤12)}, or a substituted version of any of these groups;
R₄ and R₅ are each independently absent or hydrogen; or
   alkyl_{(C≤12)}, cycloalkyl_{(C≤12)}, heterocycloalkyl_{(C≤12)}, aryl_{(C≤12)}, aralkyl_{(C≤12)}, heteroaryl_{(C≤12)}, heteroaralkyl_{(C≤12)}, -arenediyl_{(C≤12)}-alkyl_{(C≤12)}, -arenediyl_{(C≤12)}-aryl_{(C≤12)}, -arenediyl_{(C≤12)}-heteroaryl_{(C≤12)}, -arenediyl_{(C≤12)}-heterocycloalkyl_{(C≤12)}, -arenediyl_{(C≤12)}-cycloalkyl_{(C≤12)}, -heteroarenediyl_{(C≤12)}-alkyl_{(C≤12)}, -heteroarenediyl_{(C≤12)}-aryl_{(C≤12)}, -hetero-arenediyl_{(C≤12)}-heteroaryl_{(C≤12)}, -heteroarenediyl_{(C≤12)}-heterocycloalkyl_{(C≤12)}, -heteroarenediyl_{(C≤12)}-cycloalkyl_{(C≤12)}, -heterocyclo-alkanediyl_{(C≤12)}-aryl_{(C≤12)}, -heterocycloalkanediyl_{(C≤12)}-heteroaryl_{(C≤12)}, or a substituted version of any of these groups; or
   a group of the formula:

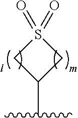

wherein 1 and m are each 0, 1, 2, or 3;
R₆ is absent, hydrogen, or amino; or
   alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, diarylamino_{(C≤12)}, alkyl_{(C≤12)}, cycloalkyl_{(C≤12)}, -alkanediyl_{(C≤12)}-cycloalkyl_{(C≤12)}, -alkanediyl_{(C≤18)}-aralkoxy_{(C≤18)}, heterocycloalkyl_{(C≤12)}, aryl_{(C≤18)}, -arenediyl_{(C≤12)}-alkyl_{(C≤12)}, aralkyl_{(C≤18)}, -arenediyl_{(C≤18)}-heterocycloalkyl_{(C≤12)}, heteroaryl_{(C≤8)}, -heteroarenediyl_{(C≤12)}-alkyl_{(C≤12)}, heteroaralkyl_{(C≤18)}, acyl_{(C≤12)}, alkoxy_{(C≤12)}, or a substituted version of any of these groups; and
X₁ and X₂ are each independently C or N, provided that X₂ is C when R₆ is amino, alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, or diarylamino_{(C≤12)};
   or a pharmaceutically acceptable salt thereof.

In some embodiments, the compound is further defined as:

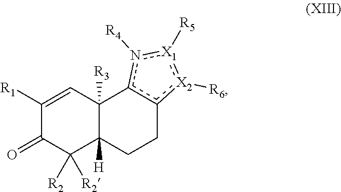

wherein:
R¹ is cyano, fluoro, —CF₃, or —C(O)Rₐ, wherein
   Rₐ is hydroxy or amino; or
      alkoxy_{(C≤6)}, alkylamino_{(C≤6)}, dialkylamino_{(C≤6)}, or a substituted version of any of these groups;
R₂ and R₂′ are each independently hydrogen; or
   alkyl_{(C≤12)}, cycloalkyl_{(C≤12)}, alkenyl_{(C≤12)}, alkynyl_{(C≤12)}, aryl_{(C≤8)}, aralkyl_{(C≤8)}, heteroaryl_{(C≤8)}, heteroaralkyl_{(C≤8)}, or a substituted version of any of these groups; or
   R₂ and R₂′ are taken together and are alkanediyl_{(C≤8)}, alkenediyl_{(C≤8)}, or a substituted version of either of these groups;
R₃ is alkyl_{(C≤12)}, alkenyl_{(C≤12)}, aryl_{(C≤12)}, aralkyl_{(C≤12)}, or a substituted version of any of these groups;
R₄ and R₅ are each independently absent or hydrogen; or
   alkyl_{(C≤12)}, cycloalkyl_{(C≤12)}, heterocycloalkyl_{(C≤12)}, aryl_{(C≤12)}, aralkyl_{(C≤12)}, heteroaryl_{(C≤12)}, heteroaralkyl_{(C≤12)}, -arenediyl_{(C≤12)}-alkyl_{(C≤12)}, -arenediyl_{(C≤12)}-aryl_{(C≤12)}, -arenediyl_{(C≤12)}-heteroaryl_{(C≤12)}, -arenediyl_{(C≤12)}-heterocycloalkyl_{(C≤12)}, -arenediyl_{(C≤12)}-cycloalkyl_{(C≤12)}, -heteroarenediyl_{(C≤12)}-alkyl_{(C≤12)}, -heteroarenediyl_{(C≤12)}-aryl_{(C≤12)}, -hetero-arenediyl_{(C≤12)}-heteroaryl_{(C≤12)}, -heteroarenediyl_{(C≤12)}-heterocycloalkyl_{(C≤12)}, -heteroarenediyl_{(C≤12)}-cycloalkyl_{(C≤12)}, -heterocyclo-alkanediyl_{(C≤12)}-aryl_{(C≤12)}, -heterocycloalkanediyl_{(C≤12)}-heteroaryl_{(C≤12)}, or a substituted version of any of these groups; or
   a group of the formula:

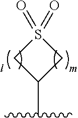

wherein 1 and m are each 0, 1, 2, or 3;
R₆ is absent, hydrogen, or amino; or
   alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, diarylamino_{(C≤12)}, alkyl_{(C≤12)}, cycloalkyl_{(C≤12)}, -alkanediyl_{(C≤12)}-cycloalkyl_{(C≤12)}, -alkanediyl_{(C≤18)}-aralkoxy_{(C≤18)}, heterocycloalkyl_{(C≤12)}, aryl_{(C≤18)}, -arenediyl_{(C≤12)}-alkyl_{(C≤12)}, aralkyl_{(C≤18)}, -arenediyl_{(C≤8)}-heterocycloalkyl_{(C≤12)}, heteroaryl_{(C≤18)}, -heteroarenediyl_{(C≤12)}-alkyl_{(C≤12)}, heteroaralkyl_{(C≤8)}, acyl_{(C≤12)}, alkoxy_{(C≤12)}, or a substituted version of any of these groups; and
X₁ and X₂ are each independently C or N, provided that X₂ is C when R₆ is amino, alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, or diarylamino_{(C≤12)};
   or a pharmaceutically acceptable salt thereof.

In some embodiments, the compound is further defined as:

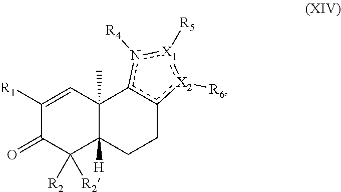

wherein:
R₁ is cyano, fluoro, —CF₃, or —C(O)Rₐ, wherein
   Rₐ is hydroxy or amino; or
      alkoxy_{(C≤6)}, alkylamino_{(C≤6)}, dialkylamino_{(C≤6)}, or a substituted version of any of these groups;
R₂ and R₂′ are each independently hydrogen; or
   alkyl_{(C≤12)}, cycloalkyl_{(C≤12)}, alkenyl_{(C≤12)}, alkynyl_{(C≤12)}, aryl_{(C≤18)}, aralkyl_{(C≤18)}, heteroaryl_{(C≤18)}, heteroaralkyl_{(C≤18)}, or a substituted version of any of these groups; or
   R² and R₂′ are taken together and are alkanediyl_{(C≤8)}, alkenediyl_{(C≤8)}, or a substituted version of either of these groups;
R₄ and R₅ are each independently absent or hydrogen; or
   alkyl_{(C≤12)}, cycloalkyl_{(C≤12)}, heterocycloalkyl_{(C≤12)}, aryl_{(C≤12)}, aralkyl_{(C≤12)}, heteroaryl_{(C≤12)}, heteroaralkyl_{(C≤12)}, -arenediyl_{(C≤12)}-alkyl_{(C≤12)}, -arenediyl_{(C≤12)}-aryl_{(C≤12)}, -arenediyl_{(C≤12)}-heteroaryl_{(C≤12)}, -arenediyl_{(C≤18)}-heterocycloalkyl_{(C≤12)}, -arenediyl_{(C≤12)}-cycloalkyl_{(C≤12)}, -heteroarenediyl_{(C≤12)}-alkyl_{(C≤12)}, -heteroarenediyl_{(C≤12)}-aryl_{(C≤12)}, -heteroarenediyl_{(C≤12)}-heteroaryl_{(C≤12)}, -heteroarenediyl_{(C≤12)}-hetero-cycloalkyl_{(C≤12)}, -heteroarenediyl_{(C≤12)}-cycloalkyl_{(C≤12)}, -heterocycloalkanediyl_{(C≤12)}-aryl_{(C≤12)}, -heterocycloalkanediyl_{(C≤12)}-heteroaryl_{(C≤12)}, or a substituted version of any of these groups; or
   a group of the formula:

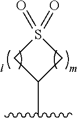

wherein 1 and m are each 0, 1, 2, or 3;
R₆ is absent, hydrogen, or amino; or
   alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, diarylamino_{(C≤12)}, alkyl_{(C≤12)}, cycloalkyl_{(C≤12)}, -alkanediyl_{(C≤12)}-cycloalkyl_{(C≤12)}, -alkanediyl_{(C≤18)}-aralkoxy_{(C≤18)}, heterocycloalkyl_{(C≤12)}, aryl_{(C≤18)}, -arenediyl_{(C≤12)}-alkyl_{(C≤12)}, aralkyl_{(C≤8)}, -arenediyl_{(C≤18)}-heterocycloalkyl_{(C≤12)}, heteroaryl_{(C≤8)}, -heteroarenediyl_{(C≤12)}-alkyl_{(C≤12)}, heteroaralkyl_{(C≤18)}, acyl_{(C≤12)}, alkoxy_{(C≤12)}, or a substituted version of any of these groups; and
X₁ and X₂ are each independently C or N, provided that X₂ is C when R₆ is amino, alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, or diarylamino_{(C≤12)};
   or a pharmaceutically acceptable salt thereof.

In some embodiments, the compound is further defined as:

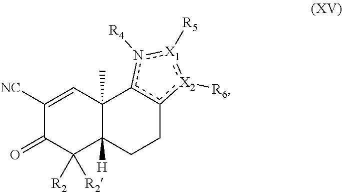

wherein:
R₂ and R₂′ are each independently hydrogen; or
   alkyl_{(C≤12)}, cycloalkyl_{(C≤12)}, alkenyl_{(C≤12)}, alkynyl_{(C≤12)}, aryl_{(C≤8)}, aralkyl_{(C≤18)}, heteroaryl_{(C≤18)}, heteroaralkyl_{(C≤18)}, or a substituted version of any of these groups; or
   R₂ and R₂′ are taken together and are alkanediyl_{(C≤8)}, alkenediyl_{(C≤8)}, or a substituted version of either of these groups;
R₄ and R₅ are each independently absent or hydrogen; or
   alkyl_{(C≤12)}, cycloalkyl_{(C≤12)}, heterocycloalkyl_{(C≤12)}, aryl_{(C≤12)}, aralkyl_{(C≤12)}, heteroaryl_{(C≤12)}, heteroaralkyl_{(C≤12)}, -arenediyl_{(C≤12)}-alkyl_{(C≤12)}, -arenediyl_{(C≤12)}-aryl_{(C≤12)}, -arenediyl_{(C≤12)}-heteroaryl_{(C≤12)}, -arenediyl_{(C≤12)}-heterocycloalkyl_{(C≤12)}, -arenediyl_{(C≤12)}-cycloalkyl_{(C≤12)}, -heteroarenediyl_{(C≤12)}-alkyl_{(C≤12)}, -heteroarenediyl_{(C≤12)}-aryl_{(C≤12)}, -hetero-arenediyl_{(C≤12)}-heteroaryl_{(C≤12)}, -heteroarenediyl_{(C≤12)}-heterocycloalkyl_{(C≤12)}, -heteroarenediyl_{(C≤12)}-cycloalkyl_{(C≤12)}, -heterocyclo-alkanediyl_{(C≤12)}-aryl_{(C≤12)}, -heterocycloalkanediyl_{(C≤12)}-heteroaryl_{(C≤12)}, or a substituted version of any of these groups; or
   a group of the formula:

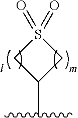

wherein 1 and m are each 0, 1, 2, or 3;
R₆ is absent, hydrogen, or amino; or
   alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, diarylamino_{(C≤12)}, alkyl_{(C≤12)}, cycloalkyl_{(C≤12)}, -alkanediyl_{(C≤12)}-cycloalkyl_{(C≤12)}, -alkanediyl_{(C≤18)}-aralkoxy_{(C≤18)}, heterocycloalkyl_{(C≤12)}, aryl_{(C≤18)}, -arenediyl_{(C≤12)}-alkyl_{(C≤12)}, aralkyl_{(C≤18)}, -arenediyl_{(C≤18)}-heterocycloalkyl_{(C≤12)}, heteroaryl_{(C≤18)}, -heteroarenediyl_{(C≤12)}-alkyl_{(C≤12)}, heteroaralkyl_{(C≤18)}, acyl_{(C≤12)}, alkoxy_{(C≤12)}, or a substituted version of any of these groups; and
X₁ and X₂ are each independently C or N, provided that X₂ is C when R₆ is amino, alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, or diarylamino_{(C≤12)};
   or a pharmaceutically acceptable salt thereof.

In some embodiments, the compound is further defined as:

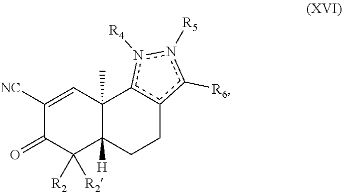

wherein:
R₂ and R₂′ are each independently hydrogen; or
   alkyl_{(C≤12)}, cycloalkyl_{(C≤12)}, alkenyl_{(C≤12)}, alkynyl_{(C≤12)}, aryl_{(C≤18)}, aralkyl_{(C≤18)}, heteroaryl_{(C≤18)}, heteroaralkyl_{(C≤18)}, or a substituted version of any of these groups; or
   R₂ and R₂′ are taken together and are alkanediyl_{(C≤8)}, alkenediyl_{(C≤8)}, or a substituted version of either of these groups;
R₄ and R₅ are each independently absent, hydrogen; or
   alkyl_{(C≤12)}, cycloalkyl_{(C≤12)}, heterocycloalkyl_{(C≤12)}, aryl_{(C≤12)}, aralkyl_{(C≤12)}, heteroaryl_{(C≤12)}, heteroaralkyl_{(C≤12)}, -arenediyl_{(C≤12)}-alkyl_{(C≤12)}, -arenediyl_{(C≤12)}-aryl_{(C≤12)}, -arenediyl_{(C≤12)}-heteroaryl_{(C≤12)}, -arenediyl_{(C≤12)}-heterocycloalkyl_{(C≤12)}, -arenediyl_{(C≤12)}-cycloalkyl_{(C≤12)}, -heteroarenediyl_{(C≤12)}-alkyl_{(C≤12)}, -heteroarenediyl_{(C≤12)}-aryl_{(C≤12)}, -hetero-arenediyl_{(C≤12)}-heteroaryl_{(C≤12)}, -heteroarenediyl_{(C≤12)}-heterocycloalkyl_{(C≤12)}, -heteroarenediyl_{(C≤12)}-cycloalkyl_{(C≤12)}, -heterocyclo-alkanediyl_{(C≤12)}-aryl_{(C≤12)}, -heterocycloalkanediyl_{(C≤12)}-heteroaryl_{(C≤12)}, or a substituted version of any of these groups; or
   a group of the formula:

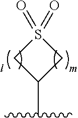

wherein 1 and m are each 0, 1, 2, or 3; and
R₆ is absent, hydrogen; or
   alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, diarylamino_{(C≤12)}, alkyl_{(C≤12)}, cycloalkyl_{(C≤12)}, -alkanediyl_{(C≤12)}-cycloalkyl_{(C≤12)}, -alkanediyl_{(C≤18)}-aralkoxy_{(C≤18)}, heterocycloalkyl_{(C≤12)}, aryl_{(C≤18)}, -arenediyl_{(C≤12)}-alkyl_{(C≤12)}, aralkyl_{(C≤18)}, -arenediyl_{(C≤18)}-heterocycloalkyl_{(C≤12)}, heteroaryl_{(C≤18)}, -heteroarenediyl_{(C≤12)}-alkyl_{(C≤12)}, heteroaralkyl_{(C≤18)}, acyl_{(C≤12)}, alkoxy_{(C≤12)}, or a substituted version of any of these groups;
      or a pharmaceutically acceptable salt thereof.

In some embodiments, the compound is further defined as:

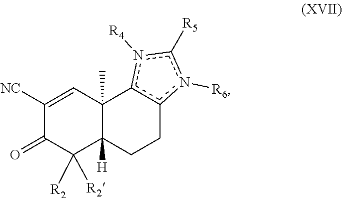

wherein:
R₂ and R₂′ are each independently hydrogen; or
   alkyl_{(C≤12)}, cycloalkyl_{(C≤12)}, alkenyl_{(C≤12)}, alkynyl_{(C≤12)}, aryl_{(C≤18)}, aralkyl_{(C≤18)}, heteroaryl_{(C≤18)}, heteroaralkyl_{(C≤18)}, or a substituted version of any of these groups; or
R₂ and R₂′ are taken together and are alkanediyl_{(C≤8)}, alkenediyl_{(C≤8)}, or a substituted version of either of these groups;
R₄ and R₅ are each independently absent, hydrogen; or
   alkyl_{(C≤12)}, cycloalkyl_{(C≤12)}, heterocycloalkyl_{(C≤12)}, aryl_{(C≤12)}, aralkyl_{(C≤12)}, heteroaryl_{(C≤12)}, heteroaralkyl_{(C≤12)}, -arenediyl_{(C≤12)}-alkyl_{(C≤12)}, -arenediyl_{(C≤12)}-aryl_{(C≤12)}, -arenediyl_{(C≤12)}-heteroaryl_{(C≤12)}, -arenediyl_{(C≤12)}-heterocycloalkyl_{(C≤12)}, -arenediyl_{(C≤12)}-cycloalkyl_{(C≤12)}, -heteroarenediyl_{(C≤12)}-alkyl_{(C≤12)}, -heteroarenediyl_{(C≤12)}-aryl_{(C≤12)}, -hetero-arenediyl_{(C≤12)}-heteroaryl_{(C≤12)}, -heteroarenediyl_{(C≤12)}-heterocycloalkyl_{(C≤12)}, -heteroarenediyl_{(C≤12)}-cycloalkyl_{(C≤12)}, -heterocyclo-alkanediyl_{(C≤12)}-aryl_{(C≤12)}, -heterocycloalkanediyl_{(C≤12)}-heteroaryl_{(C≤12)}, or a substituted version of any of these groups; or
   a group of the formula:

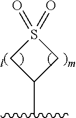

wherein 1 and m are each 0, 1, 2, or 3; and
R₆ is absent, hydrogen; or
   alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, diarylamino_{(C≤12)}, alkyl_{(C≤12)}, cycloalkyl_{(C≤12)}, -alkanediyl_{(C≤12)}-cycloalkyl_{(C≤12)}, -alkanediyl_{(C≤18)}-aralkoxy_{(C≤18)}, heterocycloalkyl_{(C≤12)}, aryl_{(C≤18)}, -arenediyl_{(C≤12)}-alkyl_{(C≤12)}, aralkyl_{(C≤18)}, -arenediyl_{(C≤18)}-heterocycloalkyl_{(C≤12)}, heteroaryl_{(C≤8)}, -heteroarenediyl_{(C≤12)}-alkyl_{(C≤12)}, heteroaralkyl_{(C≤8)}, acyl_{(C≤12)}, alkoxy_{(C≤12)}, or a substituted version of any of these groups;
      or a pharmaceutically acceptable salt thereof.

In some embodiments, the compound is further defined as:

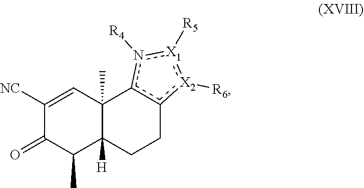

wherein:
R₄ and R₅ are each independently absent, hydrogen; or
   alkyl_{(C≤12)}, cycloalkyl_{(C≤12)}, heterocycloalkyl_{(C≤12)}, aryl_{(C≤12)}, aralkyl_{(C≤12)}, heteroaryl_{(C≤12)}, heteroaralkyl_{(C≤12)}, -arenediyl_{(C≤12)}-alkyl_{(C≤12)}, -arenediyl_{(C≤12)}-aryl_{(C≤12)}, -arenediyl_{(C≤12)}-heteroaryl_{(C≤12)}, -arenediyl_{(C≤12)}-heterocycloalkyl_{(C≤12)}, -arenediyl_{(C≤12)}-cycloalkyl_{(C≤12)}, -heteroarenediyl_{(C≤12)}-alkyl_{(C≤12)}, -heteroarenediyl_{(C≤12)}-aryl_{(C≤12)}, -hetero-arenediyl_{(C≤12)}-heteroaryl_{(C≤12)}, -heteroarenediyl_{(C≤12)}-heterocycloalkyl_{(C≤12)}, -heteroarenediyl_{(C≤12)}-cycloalkyl_{(C≤12)}, -heterocyclo-alkanediyl_{(C≤12)}-aryl_{(C≤12)}, -heterocycloalkanediyl_{(C≤12)}-heteroaryl_{(C≤12)}, or a substituted version of any of these groups; or
   a group of the formula:

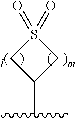

wherein 1 and m are each 0, 1, 2, or 3; and
R₆ is absent, hydrogen; or
   alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, diarylamino_{(C≤12)}, alkyl_{(C≤12)}, cycloalkyl_{(C≤12)}, -alkanediyl_{(C≤12)}-cycloalkyl_{(C≤12)}, -alkanediyl_{(C≤18)}-aralkoxy_{(C≤18)}, heterocycloalkyl_{(C≤12)}, aryl_{(C≤18)}, -arenediyl_{(C≤12)}-alkyl_{(C≤12)}, aralkyl_{(C≤18)}, -arenediyl_{(C≤18)}-heterocycloalkyl_{(C≤12)}, heteroaryl_{(C≤18)}, -heteroarenediyl_{(C≤12)}-alkyl_{(C≤12)}, heteroaralkyl_{(C≤18)}, acyl_{(C≤12)}, alkoxy_{(C≤12)}, or a substituted version of any of these groups; and
X₁ and X₂ are each independently C or N, provided that X₂ is C when R₆ is amino, alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, or diarylamino_{(C≤12)};
   or a pharmaceutically acceptable salt thereof.

In some embodiments, the compound is further defined as:

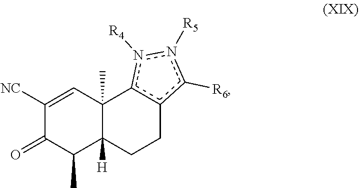

wherein:
R₄ and R₅ are each independently absent, hydrogen; or
   alkyl_{(C≤12)}, cycloalkyl_{(C≤12)}, heterocycloalkyl_{(C≤12)}, aryl_{(C≤12)}, aralkyl_{(C≤12)}, heteroaryl_{(C≤12)}, heteroaralkyl_{(C≤12)}, -arenediyl_{(C≤12)}-alkyl_{(C≤12)}, -arenediyl_{(C≤12)}-aryl_{(C≤12)}, -arenediyl_{(C≤12)}-heteroaryl_{(C≤12)}, -arenediyl_{(C≤12)}-heterocycloalkyl_{(C≤12)}, -arenediyl_{(C≤12)}-cycloalkyl_{(C≤12)}, -heteroarenediyl_{(C≤12)}-alkyl_{(C≤12)}, -heteroarenediyl_{(C≤12)}-aryl_{(C≤12)}, -hetero-arenediyl_{(C≤12)}-heteroaryl_{(C≤12)}, -heteroarenediyl_{(C≤12)}-heterocycloalkyl_{(C≤12)}, -heteroarenediyl_{(C≤12)}-cycloalkyl_{(C≤12)}, -heterocyclo-alkanediyl_{(C≤12)}-aryl_{(C≤12)}, -heterocycloalkanediyl_{(C≤12)}-heteroaryl_{(C≤12)}, or a substituted version of any of these groups; or
   a group of the formula:

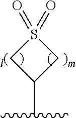

wherein 1 and m are each 0, 1, 2, or 3; and
R₆ is absent, hydrogen; or
   alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, diarylamino_{(C≤12)}, alkyl_{(C≤12)}, cycloalkyl_{(C≤12)}, -alkanediyl_{(C≤12)}-cycloalkyl_{(C≤12)}, -alkanediyl_{(C≤18)}-aralkoxy_{(C≤18)}, heterocycloalkyl_{(C≤12)}, aryl_{(C≤18)}, -arenediyl_{(C≤12)}-alkyl_{(C≤12)}, aralkyl_{(C≤18)}, -arenediyl_{(C≤18)}-heterocycloalkyl_{(C≤12)}, heteroaryl_{(C≤18)}, -heteroarenediyl_{(C≤12)}-alkyl_{(C≤12)}, heteroaralkyl_{(C≤18)}, acyl_{(C≤12)}, alkoxy_{(C≤12)}, or a substituted version of any of these groups;
      or a pharmaceutically acceptable salt thereof.

In some embodiments, the compound is further defined as:

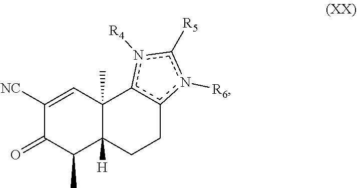

wherein:
R₄ and R₅ are each independently absent, hydrogen; or
   alkyl_{(C≤12)}, cycloalkyl_{(C≤12)}, heterocycloalkyl_{(C≤12)}, aryl_{(C≤12)}, aralkyl_{(C≤12)}, heteroaryl_{(C≤12)}, heteroaralkyl_{(C≤12)}, -arenediyl_{(C≤12)}-alkyl_{(C≤12)}, -arenediyl_{(C≤12)}-aryl_{(C≤12)}, -arenediyl_{(C≤12)}-heteroaryl_{(C≤12)}, -arenediyl_{(C≤12)}-heterocycloalkyl_{(C≤12)}, -arenediyl_{(C≤12)}-cycloalkyl_{(C≤12)}, -heteroarenediyl_{(C≤12)}-alkyl_{(C≤12)}, -heteroarenediyl_{(C≤12)}-aryl_{(C≤12)}, -hetero-arenediyl_{(C≤12)}-heteroaryl_{(C≤12)}, -heteroarenediyl_{(C≤12)}-heterocycloalkyl_{(C≤12)}, -heteroarenediyl_{(C≤12)}-cycloalkyl_{(C≤12)}, -heterocyclo-alkanediyl_{(C≤12)}-aryl_{(C≤12)}, -heterocycloalkanediyl_{(C≤12)}-heteroaryl_{(C≤12)}, or a substituted version of any of these groups; or
   a group of the formula:

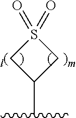

wherein 1 and m are each 0, 1, 2, or 3; and
R₆ is absent, hydrogen; or
   alkylamino_{(C≤12)}, dialkylamino_{(C≤12)}, cycloalkylamino_{(C≤12)}, dicycloalkylamino_{(C≤12)}, alkyl(cycloalkyl)amino_{(C≤12)}, arylamino_{(C≤12)}, diarylamino_{(C≤12)}, alkyl_{(C≤12)}, cycloalkyl_{(C≤12)}, -alkanediyl_{(C≤12)}-cycloalkyl_{(C≤12)}, -alkanediyl_{(C≤18)}-aralkoxy_{(C≤18)}, heterocycloalkyl_{(C≤12)}, aryl_{(C≤18)}, -arenediyl_{(C≤12)}-alkyl_{(C≤12)}, aralkyl_{(C≤18)}, -arenediyl_{(C≤8)}-heterocycloalkyl_{(C≤12)}, heteroaryl_{(C≤8)}, -heteroarenediyl_{(C≤12)}-alkyl_{(C≤12)}, heteroaralkyl_{(C≤18)}, acyl_{(C≤12)}, alkoxy_{(C≤12)}, or a substituted version of any of these groups;
      or a pharmaceutically acceptable salt thereof.

In some embodiments, X₁ and X₂ are both N. In other embodiments, X₁ and X₂ are not both N. In some embodiments, either X₁ or X₂ is N. In some embodiments, X₁ is N. In some embodiments, X₂ is N. In some embodiments, R₃ is alkyl_{(C≤12)} or substituted alkyl_{(C≤12)}. In further embodiments, R₃ is alkyl_{(C≤12)}, such as methyl. In some embodiments, R₁ is cyano. In other embodiments, R₁ is —C(O)Rₐ. In some embodiments, Rₐ is alkoxy_{(C≤6)}, such as methoxy. In other embodiments, Rₐ is amino. In some embodiments, R₂′ is hydrogen. In some embodiments, R₂ is hydrogen. In some embodiments, R₂′ and R₂ are both hydrogen. In other embodiments, R₂ is alkyl_{(C≤12)} or substituted alkyl_{(C≤12)}. In further embodiments, R₂ is alkyl_{(C≤12)}, such as methyl.

In some embodiments, R₄ is absent. In other embodiments, R₄ is hydrogen. In still other embodiments, R₄ is alkyl_{(C≤12)} or substituted alkyl_{(C≤12)}. In further embodiments, R₄ is substituted alkyl_{(C≤12)}, such as R₄ is 2,2,2-trifluoroethyl. In still other embodiments, R₄ is cycloalkyl_{(C≤12)} or substituted cycloalkyl_{(C≤12)}. In further embodiments, R₄ is cycloalkyl_{(C≤12)}, such as cyclohexyl. In yet other embodiments, R₄ is heterocycloalkyl_{(C≤12)} or substituted heterocycloalkyl_{(C≤12)}. In further embodiments, R₄ is heterocycloalkyl_{(C≤12)}, such as tetrahydro-2H-pyran-4-yl or 1,1-dioxidotetrahydrothiophen-3-yl. In other embodiments, R₄ is:

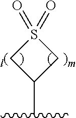

wherein:
l and m are each 0, 1, 2, or 3.
   In some embodiments, 1 is 1 or 2. In some embodiments, m is 1 or 2. In still other embodiments, R₄ is aryl_{(C≤18)}, heteroaryl_{(C≤18)}, or a substituted version of either of these groups. In further embodiments, R₄ is aryl_{(C≤18)} or substituted aryl_{(C≤18)}. In still further embodiments, R₄ is aryl_{(C≤18)}, such as phenyl, o-tolyl, p-tolyl, [1,1′-biphenyl]-4-yl, 4-isopropylphenyl, naphthalen-1-yl, 4′-methyl-[1,1′-biphenyl]-4-yl, or 2′-methyl-[1,1′-biphenyl]-4-yl. In yet other embodiments, R₄ is substituted aryl_{(C≤18)}, such as 4-(trifluoromethyl)phenyl, 4-cyanophenyl, 2-fluorophenyl, 4-fluorophenyl, 4-chlorophenyl, 3,4-dichlorophenyl, 4-methoxyphenyl, 4-(trifluoromethoxy)phenyl, 4-carboxyphenyl, 4′-methoxy-[1,1′-biphenyl]-4-yl, 4′-(dimethylamino)-[1,1′-biphenyl]-4-yl, 2′-fluoro-[1,1′-biphenyl]-4-yl, 3′-fluoro-[1,1′-biphenyl]-4-yl, 2′-(hydroxymethyl)-[1,1′-biphenyl]-4-yl, 3′-(hydroxymethyl)-[1,1′-biphenyl]-4-yl, 4′-(hydroxymethyl)-[1,1′-biphenyl]-4-yl, or 5-(3-(hydroxymethyl)phenyl. In other embodiments, R₄ is aralkyl_{(C≤18)} or substituted aralkyl_{(C≤8)}. In further embodiments, R₄ is aralkyl_{(C≤18)}, such as benzyl.
   In still other embodiments, R₄ is -arenediyl_{(C≤12)}-heterocycloalkyl_{(C≤12)} or substituted -arenediyl_{(C≤12)}-heterocycloalkyl_{(C≤12)}. In further embodiments, R₄ is -arenediyl_{(C≤12)}-heterocycloalkyl_{(C≤12)}, such as 4-morpholinophenyl. In other embodiments, R₄ is heteroaryl_{(C≤18)} or substituted heteroaryl_{(C≤18)}. In further embodiments, R₄ is heteroaryl_{(C≤18)}, such as pyridin-4-yl, quinolin-4-yl, 5-methylpyrindin-2-yl, 6-methylpyrindin-3-yl, (pyridin-3-yl)phenyl, (pyridin-4-yl)phenyl, 4-(3,5-dimethylisoxazol-4-yl)phenyl, 4-(pyrimidin-4-yl)phenyl, 4-(pyrimidin-5-yl)phenyl, 4-(pyridin-3-yl)phenyl, 4-(pyridin-4-yl)phenyl, 5-phenylpyridin-2-yl, [3,3′-bipyridin]-6-yl, 5-cyclopropylpyridin-2-yl, 6-phenylpyridin-3-yl, 4-(6-methylpyridazin-4-yl)phenyl, 5-methyl-1,2,4-oxadiazol-3-yl, 3-methyl-1,2,4-oxadiazol-5-yl, 4-methyl-5-phenyl-4H-1,2,4-triazol-3-yl, 1-phenylpiperidin-4-yl, 4-phenyloxazol-2-yl, 4-(6-methylpyridazin-4-yl)phenyl, 4-(5-methyl-1,2,4-oxadiazol-3-yl)phenyl, 4-(3-methyl-1,2,4-oxadiazol-5-yl)phenyl, 4-(1,2,4-oxadiazol-3-yl)phenyl, 4-(pyridazin-3-yl)phenyl, 4-(5-methylpyridazin-3-yl)phenyl, 1-methyl-1H-benzo[d]imidazol-2-yl, or benzo[d]thiazol-2-yl. In still other embodiments, R₄ is substituted heteroaryl_{(C≤18)}, such as 2-fluoro-4-(pyridin-3-yl)phenyl, 5-(trifluoromethyl)pyridin-2-yl, 5-(3-fluorophenyl)pyridin-2-yl, 5-(4-fluorophenyl)pyridin-2-yl, 4-(2-(hydroxymethyl)pyridine-4-yl)phenyl, 4-(2-(fluoromethyl)pyridine-4-yl)phenyl, 5-(trifluoromethyl)benzo[d]oxazol-2-yl, 6-chlorobenzo[d]thiazol-2-yl, or 4-(5-fluoropyridin-3-yl)phenyl.

In some embodiments, R₅ is absent. In other embodiments, R₅ is hydrogen. In still other embodiments, R₅ is alkyl_{(C≤12)} or substituted alkyl_{(C≤12)}. In further embodiments, R₅ is alkyl_{(C≤12)}, such as methyl. In yet other embodiments, R₅ is cycloalkyl_{(C≤12)} or substituted cycloalkyl_{(C≤12)}. In further embodiments, R₅ is cycloalkyl_{(C≤12)}, such as cyclohexyl. In other embodiments, R₅ is heterocycloalkyl_{(C≤12)} or substituted heterocycloalkyl_{(C≤12)}. In further embodiments, R₅ is heterocycloalkyl_{(C≤12)}, such as tetrahydro-2H-pyran-4-yl. In still other embodiments, R₅ is aryl_{(C≤18)} or substituted aryl_{(C≤18)}. In further embodiments, R₅ is aryl_{(C≤18)}, such as phenyl, o-tolyl, p-tolyl, [1,1′-biphenyl]-4-yl, 4-isopropylphenyl, naphthalen-1-yl, [1,1′-biphenyl]-3-yl, or 3-isopropylphenyl.

In yet other embodiments, R₅ is substituted aryl_{(C≤18)}, such as 4-chlorophenyl, 3,4-dichlorophenyl, 4-methoxyphenyl, 4-(trifluoromethoxy)phenyl, 3-bromophenyl, 3-chlorophenyl, 4-(dimethylamino)phenyl. In other embodiments, R₅ is aralkyl_{(C≤18)} or substituted aralkyl_{(C≤8)}. In further embodiments, R₅ is aralkyl_{(C≤18)}, such as R₅ is benzyl. In still other embodiments, R₅ is -arenediyl_{(C≤12)}-heterocycloalkyl_{(C≤12)} or substituted -arenediyl_{(C≤12)}-heterocycloalkyl_{(C≤12)}. In further embodiments, R₅ is -arenediyl_{(C≤12)}-heterocycloalkyl_{(C≤12)}, such as 3-morpholinophenyl. In yet other embodiments, R₅ is heteroaryl_{(C≤18)} or substituted heteroaryl_{(C≤8)}. In further embodiments, 10 heteroaryl_{(C≤18)}, such as pyridin-4-yl, quinolin-4-yl, quinolin-3-yl, quinolin-5-yl, 3-(pyridin-4-yl)phenyl, 3-(pyrimidin-5-yl)phenyl, 2-isopropylpyrimidin-5-yl, 6-cyclopropylpyridin-3-yl, 3-(1-methyl-1H-pyrazol-4-yl)phenyl, 1-methyl-1H-pyrazol-4-yl, 3-(3,5-dimethylisoxazol-4-yl)phenyl, 3-(1-methyl-1H-pyrazol-5-yl)phenyl, or 2-cyclopropylpyridin-4-yl. In other embodiments, R₅ is substituted heteroaryl_{(C≤18)}, such as 2-(trifluoromethyl)pyridin-4-yl or 3-(5-fluoropyridin-3-yl)phenyl. In still other embodiments, R₅ is -heteroarenediyl_{(C≤12)}-heterocycloalkyl_{(C≤12)} or substituted -heteroarenediyl_{(C≤12)}-heterocycloalkyl_{(C≤12)}. In further embodiments, R₅ is -heteroarenediyl_{(C≤12)}-heterocycloalkyl_{(C≤12)}, such as 2-morpholinopyridin-4-yl.

In some embodiments, R₆ is hydrogen. In other embodiments, R₆ is aryl_{(C≤18)}, heteroaryl_{(C≤18)}, or a substituted version of either of these groups. In still other embodiments, R₆ is amino. In yet other embodiments, R₆ is alkylamino_{(C≤12)} or substituted alkylamino_{(C≤12)}. In further embodiments, R₆ is alkylamino_{(C≤12)}, such as methylamino. In other embodiments, R₆ is cycloalkylamino_{(C≤12)} or substituted cycloalkylamino_{(C≤12)}. In further embodiments, R₆ is cycloalkylamino_{(C≤12)}, such as cyclobutylamino. In still other embodiments, R₆ is alkyl(cycloalkyl)amino_{(C≤12)} or substituted alkyl(cycloalkyl)amino_{(C≤12)}. In further embodiments, R₆ is alkyl(cycloalkyl)amino_{(C≤12)}, such as methyl(cyclobutyl)amino. In yet other embodiments, R₆ is arylamino_{(C≤12)} or substituted arylamino_{(C≤12)}. In further embodiments, R₆ is arylamino_{(C≤12)}, such as phenylamino. In other embodiments, R₆ is alkyl_{(C≤12)} or substituted alkyl_{(C≤12)}. In further embodiments, R₆ is alkyl_{(C≤12)}, such as methyl. In still other embodiments, R₆ is substituted alkyl_{(C≤12)}, such as 2-hydroxyethyl or 2-methoxyethyl. In yet other embodiments, R₆ is acyl_{(C≤6)} or substituted acyl_{(C≤6)}. In further embodiments, R₆ is acyl_{(C≤6)}, such as —C(O)CH₃. In other embodiments, R₆ is cycloalkyl_{(C≤12)} or substituted cycloalkyl_{(C≤12)}. In further embodiments, R₆ is cycloalkyl_{(C≤12)}, such as cyclopropyl or cyclohexyl. In still other embodiments, R₆ is -alkanediyl_{(C≤18)}-cycloalkyl_{(C≤18)} or substituted -alkanediyl_{(C≤18)}-cycloalkyl_{(C≤18)}. In further embodiments, R₆ is -alkanediyl_{(C≤8)}-cycloalkyl_{(C≤8)}, such as cyclobutylmethyl. In yet other embodiments, R₆ is aryl_{(C≤18)} or substituted aryl_{(C≤18)}. In further embodiments, R₆ is aryl_{(C≤18)}, such as phenyl, o-tolyl, p-tolyl, or 3-isopropylphenyl. In other embodiments, R₆ is substituted aryl_{(C≤18)}, such as 2-fluorophenyl, 4-fluorophenyl, 4-(hydroxymethyl)phenyl, 3-fluorophenyl, or 4-(fluoromethyl)phenyl. In still other embodiments, R₆ is aralkyl_{(C≤18)} or substituted aralkyl_{(C≤18)}. In further embodiments, R₆ is aralkyl_{(C≤18)}, such as benzyl. In yet other embodiments, R₆ is substituted aralkyl_{(C≤18)}, such as 2-fluorobenzyl, 4-fluorobenzyl, or 4-chlorobenzyl. In other embodiments, R₆ is -arenediyl_{(C≤18)}-heterocycloalkyl_{(C≤12)} or substituted -arenediyl_{(C≤18)}-heterocycloalkyl_{(C≤12)}. In further embodiments, R₆ is -arenediyl_{(C≤18)}-heterocycloalkyl_{(C≤12)}, such as 4-morpholinophenyl. In still other embodiments, R₆ is heteroaryl_{(C≤18)} or substituted heteroaryl_{(C≤18)}. In further embodiments, R₆ is heteroaryl_{(C≤18)}, such as pyridin-2-yl, pyridin-3-yl, pyridin-4-yl, 2-methyl-2H-tetrazol-5-yl, 1-methyl-1H-pyrazol-4-yl, pyrimidin-4-yl, pyrimidin-5-yl, 3-methyl-1,2,4-oxadiazol-5-yl)phenyl, pyridin-2-ylmethyl, 3-methyl-1,2,4-oxadiazol-5-yl, or 5-methyl-1,2,4-oxadiazol-3-yl. In still other embodiments, R₆ is heteroaralkyl_{(C≤18)} or heteroaralkyl_{(C≤8)}. In further embodiments, R₆ is heteroaralkyl_{(C≤18)}, such as 2-pyridinylmethyl or 4-pyridinylmethyl. In yet other embodiments, R₆ is -alkanediyl_{(C≤8)}-aralkoxy_{(C≤8)} or substituted -alkanediyl_{(C≤8)}-aralkoxy_{(C≤8)}. In further embodiments, R₆ is -alkanediyl_{(C≤18)}-aralkoxy_{(C≤18)}, such as 2-(benzyloxy)ethyl.

In some embodiments, the compound is further defined as:

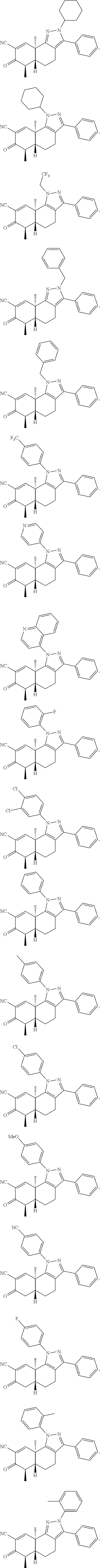

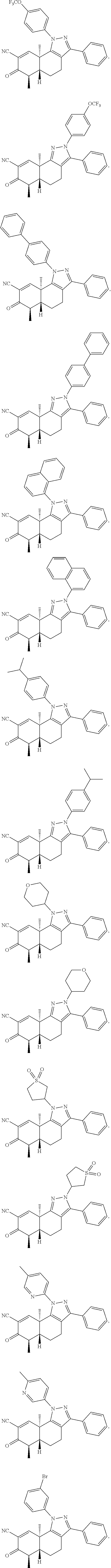

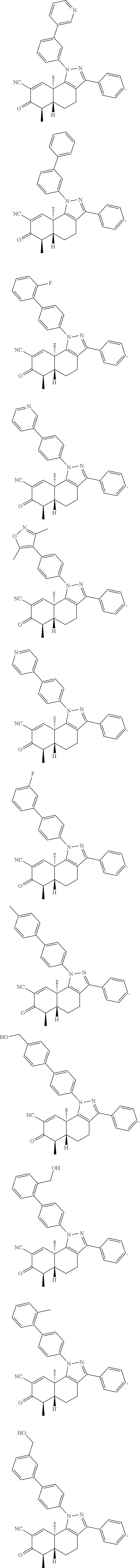

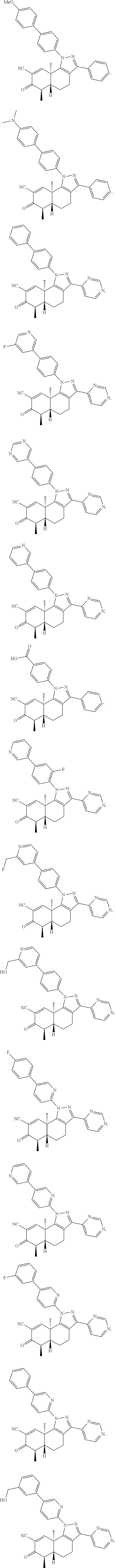

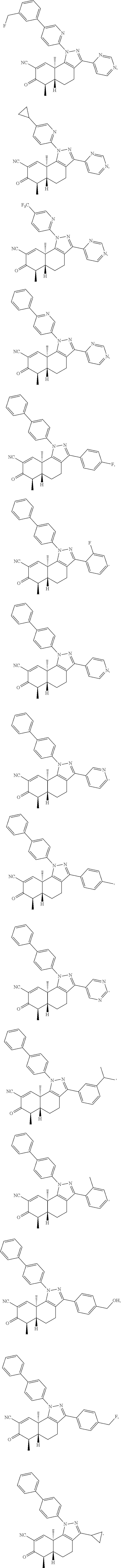

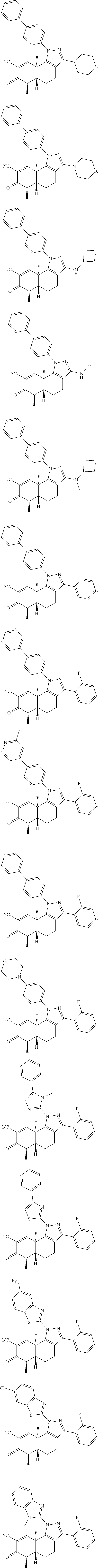

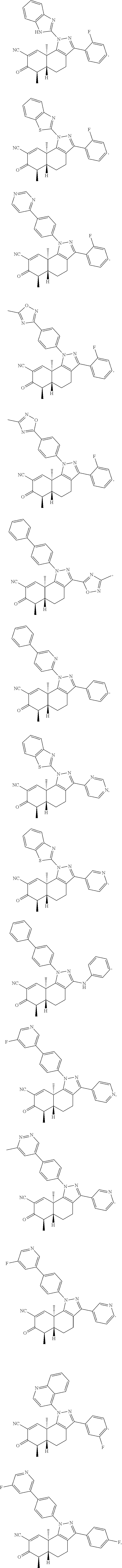

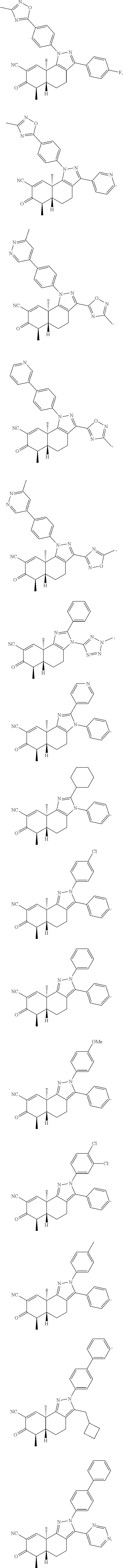

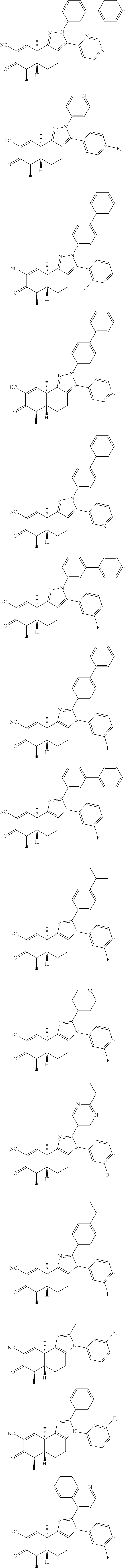

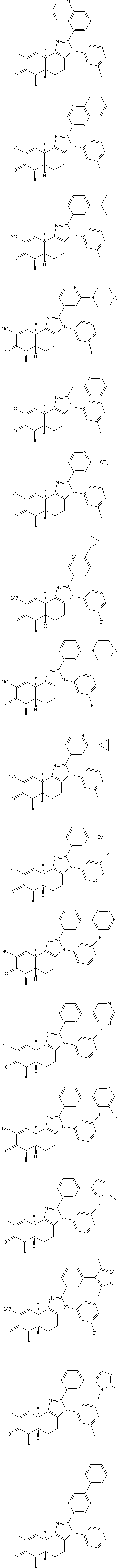

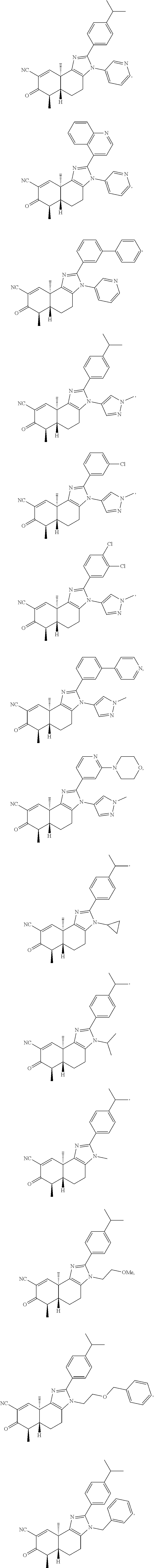

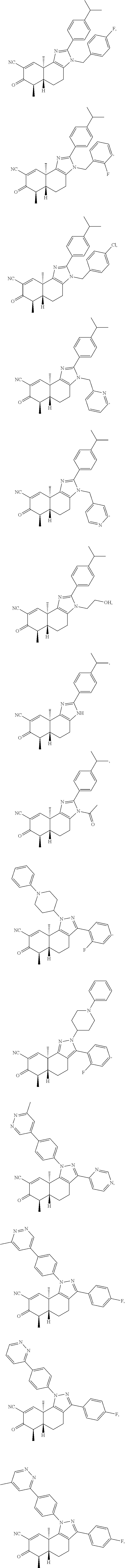

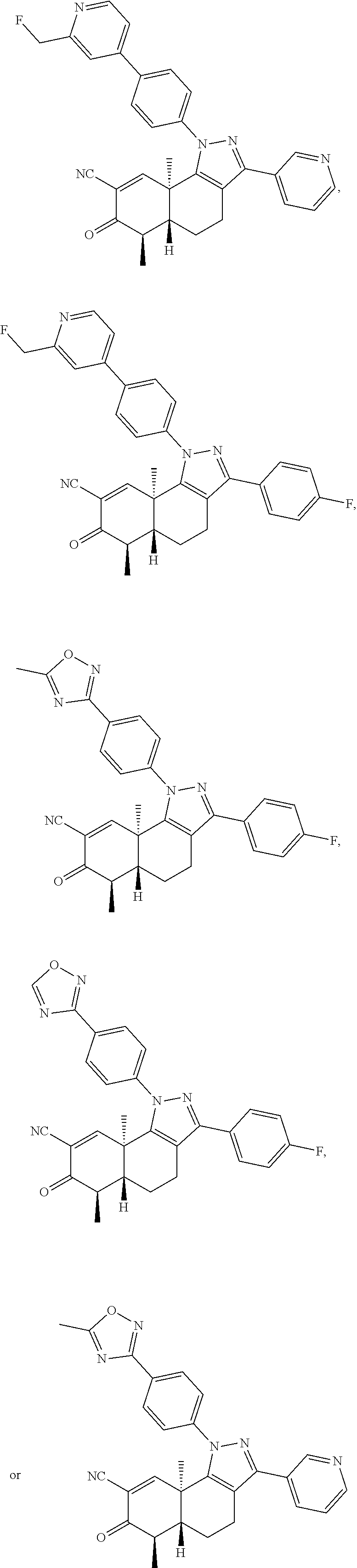 [SMILES: C[C@H]1C(=O)C(C#N)=C[C@@]2(C)c3c(c(-c4cccnc4)nn3-c3ccc(-c4ccnc(CF)c4)cc3)CC[C@H]12 ; C[C@H]1C(=O)C(C#N)=C[C@@]2(C)c3c(c(-c4ccc(F)cc4)nn3-c3ccc(-c4ccnc(CF)c4)cc3)CC[C@H]12 ; Cc1nc(-c2ccc(-n3nc(-c4ccc(F)cc4)c4c3[C@]3(C)C=C(C#N)C(=O)[C@H](C)[C@H]3CC4)cc2)no1 ; C[C@H]1C(=O)C(C#N)=C[C@@]2(C)c3c(c(-c4ccc(F)cc4)nn3-c3ccc(-c4ncon4)cc3)CC[C@H]12 ; Cc1nc(-c2ccc(-n3nc(-c4cccnc4)c4c3[C@]3(C)C=C(C#N)C(=O)[C@H](C)[C@H]3CC4)cc2)no1]

or a pharmaceutically acceptable salt of any of the above formulas.

In some embodiments, the compound is further defined as:

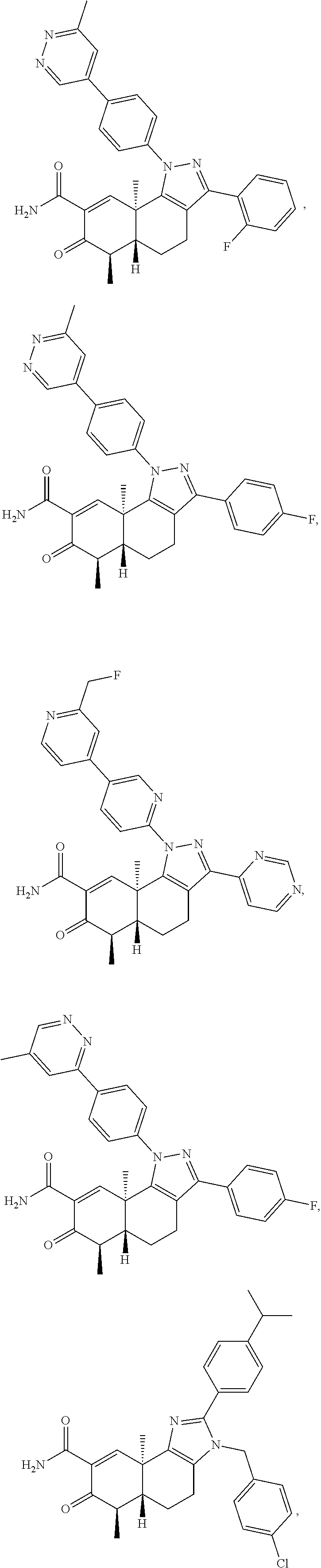

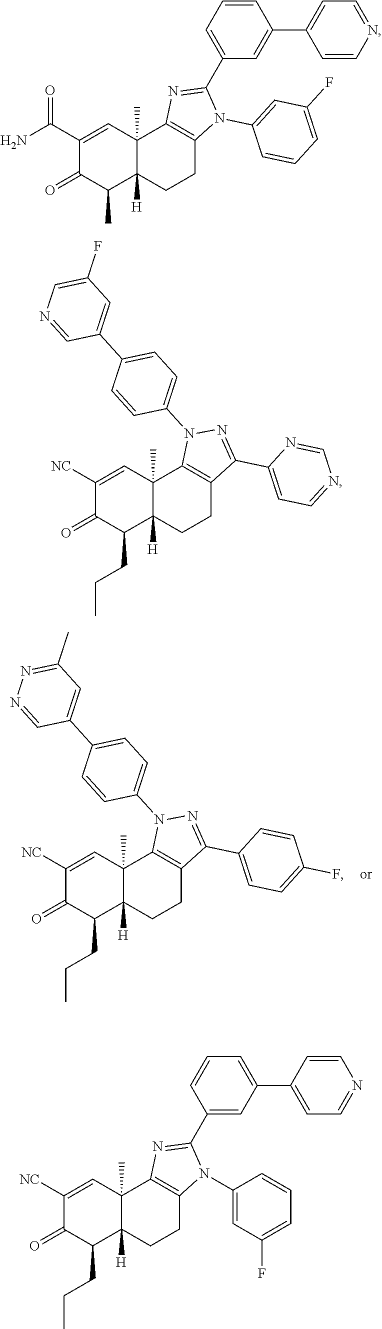

or a pharmaceutically acceptable salt of any of the above formulas.

In some embodiments, the therapeutically effective amount is a daily dose is 0.01-100 mg of inverse agonist per kg of body weight. In some embodiments, the daily dose is 0.05-30 mg of inverse agonist per kg of body weight. In some embodiments, the daily dose is 0.1-10 mg of inverse agonist per kg of body weight. In some embodiments, the daily dose is 0.1-5 mg of inverse agonist per kg of body weight. In some embodiments, the daily dose is 0.1-2.5 mg of inverse agonist per kg of body weight.

In some embodiments, the inverse agonist is administered as a single dose to the patient per day. In other embodiments, the inverse agonist is administered as two or more doses to the patient per day. In some embodiments, the inverse agonist is administered orally, intraarterially or intravenously. In some embodiments, the inverse agonist is formulated as a hard or soft capsule or a tablet.

In still another aspect, the present disclosure provides compounds of the formula:

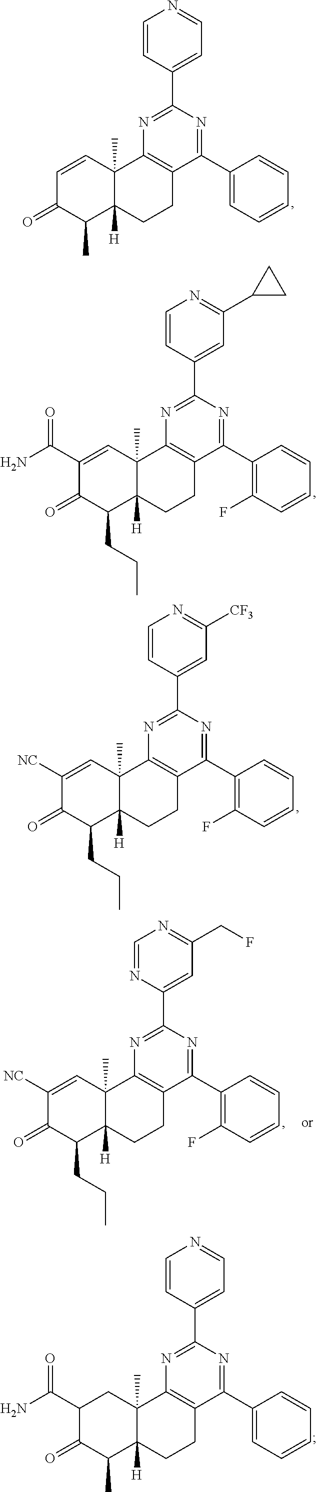

or a pharmaceutically acceptable salt thereof.

In yet another aspect, the present disclosure provides pharmaceutical compositions comprising a compound of the present disclosure and an excipient.

In some embodiments, the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion. In some embodiments, the pharmaceutical composition is formulated for oral administration. In other embodiments, the pharmaceutical composition is formulated for administration via injection such as for intraarterial administration, intramuscular administration, intraperitoneal administration, or intravenous administration. In other embodiments, the pharmaceutical composition is formulated for administration topically such as for topical administration to the skin or to the eye. In some embodiments, the pharmaceutical composition is formulated as a unit dose.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B show the effect of compound 14 on RORα, RORβ, and RORγt activity.

FIGS. 2A-2D show the effect of compound 14 on Th17 cell differentiation (A) and suppression of TH17 signature genes (B-D).

FIGS. 3A-3B show activity of compound 14 in the mCIA model dosed orally at 3 mg/kg.

### DESCRIPTION OF ILLUSTRATIVE EMBODIMENTS

Disclosed herein are methods, compounds, and compositions that may be used to inhibit or modulate the activity of the RORγ nuclear receptor and/or IL-17 by covalent modification of the allosteric binding site. These methods are thus useful in the treatment of a wide variety of different indications such as autoimmune disease, metabolic diseases, cancer, and infections. These methods may be used to modulate the expression of one or more downstream compound such as interleukin-17 (IL-17) through covalent modification of the RORγ nuclear receptor.

### I. Compounds and Synthetic Methods

The compounds of the present invention and the compounds for use with the present invention (also referred to as “compounds of the present disclosure”) are shown, for example, above, in the summary of the invention section, and in the claims below. They may be made using the synthetic methods described in PCT/US2017/000094 (WO 2018/111315) and PCT/US2019/037543, the entire contents of which are hereby incorporated by reference. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All the compounds of the present invention may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present invention have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present invention may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration. In some embodiments, the present compounds may contain two or more atoms which have a defined stereochemical orientation.

Chemical formulas used to represent compounds of the present invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present invention function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present invention exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

### II. RORγ and Cytokine IL-17

Without being bound by theory, the presence of a Michael acceptor (MA) in the A ring is important in methods of modulating the activity of RORγ/RORγt described herein. In particular, the presence of an electron-withdrawing group at C2 shows strong activity, with a CN group at C2 showing stronger activity. This relationship suggests that a cysteine dependent covalent drug:protein interaction is involved in the activity of these inhibitors and of related compounds. This mechanism of action (MOA) has been explored successfully with other protein classes, most notably with kinases, nonetheless it is rare in the nuclear hormone receptor field, and unreported in the ROR family. The chemical biology literature of NHR's provides few insights on how to triage the eight cysteines on ROR7 for their proclivity towards covalent drug:protein interaction. PPARγ is the lone exception, where it has been noted that several endogenous fatty acid metabolite ligands, e.g. PGJ₂, bind in the orthosteric site through covalent interaction with cysteine 285 on helix H3 and thusly regulate the transcriptional activity. Interestingly, cysteine 320 on alpha helix H3 of RORγt is in the same location as cysteine 285 on PPARγ. Based on this evidence suggesting that cysteine 320 of ROR7 (see SEQ ID NO: 1; and which is equivalent to cysteine 299 in RORγt (see SEQ ID NO: 2)) could be the companion residue of interest, site directed mutation of cysteine 320 to alanine and serine was used to obtain functional proteins that could be used to test this hypothesis. The activities of compounds 14 and 21 were found to be unaffected by both mutations in the full-length RORγt RORE-luciferase reporter assay in Jurkat cells, i.e., their activity was maintained in the presence of these mutations. These observations were supported by competitive binding studies monitored by mass spectrometry that demonstrated that 14 and 21 did not compete with cholesterol sulfate, a known orthosteric ligand for RORγ/RORγt. Reporter gene constructs wherein each of the remaining seven cysteine residues in the LBD were selectively mutated to a different amino acid residue were prepared. When compounds 14 and 21 were assayed against these constructs only the cysteine 476 in ROR7 (cysteine 455 in RORγt) mutations led to a reduction in RORγt RORE-luciferase reporter expression in Jurkat cells versus the parent construct, demonstrating that this cysteine was the singular residue involved in covalent interactions with the Michael acceptor moiety.

-RORγ Protein Sequence:
SEQ ID NO: 1
MDRAPQRQHR ASRELLAAKK THTSQIEVIP CKICGDKSSG
IHYGVITCEG CKGFFRRSQR CNAAYSCTRQ QNCPIDRTSR
NRCQHCRLQK CLALGMSRDA VKFGRMSKKQ RDSLHAEVQK
QLQQRQQQQQ EPVVKTPPAG AQGADTLTYT LGLPDGQLPL
GSSPDLPEAS ACPPGLLKAS GSGPSYSNNL AKAGLNGASC
HLEYSPERGK AEGRESFYST GSQLTPDRCG LRFEEHRHPG
LGELGQGPDS YGSPSFRSTP EAPYASLTEI EHLVQSVCKS
YRETCQLRLE DLLRQRSNIF SREEVTGYQR KSMWEMWERC
AHHLTEAIQY VVEFAKRLSG FMELCQNDQI VLLKAGAMEV
VLVRMCRAYN ADNRTVFFEG KYGGMELFRA LGCSELISSI
FDFSHSLSAL HFSEDEIALY TALVLINAHR PGLQEKRKVE
QLQYNLELAF HHHLCKTHRQ SILAKLPPKG KLRSLCSQHV
ERLQIFQHLH PIVVQAAFPP LYKELFSTET ESPVGLSK
-RORγt Protein Sequence:
SEQ ID NO: 2
MRTQIEVIPC KICGDKSSGI HYGVITCEGC KGFFRRSQRC
NAAYSCTRQQ NCPIDRTSRN RCQHCRLQKC LALGMSRDAV
KFGRMSKKQR DSLHAEVQKQ LQQRQQQQQE PVVKTPPAGA
QGADTLTYTL GLPDGQLPLG SSPDLPEASA CPPGLLKASG
SGPSYSNNLA KAGLNGASCH LEYSPERGKA EGRESFYSTG
SQLTPDRCGL RFEEHRHPGL GELGQGPDSY GSPSFRSTPE
APYASLTEIE HLVQSVCKSY RETCQLRLED LLRQRSNIFS
REEVTGYQRK SMWEMWERCA HHLTEAIQYV VEFAKRLSGF
MELCQNDQIV LLKAGAMEVV LVRMCRAYNA DNRTVFFEGK
YGGMELFRAL GCSELISSIF DFSHSLSALH FSEDEIALYT
ALVLINAHRP GLQEKRKVEQ LQYNLELAFH HHLCKTHRQS
ILAKLPPKGK LRSLCSQHVE RLQIFQHLHP IVVQAAFPPL
YKELFSTETE SPVGLSK

### B. Cytokine IL-17

Various reports have implicated the inflammatory cytokine IL-17 in the pathogenesis of many autoimmune diseases, including rheumatoid arthritis, psoriasis and psoriatic arthritis, inflammatory bowel diseases (including but not limited to Crohn's disease), multiple sclerosis, autoimmune nephritis, autoimmune uveitis, Type 1 diabetes, and ankylosing spondylitis. In some embodiments, the compounds provided herein may be administered to a patient in order to treat or prevent one or more of these diseases or disorders. A type of T lymphocyte known as a Th17 cell is a primary source of IL-17. There are multiple members of the IL-17 family. The first identified member, IL-17A, is commonly referred to as IL-17. IL-17 is composed of two monomers linked by disulfide bonds to form a homodimer (Miossec and Kolls, 2012). Aside from IL-17A, the other principal family member is IL-17F. Some evidence suggests that IL-17F and IL-17A, though they have many effects in common, may have different effects in certain settings such as lung inflammation. The IL-17 cytokines bind to IL-17 receptors (IL-17R) located in the membrane of select cell types. Although there are multiple subtypes of the IL-17 receptor, the IL-17RA/IL-17RC complex is required for the activity of IL-17A and IL-17F. IL-17RA has the unusual property of signaling through a pathway that involves an adaptor protein (ACT1) rather than the Janus kinase/signal transducer and activator of transcription (JAK/STAT) pathway employed by most interleukin receptors. Binding of IL-17A to IL-17RA activates the pro-inflammatory nuclear factor-kappa B (NF-κB) pathway and pro-inflammatory elements of the mitogen-activated protein kinase (MAPK) pathway such as JUN N-terminal kinase (JNK), p38 and extracellular signal-related kinase (ERK). IL-17 activity stimulates secretion of IL-6 and IL-8 from mesenchymal cells and leads to fever along with the accumulation of neutrophils in blood and tissue. In some embodiments, the compounds provided herein may be used to inhibit the secretion of IL-6 and IL-8 from mesenchymal cells. In some embodiments, the compounds provided herein may be administered to a patient in order to prevent or inhibit fever in a patient. In some embodiments, the compounds provided herein may be administered to a patient in order to prevent the accumulation of neutrophils in the blood or tissue of the patient.

Aside from its contribution to acute inflammation, IL-17 also contributes to chronic inflammation (Miossec and Kolls, 2012). In some embodiments, the compounds provided herein may be administered to a patient in order to prevent or treat chronic inflammation. IL-17 stimulates the production of matrix metalloproteinases (MMPs), which among other effects can degrade cartilage in joints. In some embodiments, the compounds provided herein may be administered to a patient in order to prevent or treat degradation of the patient's cartilage. IL-17 also increases the expression of receptor activator of NF-κB ligand (RANKL) in osteoblasts, leading to differentiation and activation of osteoclasts and bone degradation. In some embodiments, the compounds provided herein may be administered to a patient in order to prevent or treat degradation of the patient's bone. Depending on the target cell that is exposed to it, IL-17 may stimulate the production of IL-6, IL-8, IL-1, tumor necrosis factor (TNF), MMPs, nitric oxide, or several other proteins that are implicated in inflammatory conditions (e.g., tissue factor, CCL20, G-CSF and GM-CSF). In some embodiments, the compounds provided herein may be administered to a patient in order to inhibit the production of IL-6, IL-8, IL-1, tumor necrosis factor (TNF), MMPs, nitric oxide, or several other proteins that are implicated in inflammatory conditions (e.g., tissue factor, CCL20, G-CSF and GM-CSF).

Although IL-17 plays a role in the immune response to invading pathogens, excessive IL-17 activity has been implicated in pathologies associated with an excessive immune response to an infection. In some embodiments, the compounds provided herein may be administered to a patient in order to prevent or treat excessive immune response to an infection. For example, IL-17 has been implicated in the severe neuroinflammation associated with Toxoplasma gondii infection and increased severity of lesions associated with Leishmania infection. In some embodiments, the compounds provided herein may be administered to a patient in order to treat or prevent neuroinflammation, for example, neuroinflammation associated with Toxoplasma gondii infection. In some embodiments, the compounds provided herein may be administered to a patient in order to treat or prevent lesions associated with Leishmania infection. In these and other cases, IL-17 appears to play a role in perpetuating the infection, promoting an excessive inflammatory response, and inhibiting clearance of the infectious agent (Waite and Skokos, 2012). In some embodiments, the compounds provided herein may be administered to a patient in order to prevent an excessive inflammatory response and/or promote the clearance of an infectious agent.

Drugs targeting IL-17 have entered clinical trials for a wide variety of inflammatory conditions, including psoriasis, rheumatoid arthritis, ankylosing spondylitis, uveitis, Behcet's disease, psoriatic arthritis, Crohn's disease, polymyalgia rheumatica, dry eye syndrome, multiple sclerosis, graft-versus-host disease, and asthma. In some embodiments, the compounds provided herein may be administered to a patient in order to treat or prevent one or more of these diseases or disorders. Preclinical evidence also implicates IL-17 in the pathology of type 1 diabetes, and Th17 cells are elevated in patients with adult onset Still's disorder, another autoimmune disease. In some embodiments, the compounds provided herein may be administered to a patient in order to treat type 1 diabetes. In some embodiments, the compounds provided herein may be administered to a patient in order to treat or prevent adult onset Still's disorder. Activity of Th17 cells has been implicated in the development of graft-versus-host disease following allogeneic stem cell (e.g., bone marrow) transplantation (Fujiwara, et al., 2014). In some embodiments, the compounds provided herein may be administered to a patient in order to treat or prevent graft-versus-host disease, for example, following allogeneic stem cell (e.g., bone marrow) transplantation. Given the large body of evidence to date, it is likely that therapies reducing the expression of IL-17 or otherwise reducing its levels in circulation or target tissues (e.g., anti-IL17 monoclonal antibodies) could have broad applications in the treatment of autoimmune diseases and other inflammatory conditions. In some embodiments, the compounds provided herein may be administered to a patient in order to reduce the expression of IL-17 or its levels in circulation or target tissues (e.g., anti-IL17 monoclonal antibodies). In some embodiments, the compounds provided herein may be administered to a patient in order to treat autoimmune diseases or other inflammatory conditions.

Overproduction of IL-17 or elevated numbers of Th17 cells have been reported in patient studies or animal models of a large number of conditions, including autoimmune diseases, neurological disorders, cardiovascular diseases, cancer, psychiatric and neuropsychiatric disorders, acute and chronic inflammatory conditions, chronic pain syndromes, organ rejection or graft-versus-host disease, or asthma and other allergic conditions. In some embodiments, the compounds provided herein may be administered to a patient in order to treat or prevent one or more of these diseases or disorders.

Both the differentiation of Th17 cells and their production of IL-17 are regulated to a significant degree by the retinoid orphan receptor RORγt, a member of the nuclear hormone receptor family. Expression of RORγt is common to all types of Th17 cells. RORγ also regulates the production of IL-17 in other cell types, including T⁶ T cells, innate lymphoid cells, and lymphoid tissue inducer cells (Bronner et al., 2016). Inhibition of RORγt activity results in reduced expression of IL-17. In some embodiments, the compounds provided herein my be administered to a patient in order to inhibit RORγt activity.

Compounds and compositions provided and disclosed herein may be used to suppress IL-17 production in cultures of human T cells that are exposed to a mixture of cytokines known to induce differentiation into Th17 cells. In some embodiments, the ability to act as inverse agonists of RORγt is also demonstrated. Without wishing to be bound by any theory, it is believed that, for example, RORγt-independent mechanisms appear to contribute to the suppression of IL-17 production. Thus, the compounds and compositions provided herein may be used for inhibiting differentiation of T cells into Th17 cells, as well as inhibiting production of IL-17 by mature Th17 cells. In some of these embodiments, the net result is a reduction in IL-17 levels. In some embodiments, the compounds provided herein may be administered to a patient in order to suppress IL-17 production in one or more of the patient's tissues or organs.

### III. Pharmaceutical Formulations and Routes of Administration

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

$$HED ( mg / kg ) = Animal dose ( mg / kg ) \times ( Animal {K}_{m} / Human {K}_{m} )$$

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### IV. Combination Therapy

In addition to being used as a monotherapy, the compounds of the present invention may also find use in combination therapies. Effective combination therapy may be achieved with a single composition or pharmacological formulation that includes both agents, or with two distinct compositions or formulations, administered at the same time, wherein one composition includes a compound of this invention, and the other includes the second agent(s). Alternatively, the therapy may precede or follow the other agent treatment by intervals ranging from minutes to months.

Non-limiting examples of such combination therapy include combination of one or more compounds of the invention with another anti-inflammatory agent, a chemotherapeutic agent, radiation therapy, an antidepressant, an antipsychotic agent, an anticonvulsant, a mood stabilizer, an anti-infective agent, an antihypertensive agent, a cholesterol-lowering agent or other modulator of blood lipids, an agent for promoting weight loss, an antithrombotic agent, an agent for treating or preventing cardiovascular events such as myocardial infarction or stroke, an antidiabetic agent, an agent for reducing transplant rejection or graft-versus-host disease, an anti-arthritic agent, an analgesic agent, an anti-asthmatic agent or other treatment for respiratory diseases, or an agent for treatment or prevention of skin disorders. Compounds of the invention may be combined with agents designed to improve a patient's immune response to cancer, including (but not limited to) cancer vaccines. See Lu et al. (2011), which is incorporated herein by reference.

### V. Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “ [symbol] ” means a single bond, ‘ [symbol] ’ means a double bond, and “ [symbol] ” means triple bond. The symbol “ [symbol] ” represents an optional bond, which if present is either single or double. The symbol “ [symbol] ” represents a single bond or a double bond. Additionally, this symbol may be used to refer to an epoxidized double bond such as the group found in the compounds described in PCT/US2013/045975 (WO 2013/188818), the entire contents of which are hereby incorporated by reference. Thus, the formula

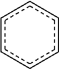

covers, for example,

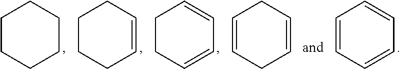

And it is understood that no one such ring atom forms part of more than one double bond. Furthermore, it is noted that the covalent bond symbol “ [symbol] ”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

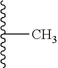

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

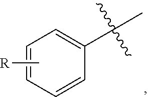

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

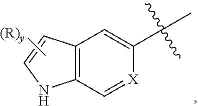

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “cycloalkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system.

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂-(methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen, alkyl, aryl, or heteroaryl. Non-limiting examples of alkylidene groups include: ═CH₂, =CH(CH₂CH₃), and ═C(CH₃)₂. An “alkane” refers to the class of compounds having the formula H-R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

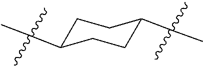 [SMILES: *C1CCC(*)CC1]

is a non-limiting example of cycloalkanediyl group. A “cycloalkane” refers to the class of compounds having the formula H-R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H-R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H-R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl, aryl, or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

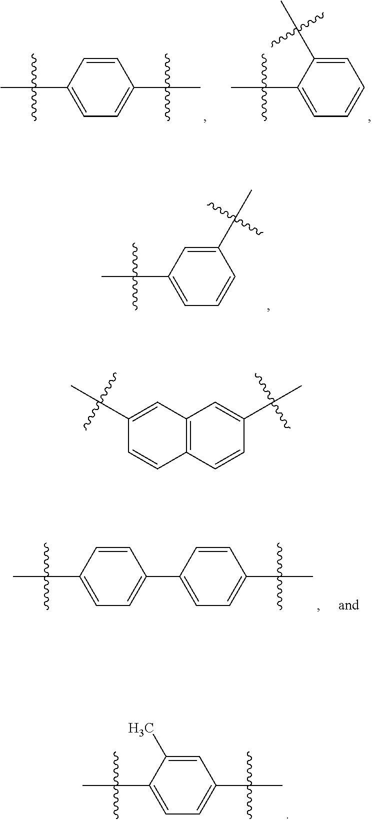

An “arene” refers to the class of compounds having the formula H-R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused. The term heteroaryl does not preclude the presence of one or more alkyl, cycloalkyl, heterocycloalkyl, aryl, or aralkyl groups (carbon number limitation permitting) attached to one or more ring atoms. Non-limiting examples of heteroaryl groups include benzthiazolyl, benzimidazolyl, furanyl, imidazolyl (Im), indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H-R, wherein R is heteroaryl. Pyridine and quinoline are non-limiting examples of heteroarenes.

The term “heteroarenediyl” refers to a divalent aromatic group, with two aromatic carbon atoms, two aromatic nitrogen atoms, or one aromatic carbon atom and one aromatic nitrogen atom as the two points of attachment, said atoms forming part of one or more aromatic ring structures, each with three to eight ring atoms, wherein at least one of the ring atoms of the aromatic ring structure(s) is nitrogen, oxygen or sulfur, and wherein the divalent group consists of no atoms other than carbon, hydrogen, aromatic nitrogen, aromatic oxygen and aromatic sulfur. If more than one ring is present, the rings are fused; however, the term heteroarenediyl does not preclude the presence of one or more alkyl or aryl groups (carbon number limitation permitting) attached to one or more ring atoms. Non-limiting examples of heteroarenediyl groups include:

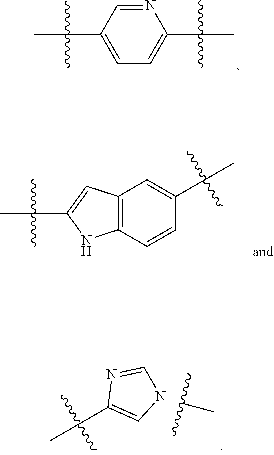

The term “heteroaralkyl” refers to the monovalent group -alkanediyl-heteroaryl, in which the terms alkanediyl and heteroaryl are each used in a manner consistent with the definitions provided above. Non-limiting examples are: pyridinylmethyl and 2-quinolinyl-ethyl.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl or cycloalkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “heterocycloalkanediyl” refers to a divalent cyclic group, with two carbon atoms, two nitrogen atoms, or one carbon atom and one nitrogen atom as the two points of attachment, said atoms forming part of one or more ring structure(s) wherein at least one of the ring atoms of the non-aromatic ring structure(s) is nitrogen, oxygen or sulfur, and wherein the divalent group consists of no atoms other than carbon, hydrogen, nitrogen, oxygen and sulfur. If more than one ring is present, the rings are fused. As used herein, the term heterocycloalkanediyl does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkanediyl groups include:

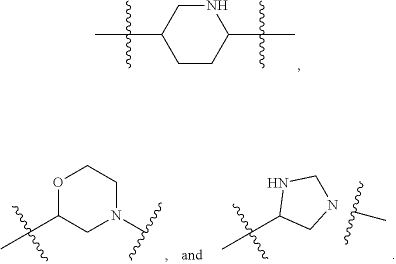

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The terms “dicycloalkylamino”, “dialkenylamino”, “dialkynylamino”, “diarylamino”, “diaralkylamino”, “diheteroarylamino”, “diheterocycloalkylamino”, and “dialkoxyamino”, refers to groups, defined as —NRR′, in which R and R′ are both cycloalkyl, alkenyl, alkynyl, aryl, aralkyl, heteroaryl, heterocycloalkyl, and alkoxy, respectively. Similarly, the term alkyl(cycloalkyl)amino refers to a group defined as —NRR′, in which R is alkyl and R′ is cycloalkyl. The term “amido” (acylamino), when used without the “substituted” modifier, refers to the group —NHR, in which R is acyl, as that term is defined above. A non-limiting example of an amido group is —NHC(O)CH₃.

The terms “alkylsulfonyl” refers to the group: —S(O)₂R, in which R is an alkyl, as that term is defined above. The terms “cycloalkylsulfonyl”, “alkenylsulfonyl”, “alkynylsulfonyl”, “arylsulfonyl”, “aralkylsulfonyl”, “heteroarylsulfonyl”, “heterocycloalkylsulfonyl”, and “alkoxysulfonyl” are defined in an analogous manner, wherein R is a cycloalkyl, alkenyl, alkynyl, aryl, aralkyl, heteroaryl, heterocycloalkyl, and alkoxy group, respectively, as those terms are defined above.

When a chemical group is used with the “substituted” modifier, one or more hydrogen atom has been replaced, independently at each instance, by —OH, —F, —Cl, —Br, —I, —NH₂, —NO₂, —CO₂H, —C(O)H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio. One example of compounds which are pharmaceutically acceptable include those compounds, materials, compositions, and/or dosage forms have been designated by the United States Food and Drug Administration (US FDA) as having a status of generally regarded as safe (GRAS).

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VI. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials & Methods

RORγ LBD-GAL4 Assay. The RORγ assay system was purchased from Indigo Biosciences (State College, PA, USA). The assay utilizes a human cell line engineered to provide high level expression of a hybrid form of the Human RAR-related Orphan Receptor Gamma (RORγ). The N-terminal DNA binding domain (DBD) of the native RORγ receptor was substituted with the yeast GAL4-DBD to generate the GAL4-RORγ hybrid nuclear receptor. The reporter cell line is transfected with a plasmid that encodes the beetle luciferase gene under the control of the GAL4 upstream activating sequence (UAS). The GAL4-RORγ hybrid is constitutively active providing measurable levels of basal Luciferase activity. To assess the RORγ inverse-agonist activity of the test compounds, reporter cells were plated in 96-well plates in triplicate and were treated with DMSO (vehicle) or different concentrations of test compounds at 37° C. with 5% CO₂ in a humidified atmosphere for 23 hours. After that, luciferin was added to the wells and luciferase activity was determined by measuring the luminescence signal using a BMG Pherastar microplate reader. Values from test compound samples were normalized to those from DMSO-treated samples.

Full-length RORgt activity in Jurkat Cells. RORγt transcriptional activity was assessed by transfecting human Jurkat T lymphocytes with each full-length human ROR isoform expression construct together with a 5x-RORE luciferase reporter construct and assaying luciferase activity. Expression plasmids consisting of human ROR isoform cDNA in a pReceiver-M02 vector were purchased from GeneCopoeia. Specific constructs included: RORA, transcript variant 1 (Catalog #EX-T3100-M02-B); RORB (Catalog #EX-Z5728-M02-B); and RORC, transcript variant 2 (Catalog #EX-T6988-M02-B). The pReceiver-M02 empty vector (Catalog #EX-NEG-M02) was used as control. p5xRORE_sluc3.3 luciferase reporter plasmid was synthesized at Blue Heron Biotech, LLC, by inserting 5 copies of the ROR response element sequence (5′-AAAGTAGGTCA-3′ (SEQ. NO. 3) in XhoI/HindIII restriction sites of the pNL3.3[secNLuc/minP] vector (Promega).

Jurkat cells (Human peripheral blood T lymphocytes) were cultured in RPMI 1640 medium (Life Technologies) supplemented with 10% delipidated FBS (Gemini Bio-Products) and 1% penicillin-streptomycin in a humidified atmosphere at 37° C. with 5% CO₂. Cells were seeded in a 75 cm² flask at a density of 0.3×106 cells/mL of growth media. After 18 h, cells were re-seeded into 25 cm² flasks at a density of 0.6×106 cells/mL. Cells were transfected using Lipofectamine LTX with Plus reagent according to the manufacturer's protocol, with 0.8 μg p5xRORE_sluc3.3 luciferase reporter plasmid and 0.8 g of pReceiverM02 control, pRORA, pRORB, or pRORC plasmids (1.6 g plasmid DNA in total) in a total volume of 250 μL Opti-MEM per mL of cell suspension. 6 h after transfection, cells were diluted with growth medium and plated in 96-well plates at a density of 5×104 cells per well in 190 μL medium. Immediately following plating, transfected cells were treated for 18 h with vehicle (DMSO) or compounds at concentrations ranging from 0 to 500 nM in a two-fold dilution series. Final DMSO concentration in each well was 0.1%. Three replicate wells were tested for each treatment condition.

NanoLuc luciferase activity was measured using the Nano-Glo Luciferase Assay System (Promega) according to the manufacturer's protocol. Briefly, after shaking the 96-well plates on an orbital shaker at 100 RPM for 2 min and centrifuging at 250×g RPM at room temperature for 5 min, 60 μL of media was removed from each well and transferred to the corresponding well of a 96-well V-bottom plate. Ten (10) μL of this media was transferred to duplicate wells of a 384-well Small Volume HiBase plate, resulting in a total of six luciferase activity measurements for each concentration of the test compound. NanoLuc assay reagent (10 μL) was added to each well. After shaking the 384-well plate at 500 RPM for 2 min and incubating at room temperature for an additional 3 min, the luminescence signal was measured on a PHERAstar FS microplate reader (BMG Labtech).

After removing the aliquot of medium for the luciferase assay, 14 μL (10% of remaining volume) WST-1 reagent was added to each well of the 96-well plate and incubated at 37° C. with 5% CO₂ for 30 min. Molecular Devices SpectraMax M2e plate reader was used to measure absorbance of the formazan dye at 450 nm and background at 650 nm.

IL-17 Release from Th17-polarized Primary Human T-Cells. Primary human cryopreserved CD4+ T Cells (Lonza) were thawed according to the manufacturer's recommendations and plated either in Lymphocyte Growth Medium-3 (LGM-3) or X-VIVO 20 media (Lonza) in 96-well tissue culture plates at a density of ˜2×10⁵ cells per well, and allowed to recover for approximately 4 hours at 37° C. with 5% CO₂ in a humidified atmosphere. After the recovery step, DMSO (vehicle) or test compound at doses ranging from 2 to 500 nM or 4 to 1000 nM in a three-fold dilution series was added to the cells. Three replicate wells were tested for each treatment condition. Final DMSO concentration in each well was 0.1%. Immediately after treatment, CD4+ T cells were activated by adding Dynabeads Human T-Activator CD3/CD28 (Life Technologies; bead-to-cell ratio of 1:2.5) and differentiated into Th17 cells by adding a mixture of the following cytokines: transforming growth factor-β (TGF-β, 5 ng/mL), IL-6 (20 ng/mL), IL-23 (20 ng/mL), and IL-13 (10 ng/mL). Undifferentiated control cells received only cytokine IL-2 (50 ng/mL). All human recombinant cytokines were purchased from R&D Systems. Following a 45-hour incubation at 37° C. with 5% CO₂ in a humidified atmosphere, the plates were centrifuged for 3 minutes at 250×g, and half of the supernatant was transferred to a new plate to be used in the IL-17A assay. The concentration of IL-17A in the supernatant was measured using the Homogeneous Time-Resolved Fluorescence (HTRF) assay (Cisbio Bioassays) according to the manufacturer's protocol. The assay was performed at room temperature in low volume, solid white 384-well plates (Greiner Bio-One). Samples and standards (serially-diluted human recombinant IL-17A (0 to 5,000 μg/mL concentration range; Cisbio Bioassays) were incubated with the anti-human IL-17A antibody conjugates (the HTRF donor and acceptor pair) for 16 hours and fluorescence was measured using a Pherastar FS microplate reader (BMG Labtech) in the HTRF mode (excitation at 337 nm and emission at 665 nm and 620 nm). IL-17A levels were assessed in duplicate aliquots of supernatant from each well resulting in a total of six readings per test condition. The 665 nm/620 nm signal ratio was calculated and the concentration of IL-17A in each sample was determined by interpolation from the standard curve. The amount of IL-17A from test compound treated samples were normalized to that of the vehicle-treated samples, set to 100%. Data were analyzed using GraphPad Prism (GraphPad Software, La Jolla California USA). Concentrations of test compound were transformed by taking the logarithm of each concentration used. The IC₅₀ values for compound-mediated reduction in IL-17A levels and cell viability were determined by non-linear regression analysis using the log(inhibitor) vs. normalized response with variable slope equation.

Naïve T cell differentiation and intracellular staining. Naïve CD4+ T cells were isolated from cryopreserved human PBMCs (ZenBio) using an EasySep human naïve CD4+ T cell isolation kit (StemCell Technologies). The purity of cells (CD4+CD45RA+≥95%) was verified by flow cytometry. Naïve cells (2×10⁵ cells/well) were cultured in X-VIVO 20 media (Lonza) in 24-well tissue culture plates pre-coated with anti-CD3 antibody (1 μg/mL). Immediately following plating (day 0), cells were treated with vehicle (DMSO) or compound 14 at indicated concentrations. Cells were activated with anti-CD28 antibody (1 μg/mL) and cultured for 6 days under either neutral (Th0) or Th17-polarizing (Th17) conditions (TGF-0 (5 ng/mL), IL-6 (20 ng/mL), IL-10 (10 ng/mL), IL-23 (20 ng/mL), anti-IL-4 antibody (1 μg/mL), and anti-IFN-7 antibody (1 μg/mL)). All cytokines were from R&D Systems, and all antibodies were from Biolegend. On day 5, cells were resuspended in the same media and reactivated by re-plating in new 24-well plates coated with anti-CD3 antibody (1 μg/mL). On day 6, cells were restimulated with Cell Stimulation Cocktail (eBioscience) in the presence of BD GolgiStop™ Protein Transport Inhibitor for the last 5 h, stained with Fixable Viability Dye eFluor 520 followed by fixation with BD Fixation buffer, permeabilized with BD Perm/Wash buffer and stained with AF647 anti-human IL-17A antibody. Data were acquired using a BD Accuri™ C6 or C6 Plus flow cytometer (BD Biosciences) and analyzed using FlowJo Software (Tree Star Inc). Lymphocytes were gated based on forward vs. side scatter (FSC vs. SSC) plot. Dead cells were subsequently excluded from analysis based on Fixable Viability Dye eFluor520 staining. At least 20,000 events per sample were analyzed.

RNA extraction, quantitative RT-PCR. For qPCR analysis, human naïve T-cells were differentiated under Th-17 condition for six days as described under Naïve T cell differentiation and intracellular staining protocol. Total RNA was extracted using RNeasy kits including the optional DNaseI digestion (Qiagen). Following cDNA synthesis, Real Time quantitative PCR was performed on a 7900HT Real Time PCR System (Applied Biosystems).

### Example 1: Activity of RORγ/RORγt Inhibitors

Screening a series of tricyclic compounds in a RORγt-LBD-GAL4 reporter assay, identified the tricyclic pyrimidine 1 with inhibitory activity (IC₅₀=608 nM) (Table 1).

Sequential introduction of C-12 phenyl and 4-pyridyl groups afforded compound 3 a 9-fold increase in activity relative to 1, with an IC₅₀ of 68 nM. Compound 3 was characterized further in a phenotypic assay where the amount of IL-17A produced by Th17 polarized human CD4+ T cells was measured. Consistent with the essential role that RORγt plays in controlling IL-17A expression, potent suppression of IL-17A secretion from human CD4+ T-cells treated with compound 3 (IC₅₀=76 nM) was observed. The combination of an alkyl substituted C-12 pyridyl ring with a fluoro phenyl C-14 substituent, afforded compounds 12-16 which were quite potent in the RORγt GAL4 transcriptional assay as well as in the L-17 phenotypic assay (Table 1). Further modification of the A ring, B ring, C4 and C10 was carried out to determine the effects of these changes on the potency in the RORγt GAL4 transcriptional assay as well as in the IL-17 phenotypic assay (Tables 2 and 3).

TABLE 1
Activity of Compound with Modifications at
the C12 and C14 Positions of the Pyrimidine Ring.

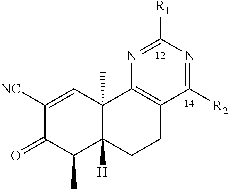

| | | | CD4+ | |
|---|---|---|---|---|
| | | | T-cell | GAL4 |
| | | | IL17 | Luc |
| | | | IC₅₀ | IC₅₀ |
| Compd | R₁ | R₂ | (μM) | (μM) |
| 1 | H | Phenyl | 0.071 | 0.613 |
| 2 | Phenyl | Phenyl | 0.030 | 0.162 |
| 3 | 4′-Pyridyl | Phenyl | 0.076 | 0.067 |
| 4 | 4′-Pyridyl | 4′-Pyridyl | 0.128 | 0.254 |
| 5 | 4′-Pyridyl | 3′-Pyridyl | 0.154 | 0.212 |
| 6 | 4′-Pyridyl | 4′-hydroxy- | 0.201 | 0.203 |
| | | methylphenyl | | |
| 7 | 4′-Pyridyl | Methoxy | 0.461 | 0.163 |
| 8 | 4′-Pyridyl | Benzyl | 0.132 | 0.160 |
| 9 | Morpholine | 2′-F-Phenyl | 0.097 | 0.136 |
| 10 | N′-Acetyl piperazine | 2′-F-Phenyl | 0.104 | 0.141 |
| 11 | N′-Methyl piperazine | 2′-F-Phenyl | 0.236 | 0.314 |
| 12 | 3′-Methyl-4′-Pyridyl | 2′-F-Phenyl | 0.048 | 0.060 |
| 13 | 2′-Methyl-4′-Pyridyl | 2′-F-Phenyl | 0.046 | 0.060 |
| 14 | 3′-Methyl-4′-Pyridyl | 4′-F-Phenyl | 0.040 | 0.058 |
| 15 | 3′-Fluoromethyl- | 2′-F-Phenyl | 0.035 | 0.074 |
| | 4′-Pyridyl | | | |
| 16 | 3′-Cyclopropyl- | 2′-F-Phenyl | 0.031 | 0.097 |
| | 4′-Pyridyl | | | |

TABLE 2
Activity of Compound with Modifications at C4, C10 and the B-ring

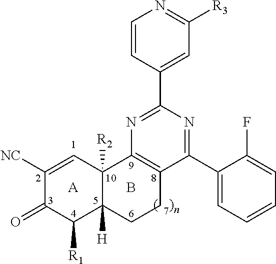

| | | | | | GAL4 | |
|---|---|---|---|---|---|---|
| | | | | CD4+ | Luciferase | |
| | | | | T-Cells IL17 | Reporter | |
| Compd | R₁ | R₂ | B-ring (n) | IC₅₀ (μM) | IC₅₀ (μM) | R₃ |
| 16 | CH₃ | CH₃ | cyclohexyl | 0.031 | 0.097 | 3′-Cyclopropyl |
| | | | (n = 1) | | | |
| 17 | CH₃ | Phenyl | cyclohexyl | 0.046 | 0.475 | 3′-Cyclopropyl |
| | | | (n = 1) | | | |
| 18 | CH₃ | n-Propyl | cyclohexyl | 0.026 | 0.140 | 3′-Cyclopropyl |
| | | | (n = 1) | | | |
| 19 | H | iso-Pentyl | cyclohexyl | 0.071 | 0.402 | 3′-Fluoromethyl |
| | | | (n = 1) | | | |
| 20 | Allyl | CH₃ | cyclohexyl | 0.020 | 0.072 | 3′-Cyclopropyl |
| | | | (n = 1) | | | |
| 21 | n-Propyl | CH₃ | cyclohexyl | 0.012 | 0.082 | 3′-Cyclopropyl |
| | | | (n = 1) | | | |
| 22 | n-Propyl | CH₃ | cyclohexyl | 0.012 | 0.092 | 3′-CF₃ |
| | | | (n = 1) | | | |
| 23 | n-Propyl | CH₃ | cyclohexyl | 0.017 | 0.051 | 3′-CH₂F |
| | | | (n = 1) | | | |
| 24 | CH₃ | CH₃ | cyclopentyl | 0.140 | 0.334 | 3′-CH₂F |
| | | | (n = 0) | | | |
| 25 | CH₃ | CH₃ | cyclopentyl | 0.117 | 0.524 | 3′-Cyclopropyl |
| | | | (n = 0) | | | |
| 26 | CH₃ | CH₃ | cycloheptyl | 0.076 | 0.068 | 3′-CH₃ |
| | | | (n = 2) | | | |

TABLE 3
Activity of Compound with Modifications at the Michael Acceptor A-ring

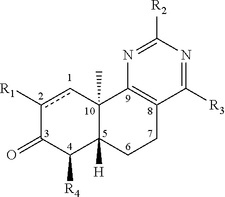

| | | | CD4+ | GAL4 Luc | | | |
|---|---|---|---|---|---|---|---|
| | | | T-cells IL17 | Reporter | | | |
| Compd | R₁ | C1, C2 bond | IC₅₀ (μM) | IC₅₀ (μM) | R₂ | R₃ | R₄ |
| 3 | CN | unsaturated | 0.076 | 0.068 | 4′-Pyridyl | Phenyl | Methyl |
| 27 | CONH₂ | unsaturated | 0.181 | 0.337 | 4′-Pyridyl | Phenyl | Methyl |
| 14 | CN | unsaturated | 0.04 | 0.058 | 3′-Methyl-4′-Pyridyl | p-Fluorophenyl | Methyl |
| 28 | CONH₂ | unsaturated | 0.107 | 0.295 | 3′-Methyl-4′-Pyridyl | p-Fluorophenyl | Methyl |
| 21 | CN | unsaturated | 0.012 | 0.082 | 3′-cyclopropyl-4′-Pyridyl | o-Fluorophenyl | n-propyl |
| 29 | CONH₂ | unsaturated | 0.036 | 0.293 | 3′-cyclopropyl-4′-Pyridyl | o-Fluorophenyl | n-propyl |
| 30 | H | unsaturated | 0.328 | 0.310 | 4′-Pyridyl | Phenyl | Methyl |
| 31 | CN | saturated | 0.476 | 0.643 | 4′-Pyridyl | Phenyl | Methyl |
| 32 | CONH₂ | saturated | >2 | 5.39 | 4′-Pyridyl | Phenyl | Methyl |
| 33 | CN | saturated | 0.154 | 1.24 | 3′-cyclopropyl-4′-Pyridyl | o-Fluorophenyl | n-propyl |

The relationship between biological activity and the A-ring Michael acceptor is consistent with a covalent drug:protein interaction, most likely involving one of the 8 free cysteines in the RORγ/RORγt LBD. While this mechanism of action (MOA) has been exploited successfully against other protein classes, most notably with kinases, it is rare in the nuclear receptor (NR) field, especially in the ROR family of NRs. The chemical biology literature of NRs provides few insights on how to identify the specific cysteine in the LBD of RORγ/RORγt central to the inverse agonism observed. PPARγ was the lone exception, where several endogenous fatty acid metabolite ligands (e.g. PGJ₂), have been shown to bind in the orthosteric site, ligate Cysteine 285 on alpha helix H3, and regulate the NR's transcriptional activity. Interestingly, Cysteine 320 on alpha helix H3 of RORγt occupies precisely the same location relative to the RORγt orthosteric site, as Cysteine 285 does on PPARγ. With this in mind, full-length human RORγt constructs were generated where Cysteine 320 (Cysteine 299 in RORγt) was mutated to Alanine as well as Serine, and the activity of compounds 14 and 21 in full-length RORγt RORE-luciferase reporter assays in Jurkat cells was assessed using these mutants. Compounds 14 and 21 were found to be refractory to both mutations, displaying similar activity against the wild-type and mutant RORγt proteins (Table 4). Supportive of these results, compounds 14 and 21 were found not to compete with cholesterol sulfate, a known orthosteric RORγt ligand, in competitive binding experiments using the RORγt LBD.

TABLE 4
Effect of Cysteine Mutations in Human RORγ
on Inhibitory Activity of Selected Compounds

RORγt IC₅₀ (nM)

| RORγ (RORγt)^{a} | Compound 14 | Compound 21 |
|---|---|---|
| Wild Type | 35.7 +/− 13.1 | 39.5 +/− 9.5 |
| C320S (C299S) | 36.7 +/− 13.4 | 58.3 +/− 20.9 |
| C320A (C299A) | 43.7 +/− 11.8 | 64.1 +/− 24.0 |
| C476S (C455S) | ND | ND |
| C476A (C455A) | ND | ND |
| C393S (C372S) | 60.4 +/− 25.2 | 48.7 +/− 20.0 |
| C285S (C264S) | 37.6 +/− 17.2 | 28.1 +/− 9.7 |
| C455S (C434S) | 37.5 +/− 17.9 | 52.6 +/− 18.5 |
| C278S (C257S) | 41.3 +/− 14.9 | 46.5 +/− 13.8 |
| C366S (C345S) | 39.0 +/− 7.6 | 50.3 +/− 12.3 |
| C345S (C324S) | 33.9 +/− 14.8 | 37.4 +/− 11.3 |

ND—IC₅₀ value could not be determined because RORγt was not inhibited at concentrations that did not affect cellular viability
^{a}Residue numbering is according to full-length human RORγ with human RORγt numbering shown in brackets

^{a}Residue numbering is according to full-length human RORγ with human RORγt numbering shown in brackets

Next, expression plasmids serially mutating the seven remaining free cysteine residues on RORγ to serine and/or alanine were generated. In subsequent reporter gene assays, only mutation of Cysteine 476 affected the ability of compounds 14 and 21 to inhibit RORγ (Table 4). In addition, the binding of compound 21 to Cysteine 476 was confirmed by hydrogen/deuterium exchange (HDX) mass spectrometry (MS).

Cysteine 476 resides on alpha helix H11 of RORγ and coincidentally is on the periphery of the allosteric site previously described by Merck and Eindhoven. In binding experiments, compound 21 and MRL-871, a known allosteric modulator of RORγ, were found to be mutually competitive. Moreover, no complex was observed in which MRL-871 and compound 21 were simultaneously bound to RORγt. However, the binding of MRL-871 was not affected by the presence of the C476S mutation. In addition, MRL-871 displayed similar activity against the wild-type and C476S mutant RORγt protein. Taken together these data support a MOA where compound 12 binds to an allosteric site through a specific covalent interaction with Cysteine 476, thereby inhibiting the transcriptional activity of RORγ/RORγt.

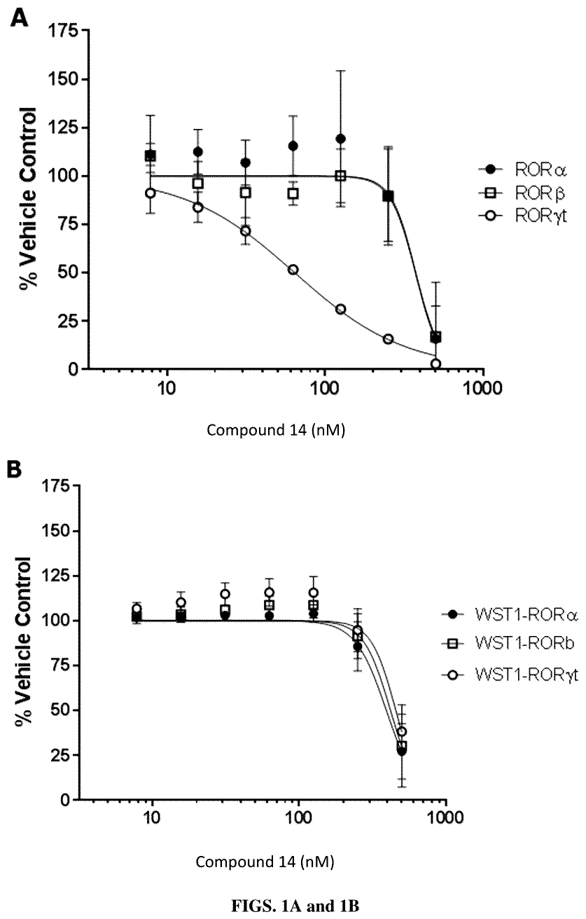

Cysteine 476 is strictly conserved among all three ROR isoforms: alpha, beta, and gamma. Nonetheless, it is rarely found in the remaining 45 human NRs, occurring only in the orphan receptors nerve growth factor1B (NGF1B), testicular receptor2 (NR2C2), and nuclear receptor related1 (NR4A2) receptors. The inhibitory activity of compound 14 was evaluated against a panel of seventeen human NRs from the NR1 subfamily and found no appreciable activity at concentrations that did not affect cell viability. Furthermore, compounds 14 and 21 showed good ROR isoform selectivity, inhibiting RORγ/RORγt activity without measurable effects on RORα or RORβ activity at concentrations that did not affect cell viability (see FIGS. 1A-1B for data using compound 14).

Towards the goal of developing a predictive model consistent with the observed potency, selectivity and MOA of this drug class, molecular dynamics (MD) simulations were utilized. A covalent docking protocol was developed within MOE and applied to compound 21 using the MRL-871/RORγt protein crystal structure.

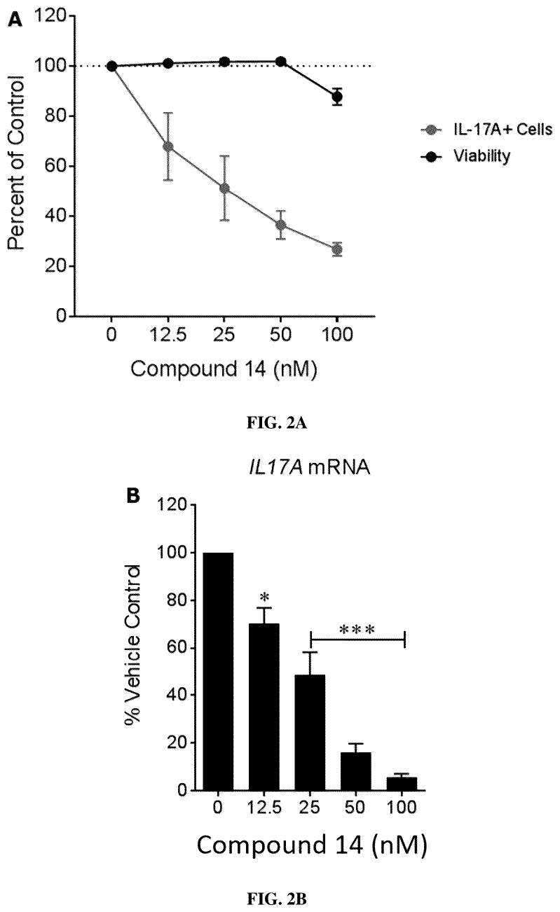

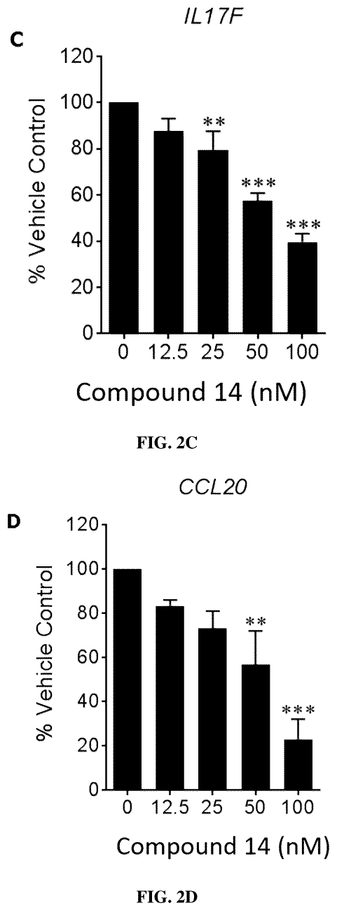

The biological activity of compound 14 was further characterized by assessing its ability to inhibit Th17 differentiation of human naïve CD4+ T cells cultured in vitro under Th17-polarizing conditions. Compound 14 potently inhibited Th17 differentiation, as measured by the percentage of IL-17A-expressing cells, with an average IC₅₀ value of 30 nM (FIG. 2A). Compound 14 also significantly reduced the expression of key Th17 signature genes, including IL17A, IL17F, and CCL20 (FIGS. 2B-6D). Under the Th17 differentiating protocol used for these experiments, compound 14 reduced cell viability by ˜10-15% at the highest concentration (FIG. 2A).

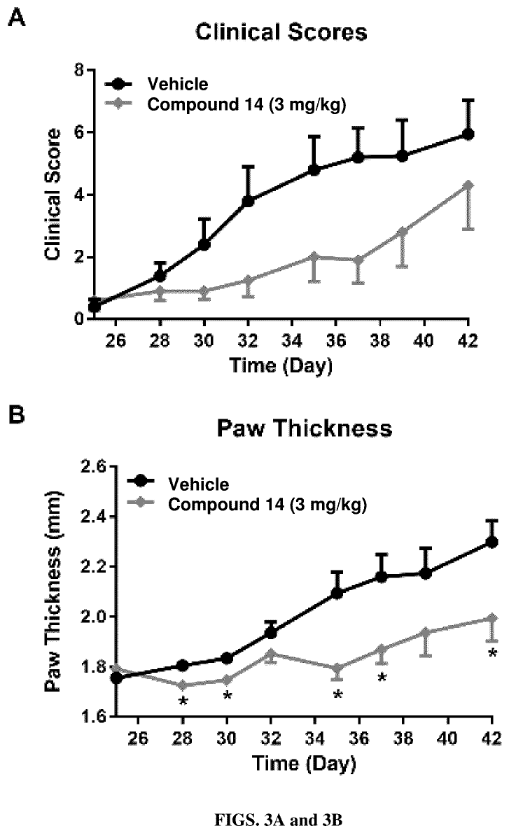

Compound 14 was evaluated for oral bioavailability, and consistent with its physicochemical properties, compound 14 easily achieves therapeutic systemic exposures after a single oral dosing to mice. Compound 14 was then evaluated for pharmacological activity in the collagen-induced rheumatoid arthritis (CIA) mouse model, as RORγt has been shown to be important in the pathophysiology of this model (Park, 2014) and in patients with rheumatoid arthritis (Paradowska-Gorycka, 2016). The study was conducted and progression of disease severity evaluated as previously described (Scales, 2016). Compound 14 significantly decreased both the clinical scores and paw thickness measurements, starting as early as one week after starting treatment and continuing until termination of the study on Day 42 (FIGS. 3A-3B). Collectively, these data provide in vivo proof of concept for this series of compounds.

### Example 2: Compound Synthesis and Characterization

### General Information

Unless otherwise stated, commercially reagents were used as received, and all reactions were run under nitrogen atmosphere. All solvents were of HPLC or ACS grade. Nuclear magnetic resonance (NMR) spectra were recorded on a Varian Inova-400 spectrometer at operating frequencies of 400 MHz (¹H NMR). Chemical shifts (δ) are given in ppm relative to residual solvent (usually chloroform δ 7.26 ppm for ¹H NMR), and coupling constants (J) in Hz. Multiplicity is tabulated as s for singlet, d for doublet, t for triplet, q for quadruplet, and m for multiplet. Mass spectra were recorded on Agilent 6120 mass spectrometer.

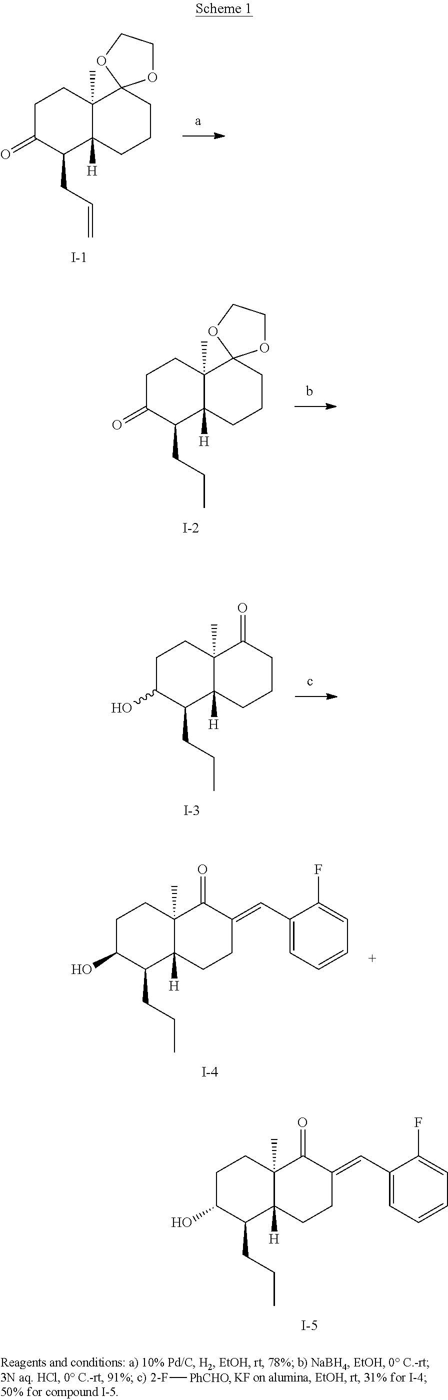

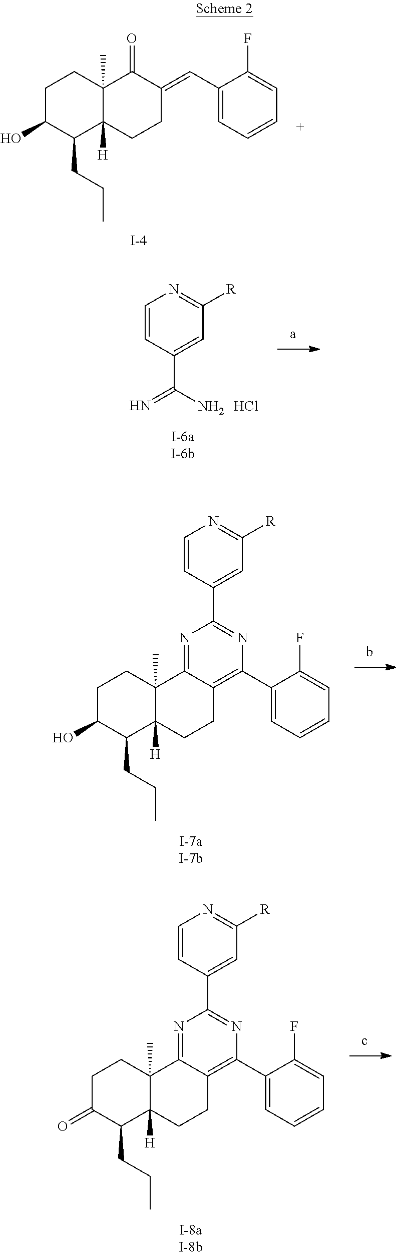

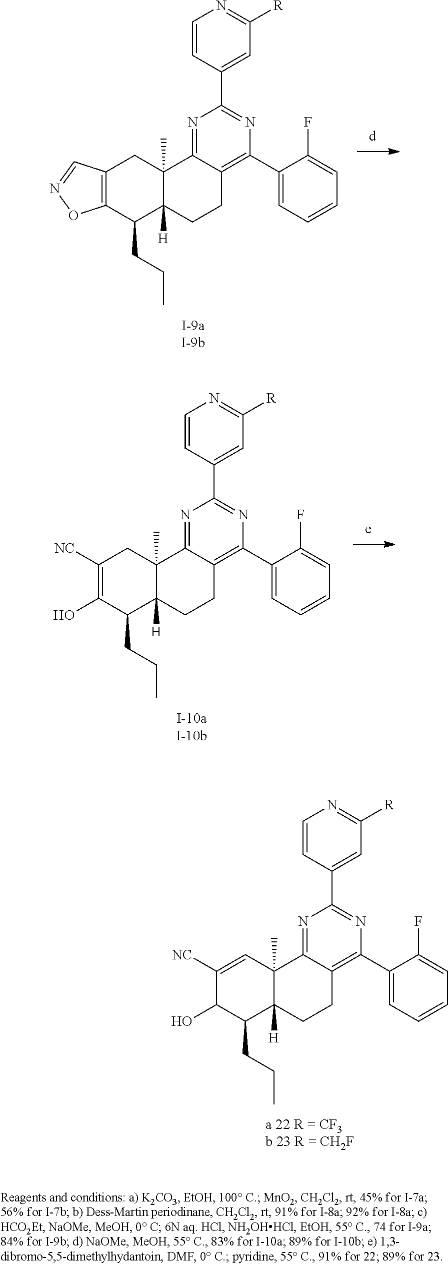

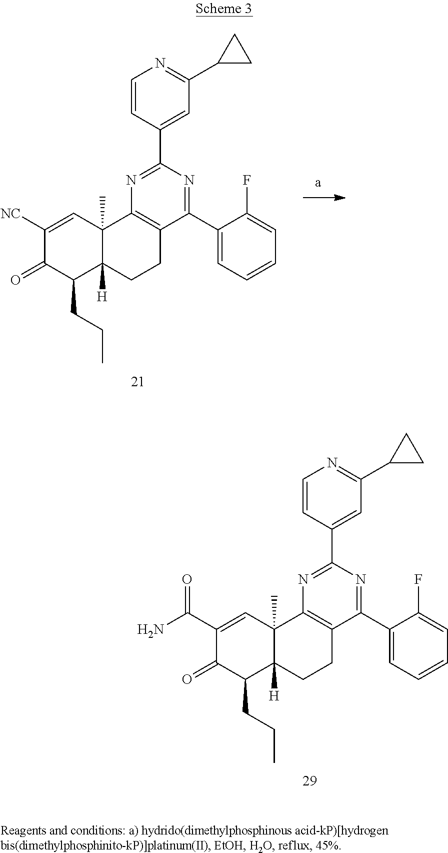

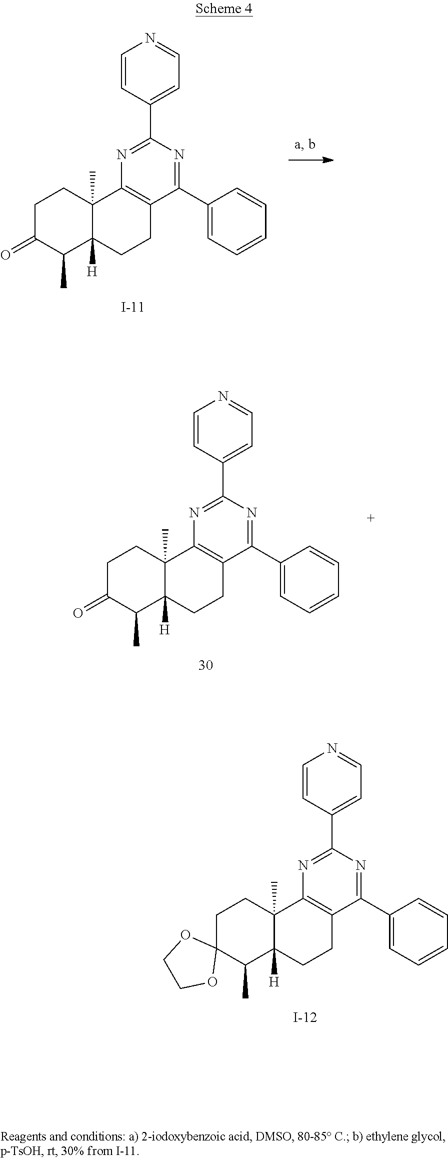

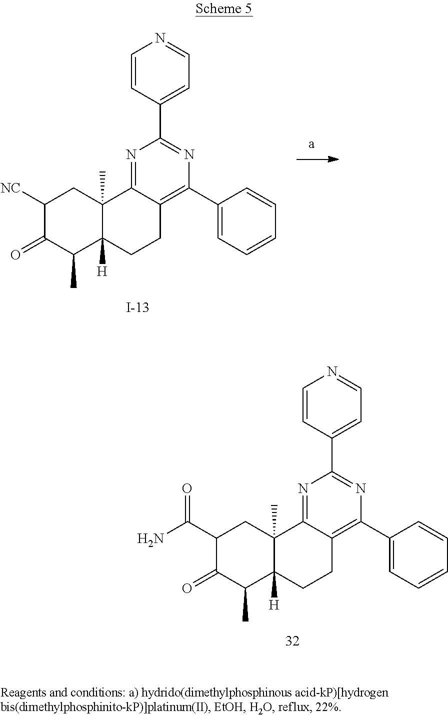

Compound I-2: To a solution of compound I-1 (20 g, 71.87 mmol) in EtOH (200 mL) was added 10% wet Pd/C (2 g). The mixture was stirred at 20° C. for 16 h under hydrogen (15 Psi) and filtered. The filtrate was concentrated to give compound I-2 (15.8 g, 78% yield) as a colorless oil. m/ [symbol] =267 (M+1).
Compound I-3: To a solution of compound I-2 (5.022 g, 18.85 mmol) in EtOH (50 mL) was added NaBH₄ (357 mg, 9.38 mmol) at 0° C. in 1 portion. After the mixture was stirred at 0° C. for 3 h, additional amount of NaBH₄ (100 mg, 2.62 mmol) was added. The mixture was stirred at ambient temperature for 30 min. TLC indicated the starting material was completely consumed. The mixture was cooled to 0° C. 3 N aq. HCl (80 mL, 240 mmol) was added. The mixture was stirred at room temperature for 20 h and concentrated. The residue was extracted with EtOAc (2×30 mL). The combined organic extracts were dried with Na₂SO₄, filtered, and concentrated. The residue was purified by column chromatography (silica gel, eluting with 0% to 40% EtOAc in hexanes) to give compound I-3 (3.850 g, 91% yield) as viscous oil. Compound I-3 is 1.6/1 mixture of diastereomers (determined by ¹H NMR).
Compound I-4 and I-5: To a solution of compound I-3 (1.011 g, 4.51 mmol) in EtOH (15 mL) were added 2-fluorobenzaldehyde (697 mg, 5.62 mmol) and potassium fluoride on alumina (5.5 mmol/g, 1.23 g, 6.765 mmol) sequentially at room temperature. The mixture was stirred at room temperature under N₂ for 20 h, diluted with EtOAc (30 mL), and filtered through a pad of Celite®. The filter cake was washed with EtOAc. The filtrate was concentrated, and the residue was partitioned between EtOAc (50 mL) and water (20 mL). The aqueous phase was extracted with EtOAc (30 mL). The combined organic extracts were dried with Na₂SO₄ and concentrated. The residue was purified by column chromatography (silica gel, eluting with 0 to 40% EtOAc in hexanes) to give compound I-4 (459 mg, 31% yield) and compound I-5 (743 mg, 50% yield) as white solids. Compound I-4: m/z=331 (M+1); Compound I-5: m/ [symbol] =331 (M+1).
Compound I-7a: A mixture of compound I-4 (152 mg, 0.46 mmol), compound I-6a (156 mg, 0.69 mmol) and K₂CO₃ (191 mg, 1.38 mmol) in EtOH (4 mL) was heated in a sealed vial at 100° C. for 48 h. After cooled to room temperature, the mixture was diluted with EtOAc (20 mL) and washed with water (3×15 mL) and brine (15 mL). The organic extract was dried with Na₂SO₄, filtered, and concentrated. The crude product was dissolved in CH₂Cl₂ (4 mL), and treated with MnO₂ (88%, 0.36 g, 3.65 mmol) at room temperature. The mixture was stirred at room temperature for 20 h and purified by column chromatography (silica gel, eluting with 0-50% EtOAc in hexanes) to give compound I-7a (103 mg, 45% yield) as a light yellow solid. m/z=500 (M+1).
Compound I-7b: Following the same procedure as described for the synthesis of compound I-7a, compound I-7b (light yellow solid; 119 mg, 56% yield) was synthesized from compound I-4 (152 mg, 0.46 mmol) and compound I-6b (131 mg, 0.69 mmol). m/z=464 (M+1).
Compound I-8a: Compound I-7a (96.0 mg, 0.19 mmol) and Dess-Martin periodinane (163 mg, 0.38 mmol) in CH₂Cl₂ (2 mL) was stirred at room temperature for 3 h. The mixture was treated with 10% aq. Na₂SO₃ (5 mL) and sat. aq. NaHCO₃ (5 mL), stirred at room temperature for 10 min, and extracted with t-butyl methyl ether (2×15 mL). The combined organic extracts were washed with sat. aq. NaHCO₃, dried with Na₂SO₄, filtered, and concentrated. The residue was purified by column chromatography (silica gel, eluting with 0-35% EtOAc in hexanes) to give compound I-8a (87 mg, 91% yield) as a white solid. m/ [symbol] =498 (M+1).
Compound I-8b: Following the same procedure as described for the synthesis of compound I-8a, compound I-8b (white solid; 105 mg, 92% yield) was synthesized from compound I-7b (115 mg, 0.25 mmol). m/z=462 (M+1).
Compound I-9a: To a mixture of compound I-8a (85 mg, 0.17 mmol) in ethyl formate (0.42 mL, 5.1 mmol) at 0° C. under N₂ was added sodium methoxide (4.37 M in MeOH, 0.39 mL, 1.7 mmol). The mixture was stirred at ambient temperature for 2 h and then cooled to 0° C. 6 N aq. HCl (0.28 mL, 1.7 mmol), EtOH (1.7 mL), and hydroxylamine hydrochloride (22 mg, 0.32 mmol) were added sequentially. The mixture was heated at 55° C. for 16 h, and then cooled to room temperature. The mixture was diluted with sat. aq. NaHCO₃ (5 mL) and extracted with EtOAc (20 mL). The organic extract was washed with water (10 mL), dried with Na₂SO₄, filtered, and concentrated. The residue was purified by column chromatography (silica gel, eluting with 0 to 40% EtOAc in hexanes) to give compound I-9a (66 mg, 74% yield) as a light yellow solid. m/ [symbol] =523 (M+1).
Compound I-9b: Following the same procedure as described for the synthesis of compound I-9a, compound I-9b (white solid; 91 mg, 84% yield) was synthesized from compound I-8b (103 mg, 0.22 mmol). m/ [symbol] =487 (M+1).
Compound I-10a: To a solution of compound I-9a (63 mg, 0.12 mmol) in MeOH (1.2 mL) at room temperature under N₂ was added sodium methoxide (4.37 M, 55 μL, 0.24 mmol). The mixture was stirred at 55° C. for 1.5 h, cooled to room temperature, treated with 10% aq. NaH₂PO₄ (5 mL), and extracted with EtOAc (30 mL). The organic extract was washed with water (10 mL), dried with MgSO₄, filtered, and concentrated. The residue was purified by column chromatography (silica gel, eluting with 0 to 50% EtOAc in hexanes) to give compound I-10a (52 mg, 83% yield) as a white solid. m/ [symbol] =523 (M+1).
Compound I-10b: Following the same procedure as described for the synthesis of compound I-10a, compound I-10b (white solid; 77 mg, 89% yield) was synthesized from compound I-9b (87 mg, 0.18 mmol). m/ [symbol] =487 (M+1).
Compound 22: Compound I-10a (52 mg, 0.10 mmol) in DMF (0.36 mL) was cooled to 0° C. under N₂. 1,3-dibromo-5,5-dimethylhydantoin (14 mg, 0.050 mmol) in DMF (0.14 mL) was added. The solution was stirred at 0° C. for 1 h. Pyridine (24 μL, 0.30 mmol) was added. The mixture was heated at 55° C. for 4 h, cooled to room temperature, diluted with EtOAc (20 mL), and washed with water (3×15 mL). The organic extract was dried with Na₂SO₄, filtered, and concentrated. The residue was purified by column chromatography (silica gel, eluting 0 to 50% EtOAc in hexanes) to give compound 22 (47 mg, 91% yield) as an off-white solid. m/ [symbol] =521 (M+1); ¹H NMR (400 MHz, CDCl₃) δ 8.94 (s, 1H), 8.91 (d, J=5.0 Hz, 1H), 8.70 (dd, J=1.5, 0.8 Hz, 1H), 8.57 (dd, J=5.1, 1.5 Hz, 1H), 7.54 (m, 1H), 7.45 (td, J=7.4, 1.8 Hz, 1H), 7.35 (td, J=7.5, 1.1 Hz, 1H), 7.23 (m, 1H), 2.93-2.77 (m, 2H), 2.61 (ddd, J=13.1, 5.0, 3.2 Hz, 1H), 2.46 (td, J=12.8, 2.5 Hz, 1H), 2.14-2.06 (m, 2H), 1.81 (m, 1H), 1.69 (m, 1H), 1.55 (s, 3H), 1.35 (m, 1H), 1.22 (m, 1H), 0.93 (t, J=7.3 Hz, 3H).
Compound 23: Following the same procedure as described for the synthesis of compound 22, compound 23 (off-white solid; 68 mg, 89% yield) was synthesized from compound I-10b (77 mg, 0.16 mmol). m/ [symbol] =485 (M+1); ¹H NMR (400 MHz, CDCl₃) δ 8.98 (s, 1H), 8.76 (d, J=5.2 Hz, 1H), 8.48 (s, 1H), 8.31 (ddd, J=5.1, 1.7, 0.8 Hz, 1H), 7.52 (m, 1H), 7.45 (td, J=7.4, 1.8 Hz, 1H), 7.34 (td, J=7.5, 1.1 Hz, 1H), 7.22 (ddd, J=9.7, 8.5, 1.1 Hz, 1H), 5.60 (d, J=46.9 Hz, 2H), 2.93-2.73 (m, 2H), 2.60 (ddd, J=13.1, 5.0, 3.2 Hz, 1H), 2.46 (td, J=12.8, 2.5 Hz, 1H), 2.15-2.05 (m, 2H), 1.82 (m, 1H), 1.69 (m, 1H), 1.54 (s, 3H), 1.34 (m, 1H), 1.19 (m, 1H), 0.93 (t, J=7.2 Hz, 3H).
Compound 29: A mixture of compound 21 (70 mg, 0.14 mmol) and hydrido(dimethylphosphinous acid-kP)[hydrogen bis(dimethylphosphinito-kP)]platinum(II) (6.1 mg, 14 mmol) in EtOH (1 mL) and water (1 mL) were heated at 90° C. for 4 h open to the air. After cooled to room temperature, the mixture was concentrated. The residue was extracted with EtOAc (2×15 mL). The organic extract was dried with Na₂SO₄, filtered, and concentrated. The residue was purified by column chromatography (silica gel, eluting with 0 to 100% EtOAc in hexanes) to give impure product, which was purified again by column chromatography (silica gel, eluting with 0 to 50% acetone in hexanes) to give compound 29 (33 mg, 45% yield) as a white solid. m/ [symbol] =511 (M+1); ¹H NMR (400 MHz, CDCl₃) δ 9.47 (s, 1H), 8.58 (d, J=5.1 Hz, 1H), 8.50 (d, J=5.1 Hz, 1H), 8.24 (s, 1H), 8.13 (dd, J=5.1, 1.6 Hz, 1H), 7.53-7.42 (m, 2H), 7.32 (t, J=7.5 Hz, 1H), 7.20 (t, J=9.1 Hz, 1H), 5.68 (d, J=4.5 Hz, 1H), 2.89-2.70 (m, 2H), 2.62 (ddd, J=12.9, 5.2, 3.4 Hz, 1H), 2.42 (td, J=12.8, 2.5 Hz, 1H), 2.19 (m, 1H), 2.14-1.99 (m, 2H), 1.91-1.63 (m, 2H), 1.50 (s, 3H), 1.43-1.13 (m, 2H), 1.10 (m, 2H), 1.02 (m, 2H), 0.93 (t, J=7.3 Hz, 3H).
Compound 30: A solution of compound I-11 (225 mg, 0.59 mmol) and 2-iodoxybenzoic acid (657 mg, 2.35 mmol) in DMSO (5 mL) was heated at 80-85° C. under nitrogen for 20 h. The reaction mixture was cooled to room temperature and diluted with t-butyl methyl ether (50 mL). The mixture was stirred for 10 min and filtered through a pad of Celite®. The filter cake was washed with t-butyl methyl ether (2×25 mL). The filtrate was washed with water (3×50 mL). The organic extract was dried with Na₂SO₄, filtered, and concentrated. The residue was purified by column chromatography (silica gel, eluting with 0 to 70% EtOAc in hexanes) to give a mixture of compound I-11 and compound 30 (150 mg).

The mixture of compound I-11 and compound 30 (72 mg, 0.19 mmol) in ethylene glycol (1 mL) was treated with p-toluenesulfonic acid monohydrate (35.7 mg, 0.19 mmol) at at room temperature. The mixture was stirred at room temperature for 30 min, compound I-11 was converted to compound I-12. The mixture was diluted with CH₂Cl₂ (20 mL), poured into sat. aq. NaHCO₃ (20 mL), and stirred for 5 min. The organic layer was separated. The aqueous layer was extracted with CH₂Cl₂ (20 mL). The combined organic extracts were dried with Na₂SO₄, filtered, and concentrated. The residue was purified by column chromatography (silica gel, eluting with 0 to 50% acetone in hexnaes) to give compound 30 (32 mg, 30% yield from I-11) as a white solid. m/z=382 (M+1); ¹H NMR (400 MHz, CDCl₃) δ 8.76 (m, 2H), 8.38 (m, 2H), 8.28 (d, J=10.1 Hz, 1H), 7.63-7.58 (m, 2H), 7.55-7.49 (m, 3H), 6.12 (d, J=10.1 Hz, 1H), 3.06-2.89 (m, 2H), 2.53 (dq, J=13.5, 6.8 Hz, 1H), 2.21 (td, J=12.7, 2.7 Hz, 1H), 2.11 (m, 1H), 1.75 (m, 1H), 1.47 (s, 3H), 1.27 (d, J=6.7 Hz, 3H).
Compound 32: A mixture of compound I-13 (35 mg, 0.086 mmol) and hydrido(dimethylphosphinous acid-kP)[hydrogen bis(dimethylphosphinito-kP)]platinum(II) (3.5 mg, 8.6 mmol) in EtOH (1 mL) and water (1 mL) were heated at 90° C. for 4 h open to the air. After cooled to room temperature, the mixture was concentrated. The residue was extracted with EtOAc. The organic extract was dried with Na₂SO₄, filtered, and concentrated. The residue was purified by column chromatography (silica gel, eluting 0 to 100% EtOAc in hexanes) to give impure product, which was purified again by column chromatography (silica gel, eluting 0 to 50% acetone in hexanes) to give compound 32 (mixture of tautomers, 8 mg, 22% yield) as a white solid. m/ [symbol] =427 (M+1); ¹H NMR (400 MHz, CDCl₃) δ major tautomer: 8.73 (m, 2H), 8.32 (m, 2H), 7.65-7.59 (m, 2H), 7.54-7.48 (m, 3H), 4.78 (bs, 2H), 3.06 (d, J=14.5 Hz, 1H), 2.99-2.80 (m, 2H), 2.39 (dd, J=14.6, 2.6 Hz, 1H), 2.29 (m, 1H), 2.05 (m, 1H), 1.69 (ddd, J=12.9, 11.0, 2.3 Hz, 1H), 1.50 (m, 1H), 1.32 (d, J=6.8 Hz, 3H), 1.28 (s, 3H).

All of the compounds, compositions, and methods disclosed and claimed herein can be made and executed without undue experimentation in light of the present disclosure. While the disclosure may have focused on several embodiments or may have been described in terms of preferred embodiments, it will be apparent to those of skill in the art that variations and modifications may be applied to the compounds, compositions, and methods without departing from the spirit, scope, and concept of the invention. All variations and modifications apparent to those skilled in the art are deemed to be within the spirit, scope, and concept of the invention as defined by the appended claims.

### REFERENCES

The following references to the extent that they provide exemplary procedural or other details supplementary to those set forth herein, are specifically incorporated herein by reference.
PCT/US2013/045975 (WO 2013/188818)
PCT/US2017/000094 (WO 2018/111315)
PCT/US2019/037543
Anderson, Practical Process Research & Development—A Guide for Organic Chemists, 2^{nd} ed., Academic Press, New York, 2012.
Bronner, et al., Expert Opin. Ther. Pat., 1:101-112, 2017.
Coltart and Danishefsky, Org. Lett., 5:1289, 2003.
Fujiwara, et al., J. Immunol., 193(5):2565-73, 2014.
Handbook of Pharmaceutical Salts: Properties, and Use, Stahl and Wermuth Eds., Verlag Helvetica Chimica Acta, 2002.
Lu et al., J. Clin. Invest., 121(10):4015-29, 2011.
Miosse and Kolls, Nature Reviews, 11(10):763-776, 2012.
Reagan-Shaw et al., FASEB J., 22(3):659-661, 2008.
Smith, March's Advanced Organic Chemistry: Reactions, Mechanisms, and Structure, 7^{th} Ed., Wiley, 2013.
Waite and Skokos, International Journal of Inflammation, 2012:1-10, 2011.

1. A method of treating a disease or disorder in a patient in need thereof, comprising administering to the patient a therapeutically effective amount of a cysteine-dependent inverse agonist of the nuclear receptor RORγ/RORγt.

2.-165. (canceled)

166. A compound of the formula: 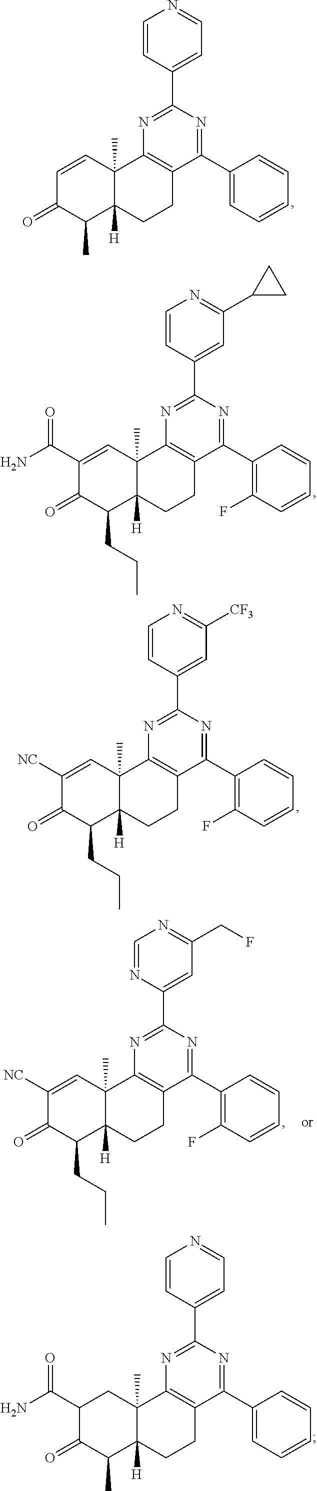
or a pharmaceutically acceptable salt thereof.

167. A pharmaceutical composition comprising:
(A) a compound according to claim 166; and
(B) an excipient.

168. The pharmaceutical composition of claim 167, wherein the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion.

169. The pharmaceutical composition of claim 168, wherein the pharmaceutical composition is formulated for oral administration.

170. The pharmaceutical composition of claim 168, wherein the pharmaceutical composition is formulated for administration via injection.

171. The pharmaceutical composition of claim 170, wherein the pharmaceutical composition is formulated for intraarterial administration, intramuscular administration, intraperitoneal administration, or intravenous administration.

172. The pharmaceutical composition of claim 168, wherein the pharmaceutical composition is formulated for administration topically.

173. The pharmaceutical composition of claim 172, wherein the pharmaceutical composition is formulated for topical administration to the skin or to the eye.

174. The pharmaceutical composition of claim 167, wherein the pharmaceutical composition is formulated as a unit dose.

175. The compound of claim 166 further defined as: 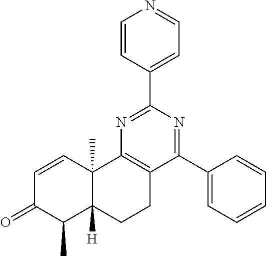 [SMILES: C[C@H]1C(=O)C=C[C@@]2(C)c3nc(-c4ccncc4)nc(-c4ccccc4)c3CC[C@H]12]
or a pharmaceutically acceptable salt thereof.

176. The compound of claim 166 further defined as: 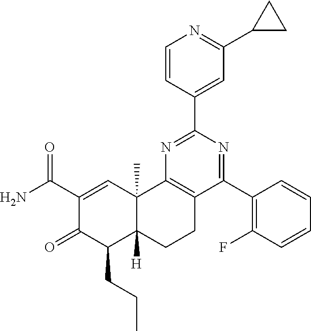 [SMILES: CCC[C@H]1C(=O)C(C(N)=O)=C[C@@]2(C)c3nc(-c4ccnc(C5CC5)c4)nc(-c4ccccc4F)c3CC[C@H]12]
or a pharmaceutically acceptable salt thereof.

177. The compound of claim 166 further defined as: 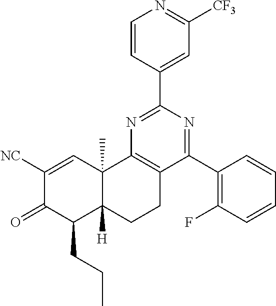 [SMILES: CCC[C@H]1C(=O)C(C#N)=C[C@@]2(C)c3nc(-c4ccnc(C(F)(F)F)c4)nc(-c4ccccc4F)c3CC[C@H]12]
or a pharmaceutically acceptable salt thereof.

178. The compound of claim 166 further defined as: 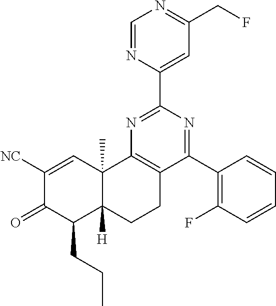 [SMILES: CCC[C@H]1C(=O)C(C#N)=C[C@@]2(C)c3nc(-c4cc(CF)ncn4)nc(-c4ccccc4F)c3CC[C@H]12]
or a pharmaceutically acceptable salt thereof.

179. The compound of claim 166 further defined as: 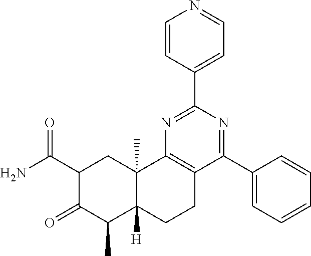 [SMILES: C[C@H]1C(=O)C(C(N)=O)C[C@@]2(C)c3nc(-c4ccncc4)nc(-c4ccccc4)c3CC[C@H]12]
or a pharmaceutically acceptable salt thereof.

180. The pharmaceutical composition of claim 167, wherein the compound is further defined as: 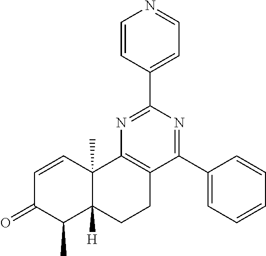 [SMILES: C[C@H]1C(=O)C=C[C@@]2(C)c3nc(-c4ccncc4)nc(-c4ccccc4)c3CC[C@H]12]
or a pharmaceutically acceptable salt thereof.

181. The pharmaceutical composition of claim 167, wherein the compound is further defined as: 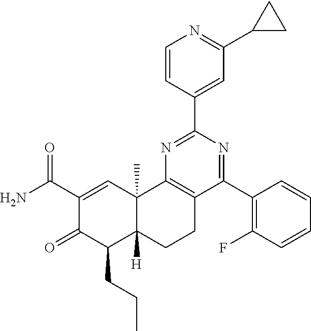 [SMILES: CCC[C@H]1C(=O)C(C(N)=O)=C[C@@]2(C)c3nc(-c4ccnc(C5CC5)c4)nc(-c4ccccc4F)c3CC[C@H]12]
or a pharmaceutically acceptable salt thereof.

182. The pharmaceutical composition of claim 167, wherein the compound is further defined as: 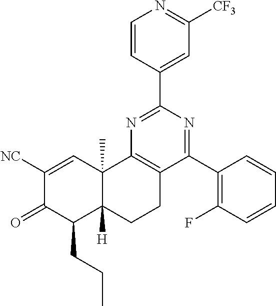 [SMILES: CCC[C@H]1C(=O)C(C#N)=C[C@@]2(C)c3nc(-c4ccnc(C(F)(F)F)c4)nc(-c4ccccc4F)c3CC[C@H]12]
or a pharmaceutically acceptable salt thereof.

183. The pharmaceutical composition of claim 167, wherein the compound is further defined as: 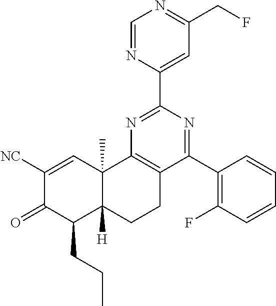 [SMILES: CCC[C@H]1C(=O)C(C#N)=C[C@@]2(C)c3nc(-c4cc(CF)ncn4)nc(-c4ccccc4F)c3CC[C@H]12]
or a pharmaceutically acceptable salt thereof.

184. The pharmaceutical composition of claim 167, wherein the compound is further defined as: 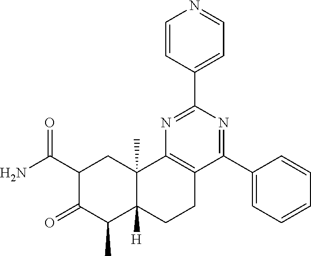 [SMILES: C[C@H]1C(=O)C(C(N)=O)C[C@@]2(C)c3nc(-c4ccncc4)nc(-c4ccccc4)c3CC[C@H]12]
or a pharmaceutically acceptable salt thereof.


SEQUENCE LISTING (2 sequences)

SEQ ID NO: 1 - protein, 518 aa, Homo sapiens
MDRAPQRQHRASRELLAAKKTHTSQIEVIPCKICGDKSSGIHYGVITCEGCKGFFRRSQRCNAAYSCTRQQNCPIDRTSRNRCQHCRLQKCLALGMSRDAVKFGRMSKKQRDSLHAEVQKQLQQRQQQQQEPVVKTPPAGAQGADTLTYTLGLPDGQLPLGSSPDLPEASACPPGLLKASGSGPSYSNNLAKAGLNGASCHLEYSPERGKAEGRESFYSTGSQLTPDRCGLRFEEHRHPGLGELGQGPDSYGSPSFRSTPEAPYASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFSTETESPVGLSK

SEQ ID NO: 2 - protein, 497 aa, Homo sapiens
MRTQIEVIPCKICGDKSSGIHYGVITCEGCKGFFRRSQRCNAAYSCTRQQNCPIDRTSRNRCQHCRLQKCLALGMSRDAVKFGRMSKKQRDSLHAEVQKQLQQRQQQQQEPVVKTPPAGAQGADTLTYTLGLPDGQLPLGSSPDLPEASACPPGLLKASGSGPSYSNNLAKAGLNGASCHLEYSPERGKAEGRESFYSTGSQLTPDRCGLRFEEHRHPGLGELGQGPDSYGSPSFRSTPEAPYASLTEIEHLVQSVCKSYRETCQLRLEDLLRQRSNIFSREEVTGYQRKSMWEMWERCAHHLTEAIQYVVEFAKRLSGFMELCQNDQIVLLKAGAMEVVLVRMCRAYNADNRTVFFEGKYGGMELFRALGCSELISSIFDFSHSLSALHFSEDEIALYTALVLINAHRPGLQEKRKVEQLQYNLELAFHHHLCKTHRQSILAKLPPKGKLRSLCSQHVERLQIFQHLHPIVVQAAFPPLYKELFSTETESPVGLSK